(19) United States
(12) Patent Application Publication
(10) Pub. No.: US 2024/0190982 A1
(43) Pub. Date: Jun. 13, 2024

(54) COMBINATION THERAPIES COMPRISING AN ANTI-GITR ANTIBODY FOR TREATING CANCERS

(71) Applicant: Incyte Corporation, Wilmington, DE (US)

(72) Inventors: Sonia Ioannidis, Morges (CH); Nawel Bourayou, Morges (CH)

(21) Appl. No.: 18/386,380

(22) Filed: Nov. 2, 2023

Related U.S. Application Data
(60) Provisional application No. 63/422,149, filed on Nov. 3, 2022.
(60) Provisional application No. 63/461,634, filed on Apr. 25, 2023.

(51) Int. Cl. C07K 16/28; A61P 35/00

(52) U.S. Cl. CPC C07K 16/2878; A61P 35/00; C07K 16/2818; A61K 2039/505

(57) ABSTRACT

Methods of treating a cancer by administering to a subject an anti-GITR antibody in combination with at least one of a PD-1 inhibitor (e.g., an anti-PD-1 inhibitor), a PD-L1 inhibitor (e.g., an anti-PD-L1 antibody), or a CTLA-4 inhibitor (e.g., an anti-CTLA-4 antibody) are disclosed herein.

### CLAIM OF PRIORITY

This application claims the benefit of U.S. Provisional Patent Application No. 63/422,149, filed on Nov. 3, 2022, and U.S. Provisional Patent Application No. 63/461,634, filed on Apr. 25, 2023, each of which is incorporated by reference herein in its entirety.

### SEQUENCE LISTING

This application contains a Sequence Listing that has been submitted electronically as an XML file named 20443-0787001_SL.xml. The XML file, created on Nov. 1, 2023, is 104,927 bytes in size. The material in the XML file is hereby incorporated by reference in its entirety.

### TECHNICAL FIELD

The present invention is directed to pharmaceutical methods of treating a cancer by administering to a subject a combination of an anti-GITR antibody and at least one of a PD-1 inhibitor, a PD-L1 inhibitor, or a CTLA-4 inhibitor.

### BACKGROUND

Cancer is among the leading causes of death worldwide. Many patients are diagnosed with advanced disease, have no response to treatment, or have a response to treatment that is followed by disease progression. Thus, there is a need for therapies targeting cancer.

### SUMMARY

The present disclosure is based, at least in part, on the development of cancer treatments using a combination therapy comprising an anti-GITR antibody and at least one of a PD-1 inhibitor (e.g., an anti-PD-1 antibody), a PD-L1 inhibitor (e.g., an anti-PD-L1 antibody), or a CTLA-4 inhibitor.

Accordingly, aspects of the present disclosure provide a method for treating a cancer in a human subject in need thereof, the method comprising administering to the human subject an effective amount of an anti-GITR antibody and an anti-PD-1 antibody, wherein the anti-GITR antibody comprises a heavy chain variable region (VH) comprising a VH CDR1, a VH CDR2, and a VH CDR3, and a light chain variable region (VL) comprising a VL CDR1, a VL CDR2, and a VL CDR3, wherein the VH CDR1 comprises the amino acid sequence of DYAMY (SEQ ID NO:1), the VH CDR2 comprises the amino acid sequence of VIRTYSGDVTYNQKFKD (SEQ ID NO:2), the VH CDR3 comprises the amino acid sequence of SGTVRGFAY (SEQ ID NO:3), the VL CDR1 comprises the amino acid sequence of KSSQSLLNSGNQKNYLT (SEQ ID NO:4), the VL CDR2 comprises the amino acid sequence of WASTRES (SEQ ID NO:5), and the VL CDR3 comprises the amino acid sequence of QNDYSYPYT (SEQ ID NO:6).

In some embodiments, the method does not comprise administration of a multivalent autophagosome cancer vaccine to the human subject. In some embodiments, the method does not comprise administration of a cancer vaccine to the human subject.

In some embodiments, the human subject has an advanced solid tumor, advanced renal cell carcinoma, anal cancer, bladder cancer, brain cancer, breast cancer, cervical cancer, colorectal cancer, endometrial cancer, esophageal cancer, gastric cancer, gastrointestinal cancer, gastroesophageal cancer, glioblastoma, head and neck squamous cell carcinoma (HNSCC), hepatocellular carcinoma (HCC), glioma, kidney cancer, liver cancer, lung cancer, lymphoma, melanoma, Merkel cell carcinoma, mesothelioma, multiple myeloma, myeloma, neuroblastoma, non-small cell lung cancer (NSCLC), ovarian cancer, pancreatic cancer, prostate cancer, rectal cancer, renal cell carcinoma (RCC), sarcoma, skin cancer, small cell lung cancer (SCLC), soft-tissue sarcoma, thoracic cancer, thyroid cancer, triple-negative breast cancer (TNBC), urothelial cancer, uterine cancer, or a combination thereof.

In some embodiments, the human subject has an advanced solid tumor, breast cancer, cervical cancer, colorectal cancer, endometrial cancer, esophageal cancer, gastric cancer, gastrointestinal cancer, glioblastoma, head and neck squamous cell carcinoma (HNSCC), hepatocellular carcinoma (HCC), liver cancer, lung cancer, lymphoma, melanoma, Merkel cell carcinoma, mesothelioma, non-small cell lung cancer (NSCLC), ovarian cancer, pancreatic cancer, prostate cancer, renal cell carcinoma (RCC), small cell lung cancer (SCLC), soft-tissue sarcoma, thoracic cancer, triple-negative breast cancer (TNBC), urothelial cancer, or a combination thereof.

In some embodiments, the human subject has an unresectable cancer, a recurrent cancer, or a metastatic cancer.

In some embodiments, the human subject has head and neck squamous cell carcinoma (HNSCC). In some embodiments, the human subject has recurrent or metastatic HNSCC. In some embodiments, HNSCC tumor cells in a biological sample obtained from the human subject express GITR. In some embodiments, the method comprises determining that HNSCC tumor cells in a biological sample obtained from the human subject express GITR. In some embodiments, at least 10% of HNSCC tumor cells in the biological sample obtained from the human subject express GITR.

In some embodiments, the human subject has undergone a prior anti-cancer therapy.

In some embodiments, the prior anti-cancer therapy comprises, an immune checkpoint inhibitor, a chemotherapy, or a combination thereof. In some embodiments, the immune checkpoint inhibitor is an anti-PD-(L)1 inhibitor.

In some embodiments, the human subject has progressed on or after prior systemic treatment. In some embodiments, the human subject has progressed on or after prior systemic anti-PD-(L)1 therapy.

In some embodiments, the anti-GITR antibody is administered to the human subject intravenously at a dose between 0.1 to 1000 mg. In some embodiments, the anti-GITR antibody is administered to the human subject intravenously at a dose of 300 mg. In some embodiments, the anti-GITR antibody is administered to the human subject intravenously at a dose of 600 mg.

In some embodiments, the anti-GITR antibody is administered to the human subject intravenously at a dose between 0.3 mg/kg to 10 mg/kg. In some embodiments, the anti-GITR antibody is administered to the human subject intravenously at a dose of 0.3 mg/kg, 1.0 mg/kg, 3.0 mg/kg, 5.0 mg/kg, or 10 mg/kg.

In some embodiments, the anti-PD-1 antibody is administered to the human subject intravenously at a dose between 100 to 1000 mg. In some embodiments, the anti-PD-1 antibody is administered to the human subject intravenously at a dose of 200 mg. In some embodiments, the anti-PD-1 antibody is administered to the human subject intravenously at a dose of 240 mg. In some embodiments, the anti-PD-1 antibody is administered to the human subject intravenously at a dose of 500 mg.

In some embodiments, the anti-PD-1 antibody is administered to the human subject intravenously at a dose of between 0.5 mg/kg to 5 mg/kg. In some embodiments, the anti-PD-1 antibody is administered to the human subject intravenously at a dose of 3 mg/kg.

In some embodiments, the anti-GITR antibody and the anti-PD-1 antibody are administered simultaneously or sequentially.

In some embodiments, the anti-GITR antibody is administered to the human subject once every two weeks. In some embodiments, the anti-GITR antibody is administered to the human subject once every three weeks. In some embodiments, the anti-GITR antibody is administered to the human subject once every four weeks.

In some embodiments, the anti-PD-1 antibody is administered to the human subject once every two weeks. In some embodiments, the anti-PD-1 antibody is administered to the human subject once every three weeks. In some embodiments, the anti-PD-1 antibody is administered to the human subject once every four weeks.

In some embodiments, the anti-GITR antibody is administered to the human subject intravenously once every two weeks at a dose between 10 to 750 mg. In some embodiments, the anti-GITR antibody is administered to the human subject intravenously once every two weeks at a dose of 300 mg. In some embodiments, the anti-GITR antibody is administered to the human subject intravenously once every two weeks at a dose of 600 mg.

In some embodiments, the anti-GITR antibody is administered to the human subject intravenously once every two weeks at a dose between 0.3 mg/kg to 10 mg/kg. In some embodiments, the anti-GITR antibody is administered to the human subject intravenously once every three weeks at a dose between 0.3 mg/kg to 10 mg/kg.

In some embodiments, the anti-PD-1 antibody is administered to the human subject intravenously once every two weeks at a dose of 240 mg. In some embodiments, the anti-PD-1 antibody is administered to the human subject intravenously once every two weeks at a dose of 3 mg/kg. In some embodiments, the anti-PD-1 antibody is administered to the human subject intravenously once every three weeks at a dose between 100 to 300 mg. In some embodiments, the anti-PD-1 antibody is administered to the human subject intravenously once every three weeks at a dose of 200 mg. In some embodiments, the anti-PD-1 antibody is administered to the human subject intravenously once every four weeks at a dose of 500 mg.

In some embodiments, the anti-GITR antibody is administered to the human subject intravenously once every two weeks at a dose of 300 mg, and wherein the anti-PD-1 antibody is administered to the human subject intravenously once every four weeks at a dose of 500 mg. In some embodiments, the human subject has HNSCC or glioblastoma.

In some embodiments, the anti-GITR antibody is administered to the human subject intravenously once every two weeks at a dose of 600 mg, and wherein the anti-PD-1 antibody is administered to the human subject intravenously once every four weeks at a dose of 500 mg. In some embodiments, the human subject has HNSCC.

In some embodiments, the anti-GITR antibody is administered between 12 to 28 days prior to administering the anti-PD-1 antibody.

In some embodiments, the anti-GITR antibody is administered to the human subject intravenously once every three weeks at a dose between 0.3 mg/kg to 10 mg/kg, and wherein the anti-PD-1 antibody is administered to the human subject intravenously once every three weeks at a dose of 200 mg. In some embodiments, the human subject has cervical cancer, endometrial cancer, gastric cancer, esophageal cancer, hepatocellular carcinoma (HCC), melanoma, Merkel cell carcinoma, mesothelioma, non-small cell lung cancer (NSCLC), ovarian cancer, head and neck squamous cell carcinoma (HNSCC), small cell lung cancer (SCLC), renal cell carcinoma (RCC), triple-negative breast cancer (TNBC), urothelial cancer, or a combination thereof.

In some embodiments, the anti-GITR antibody is administered to the human subject intravenously once every two weeks at a dose between 0.3 mg/kg to 10 mg/kg, and wherein the anti-PD-1 antibody is administered to the human subject intravenously once every two weeks at a dose of 240 mg or 3 mg/kg. In some embodiments, the human subject has cervical cancer, endometrial cancer, gastric cancer, esophageal cancer, hepatocellular carcinoma (HCC), melanoma, Merkel cell carcinoma, mesothelioma, non-small cell lung cancer (NSCLC), ovarian cancer, head and neck squamous cell carcinoma (HNSCC), small cell lung cancer (SCLC), renal cell carcinoma (RCC), triple-negative breast cancer (TNBC), urothelial cancer, or a combination thereof.

In some embodiments, the anti-GITR antibody is administered to the human subject intravenously once every two weeks at a dose of 300 mg, and wherein the anti-PD-1 antibody is administered to the human subject intravenously once every four weeks at a dose of 500 mg. In some embodiments, the human subject has glioblastoma.

In some embodiments, the anti-GITR antibody is administered to the human subject intravenously once every three weeks at a dose between 10 to 750 mg, and wherein the anti-PD-1 antibody is administered to the human subject intravenously once every three weeks at a dose between 100 to 300 mg. In some embodiments, the human subject has colorectal cancer, melanoma, ovarian cancer, breast cancer, cervical cancer, mesothelioma, pancreatic cancer, lymphoma, or a combination thereof.

In some embodiments, the anti-GITR antibody is administered to the human subject once every three weeks at a dose between 0.12 to 60 mg, and wherein the anti-PD-1 antibody is administered to the human subject once every three weeks at a dose of 200 mg. In some embodiments, the human subject has colorectal cancer, melanoma, renal cell carcinoma (RCC), ovarian cancer, breast cancer, cervical cancer, head and neck squamous cell carcinoma (HNSCC), hepatocellular carcinoma (HCC), non-small cell lung cancer (NSCLC), soft-tissue sarcoma, gastric cancer, gastrointestinal cancer, mesothelioma, pancreatic cancer, urothelial cancer, or a combination thereof.

In some embodiments, the anti-GITR antibody is administered to the human subject once every four weeks, and wherein the anti-PD-1 antibody is administered to the human subject once every four weeks. In some embodiments, the human subject has liver cancer, lung cancer, thoracic cancer, or a combination thereof.

In some embodiments, the VH of the anti-GITR antibody is at least 80% identical to the amino acid sequence of SEQ ID NO:7, and wherein the VL of the anti-GITR antibody is at least 80% identical to the amino acid sequence of SEQ ID NO:8.

In some embodiments, the VH of the anti-GITR antibody comprises the amino acid sequence of SEQ ID NO:7, and wherein the VL of the anti-GITR antibody comprises the amino acid sequence of SEQ ID NO:8.

In some embodiments, the anti-GITR antibody comprises a heavy chain that is at least 80% identical to the amino acid sequence of SEQ ID NO:9 and a light chain that is at least 80% identical to the amino acid sequence of SEQ ID NO:10.

In some embodiments, the anti-GITR antibody comprises a heavy chain comprising the amino acid sequence of SEQ ID NO:9 and a light chain comprising the amino acid sequence of SEQ ID NO:10.

In some embodiments, the anti-PD-1 antibody comprises a VH comprising a VH CDR1, a VH CDR2, and a VH CDR3, and a VL comprising a VL CDR1, a VL CDR2, and a VL CDR3, wherein the VH CDR1 comprises the amino acid sequence SYWMN (SEQ ID NO:11), the VH CDR2 comprises the amino acid sequence VIHPSDSETWLDQKFKD (SEQ ID NO:12), the VH CDR3 comprises the amino acid sequence EHYGTSPFAY (SEQ ID NO:13), the VL CDR1 comprises the amino acid sequence RASESVDNYGMSFMNW (SEQ ID NO:14), the VL CDR2 comprises the amino acid sequence AASNQGS (SEQ ID NO:15), and the VL CDR3 comprises the amino acid sequence QQSKEVPYT (SEQ ID NO:16).

In some embodiments, the VH of the anti-PD-1 antibody is at least 80% identical to the amino acid sequence of SEQ ID NO:17, and wherein the VL of the anti-PD-1 antibody is at least 80% identical to the amino acid sequence of SEQ ID NO:18.

In some embodiments, the VH of the anti-PD-1 antibody comprises the amino acid sequence of SEQ ID NO:17, and wherein the VL of the anti-PD-1 antibody comprises the amino acid sequence of SEQ ID NO:18.

In some embodiments, the anti-PD-1 antibody comprises a heavy chain that is at least 80% identical to the amino acid sequence of SEQ ID NO:19 and a light chain that is at least 80% identical to the amino acid sequence of SEQ ID NO:20.

In some embodiments, the anti-PD-1 antibody comprises a heavy chain comprising the amino acid sequence of SEQ ID NO:19 and a light chain comprising the amino acid sequence of SEQ ID NO:20.

In some embodiments, the anti-PD-1 antibody comprises a VH comprising a VH CDR1, a VH CDR2, and a VH CDR3, and a VL comprising a VL CDR1, a VL CDR2, and a VL CDR3, wherein the VH CDR1 comprises the amino acid sequence NYYMY (SEQ ID NO:21), the VH CDR2 comprises the amino acid sequence GINPSNGGTNFNEKFKN (SEQ ID NO:22), the VH CDR3 comprises the amino acid sequence RDYRFDMGFDY (SEQ ID NO:23), the VL CDR1 comprises the amino acid sequence RASKGVSTSGYSYLH (SEQ ID NO:24), the VL CDR2 comprises the amino acid sequence LASYLES (SEQ ID NO:25), and the VL CDR3 comprises the amino acid sequence QHSRDLPLT (SEQ ID NO:26).

In some embodiments, the VH of the anti-PD-1 antibody is at least 80% identical to the amino acid sequence of SEQ ID NO:27, and wherein the VL of the anti-PD-1 antibody is at least 80% identical to the amino acid sequence of SEQ ID NO:28.

In some embodiments, the VH of the anti-PD-1 antibody comprises the amino acid sequence of SEQ ID NO:27, and wherein the VL of the anti-PD-1 antibody comprises the amino acid sequence of SEQ ID NO:28.

In some embodiments, the anti-PD-1 antibody comprises a heavy chain that is at least 80% identical to the amino acid sequence of SEQ ID NO:29 and a light chain that is at least 80% identical to the amino acid sequence of SEQ ID NO:30.

In some embodiments, the anti-PD-1 antibody comprises a heavy chain comprising the amino acid sequence of SEQ ID NO:29 and a light chain comprising the amino acid sequence of SEQ ID NO:30.

In some embodiments, the anti-PD-1 antibody comprises a VH comprising a VH CDR1, a VH CDR2, and a VH CDR3, and a VL comprising a VL CDR1, a VL CDR2, and a VL CDR3, wherein the VH CDR1 comprises the amino acid sequence NSGMH (SEQ ID NO:31), the VH CDR2 comprises the amino acid sequence VIWYDGSKRYYADSVKG (SEQ ID NO:32), the VH CDR3 comprises the amino acid sequence NDDY (SEQ ID NO:33), the VL CDR1 comprises the amino acid sequence RASQSVSSYLA (SEQ ID NO:34), the VL CDR2 comprises the amino acid sequence DASNRAT (SEQ ID NO:35), and the VL CDR3 comprises the amino acid sequence QQSSNWPRT (SEQ ID NO:36).

In some embodiments, the VH of the anti-PD-1 antibody is at least 80% identical to the amino acid sequence of SEQ ID NO:37, and wherein the VL of the anti-PD-1 antibody is at least 80% identical to the amino acid sequence of SEQ ID NO:38.

In some embodiments, the VH of the anti-PD-1 antibody comprises the amino acid sequence of SEQ ID NO:37, and wherein the VL of the anti-PD-1 antibody comprises the amino acid sequence of SEQ ID NO:38.

In some embodiments, the anti-PD-1 antibody comprises a heavy chain that is at least 80% identical to the amino acid sequence of SEQ ID NO:39 and a light chain that is at least 80% identical to the amino acid sequence of SEQ ID NO:40.

In some embodiments, the anti-PD-1 antibody comprises a heavy chain comprising the amino acid sequence of SEQ ID NO:39 and a light chain comprising the amino acid sequence of SEQ ID NO:40.

In some embodiments, the anti-PD-1 antibody comprises a VH comprising a VH CDR1, a VH CDR2, and a VH CDR3, and a VL comprising a VL CDR1, a VL CDR2, and a VL CDR3, wherein the VH CDR1 comprises the amino acid sequence NSGMH (SEQ ID NO:41), the VH CDR2 comprises the amino acid sequence VIWYDGSKRYYADSVKG (SEQ ID NO:42), the VH CDR3 comprises the amino acid sequence NDDY (SEQ ID NO:43), the VL CDR1 comprises the amino acid sequence RASQSVSSYLA (SEQ ID NO:44), the VL CDR2 comprises the amino acid sequence DASNRAT (SEQ ID NO:45), and the VL CDR3 comprises the amino acid sequence QQSSNWPRT (SEQ ID NO:46).

In some embodiments, the VH of the anti-PD-1 antibody is at least 80% identical to the amino acid sequence of SEQ ID NO:47, and wherein the VL of the anti-PD-1 antibody is at least 80% identical to the amino acid sequence of SEQ ID NO:48.

In some embodiments, the VH of the anti-PD-1 antibody comprises the amino acid sequence of SEQ ID NO:47, and wherein the VL of the anti-PD-1 antibody comprises the amino acid sequence of SEQ ID NO:48.

In some embodiments, the anti-PD-1 antibody comprises a heavy chain that is at least 80% identical to the amino acid sequence of SEQ ID NO:49 and a light chain that is at least 80% identical to the amino acid sequence of SEQ ID NO:50.

In some embodiments, the anti-PD-1 antibody comprises a heavy chain comprising the amino acid sequence of SEQ ID NO:49 and a light chain comprising the amino acid sequence of SEQ ID NO:50.

In some embodiments, the human subject does not show one or more of the following features:
(a) prior treatment with an anticancer medication within the last 30 days;
(b) prior treatment with a tumor necrosis factor superfamily (TNFSF) agonist;
(c) significant worsening of clinical status;
(d) brain or central nervous system (CNS) metastases;
(e) active infection;
(f) chronic systemic steroid use;
(g) lung disease or pneumonitis; and
(h) vaccine use.

In some embodiments, the human subject has HNSCC, wherein the anti-GITR antibody comprises a heavy chain comprising the amino acid sequence of SEQ ID NO:9 and a light chain comprising the amino acid sequence of SEQ ID NO:10, and wherein the anti-PD-1 antibody comprises a heavy chain comprising the amino acid sequence of SEQ ID NO:19 and a light chain comprising the amino acid sequence of SEQ ID NO:20.

In some embodiments, the human subject has HNSCC, wherein the anti-GITR antibody is administered to the human subject intravenously once every two weeks at a dose of 300 mg, wherein the anti-PD-1 antibody is administered to the human subject intravenously once every four weeks at a dose of 500 mg, wherein the anti-GITR antibody comprises a heavy chain comprising the amino acid sequence of SEQ ID NO:9 and a light chain comprising the amino acid sequence of SEQ ID NO:10, and wherein the anti-PD-1 antibody comprises a heavy chain comprising the amino acid sequence of SEQ ID NO:19 and a light chain comprising the amino acid sequence of SEQ ID NO:20.

In some embodiments, the human subject has HNSCC, wherein the anti-GITR antibody is administered to the human subject intravenously once every two weeks at a dose of 600 mg, wherein the anti-PD-1 antibody is administered to the human subject intravenously once every four weeks at a dose of 500 mg, wherein the anti-GITR antibody comprises a heavy chain comprising the amino acid sequence of SEQ ID NO:9 and a light chain comprising the amino acid sequence of SEQ ID NO:10, and wherein the anti-PD-1 antibody comprises a heavy chain comprising the amino acid sequence of SEQ ID NO:19 and a light chain comprising the amino acid sequence of SEQ ID NO:20.

In some embodiments, the human subject has cervical cancer, endometrial cancer, gastric cancer, esophageal cancer, hepatocellular carcinoma (HCC), melanoma, Merkel cell carcinoma, mesothelioma, non-small cell lung cancer (NSCLC), ovarian cancer, head and neck squamous cell carcinoma (HNSCC), small cell lung cancer (SCLC), renal cell carcinoma (RCC), triple-negative breast cancer (TNBC), urothelial cancer, or a combination thereof, wherein the anti-GITR antibody comprises a heavy chain comprising the amino acid sequence of SEQ ID NO:9 and a light chain comprising the amino acid sequence of SEQ ID NO:10, and wherein the anti-PD-1 antibody comprises a heavy chain comprising the amino acid sequence of SEQ ID NO:39 and a light chain comprising the amino acid sequence of SEQ ID NO:40.

In some embodiments, the human subject has cervical cancer, endometrial cancer, gastric cancer, esophageal cancer, hepatocellular carcinoma (HCC), melanoma, Merkel cell carcinoma, mesothelioma, non-small cell lung cancer (NSCLC), ovarian cancer, head and neck squamous cell carcinoma (HNSCC), small cell lung cancer (SCLC), renal cell carcinoma (RCC), triple-negative breast cancer (TNBC), urothelial cancer, or a combination thereof, wherein the anti-GITR antibody is administered to the human subject intravenously once every two weeks at a dose between 0.3 mg/kg to 10 mg/kg, wherein the anti-PD-1 antibody is administered to the human subject intravenously once every two weeks at a dose of 240 mg, wherein the anti-GITR antibody comprises a heavy chain comprising the amino acid sequence of SEQ ID NO:9 and a light chain comprising the amino acid sequence of SEQ ID NO:10, and wherein the anti-PD-1 antibody comprises a heavy chain comprising the amino acid sequence of SEQ ID NO:39 and a light chain comprising the amino acid sequence of SEQ ID NO:40.

In some embodiments, the human subject has cervical cancer, endometrial cancer, gastric cancer, esophageal cancer, hepatocellular carcinoma (HCC), melanoma, Merkel cell carcinoma, mesothelioma, non-small cell lung cancer (NSCLC), ovarian cancer, head and neck squamous cell carcinoma (HNSCC), small cell lung cancer (SCLC), renal cell carcinoma (RCC), triple-negative breast cancer (TNBC), urothelial cancer, or a combination thereof, wherein the anti-GITR antibody is administered to the human subject intravenously once every two weeks at a dose between 0.3 mg/kg to 10 mg/kg, wherein the anti-PD-1 antibody is administered to the human subject intravenously once every two weeks at a dose of 3 mg/kg, wherein the anti-GITR antibody comprises a heavy chain comprising the amino acid sequence of SEQ ID NO:9 and a light chain comprising the amino acid sequence of SEQ ID NO:10, and wherein the anti-PD-1 antibody comprises a heavy chain comprising the amino acid sequence of SEQ ID NO:39 and a light chain comprising the amino acid sequence of SEQ ID NO:40.

In some embodiments, the human subject has cervical cancer, endometrial cancer, gastric cancer, esophageal cancer, hepatocellular carcinoma (HCC), melanoma, Merkel cell carcinoma, mesothelioma, non-small cell lung cancer (NSCLC), ovarian cancer, head and neck squamous cell carcinoma (HNSCC), small cell lung cancer (SCLC), renal cell carcinoma (RCC), triple-negative breast cancer (TNBC), urothelial cancer, or a combination thereof, wherein the anti-GITR antibody is administered to the human subject intravenously once every two weeks at a dose of 300 mg, wherein the anti-PD-1 antibody is administered to the human subject intravenously once every two weeks at a dose of 240 mg, wherein the anti-GITR antibody comprises a heavy chain comprising the amino acid sequence of SEQ ID NO:9 and a light chain comprising the amino acid sequence of SEQ ID NO:10, and wherein the anti-PD-1 antibody comprises a heavy chain comprising the amino acid sequence of SEQ ID NO:39 and a light chain comprising the amino acid sequence of SEQ ID NO:40.

In some embodiments, the human subject has cervical cancer, endometrial cancer, gastric cancer, esophageal cancer, hepatocellular carcinoma (HCC), melanoma, Merkel cell carcinoma, mesothelioma, non-small cell lung cancer (NSCLC), ovarian cancer, head and neck squamous cell carcinoma (HNSCC), small cell lung cancer (SCLC), renal cell carcinoma (RCC), triple-negative breast cancer (TNBC), urothelial cancer, or a combination thereof, wherein the anti-GITR antibody is administered to the human subject intravenously once every three weeks at a dose of 300 mg, wherein the anti-PD-1 antibody is administered to the human subject intravenously once every two weeks at a dose of 240 mg, wherein the anti-GITR antibody comprises a heavy chain comprising the amino acid sequence of SEQ ID NO:9 and a light chain comprising the amino acid sequence of SEQ ID NO:10, and wherein the anti-PD-1 antibody comprises a heavy chain comprising the amino acid sequence of SEQ ID NO:39 and a light chain comprising the amino acid sequence of SEQ ID NO:40.

In some embodiments, the human subject has cervical cancer, endometrial cancer, gastric cancer, esophageal cancer, hepatocellular carcinoma (HCC), melanoma, Merkel cell carcinoma, mesothelioma, non-small cell lung cancer (NSCLC), ovarian cancer, head and neck squamous cell carcinoma (HNSCC), small cell lung cancer (SCLC), renal cell carcinoma (RCC), triple-negative breast cancer (TNBC), urothelial cancer, or a combination thereof, wherein the anti-GITR antibody comprises a heavy chain comprising the amino acid sequence of SEQ ID NO:9 and a light chain comprising the amino acid sequence of SEQ ID NO:10, and wherein the anti-PD-1 antibody comprises a heavy chain comprising the amino acid sequence of SEQ ID NO:29 and a light chain comprising the amino acid sequence of SEQ ID NO:30.

In some embodiments, the human subject has cervical cancer, endometrial cancer, gastric cancer, esophageal cancer, hepatocellular carcinoma (HCC), melanoma, Merkel cell carcinoma, mesothelioma, non-small cell lung cancer (NSCLC), ovarian cancer, head and neck squamous cell carcinoma (HNSCC), small cell lung cancer (SCLC), renal cell carcinoma (RCC), triple-negative breast cancer (TNBC), urothelial cancer, or a combination thereof, wherein the anti-GITR antibody is administered to the human subject intravenously once every two weeks at a dose of 300 mg, wherein the anti-PD-1 antibody is administered to the human subject intravenously once every two weeks at a dose of 3 mg/kg, wherein the anti-GITR antibody comprises a heavy chain comprising the amino acid sequence of SEQ ID NO:9 and a light chain comprising the amino acid sequence of SEQ ID NO:10, and wherein the anti-PD-1 antibody comprises a heavy chain comprising the amino acid sequence of SEQ ID NO:39 and a light chain comprising the amino acid sequence of SEQ ID NO:40.

In some embodiments, the human subject has cervical cancer, endometrial cancer, gastric cancer, esophageal cancer, hepatocellular carcinoma (HCC), melanoma, Merkel cell carcinoma, mesothelioma, non-small cell lung cancer (NSCLC), ovarian cancer, head and neck squamous cell carcinoma (HNSCC), small cell lung cancer (SCLC), renal cell carcinoma (RCC), triple-negative breast cancer (TNBC), urothelial cancer, or a combination thereof, wherein the anti-GITR antibody is administered to the human subject intravenously once every three weeks at a dose of 300 mg, wherein the anti-PD-1 antibody is administered to the human subject intravenously once every two weeks at a dose of 3 mg/kg, wherein the anti-GITR antibody comprises a heavy chain comprising the amino acid sequence of SEQ ID NO:9 and a light chain comprising the amino acid sequence of SEQ ID NO:10, and wherein the anti-PD-1 antibody comprises a heavy chain comprising the amino acid sequence of SEQ ID NO:39 and a light chain comprising the amino acid sequence of SEQ ID NO:40.

In some embodiments, the human subject has cervical cancer, endometrial cancer, gastric cancer, esophageal cancer, hepatocellular carcinoma (HCC), melanoma, Merkel cell carcinoma, mesothelioma, non-small cell lung cancer (NSCLC), ovarian cancer, head and neck squamous cell carcinoma (HNSCC), small cell lung cancer (SCLC), renal cell carcinoma (RCC), triple-negative breast cancer (TNBC), urothelial cancer, or a combination thereof, wherein the anti-GITR antibody is administered to the human subject intravenously once every three weeks at a dose between 0.3 mg/kg to 10 mg/kg, wherein the anti-PD-1 antibody is administered to the human subject intravenously once every three weeks at a dose of 200 mg, wherein the anti-GITR antibody comprises a heavy chain comprising the amino acid sequence of SEQ ID NO:9 and a light chain comprising the amino acid sequence of SEQ ID NO:10, and wherein the anti-PD-1 antibody comprises a heavy chain comprising the amino acid sequence of SEQ ID NO:29 and a light chain comprising the amino acid sequence of SEQ ID NO:30.

In some embodiments, the human subject is administered an additional anti-cancer therapy. In some embodiments, the additional anti-cancer therapy is an immune checkpoint inhibitor or an indoleamine 2,3-dioxygenase-1 (IDO1) inhibitor. In some embodiments, the immune checkpoint inhibitor is an anti-CTLA-4 antibody. In some embodiments, the anti-CTLA-4 antibody is ipilimumab. In some embodiments, the IDO1 inhibitor is epacadostat.

In some embodiments, the human subject is at least 18 years of age.

Aspects of the present disclosure provide a method for treating a cancer in a human subject in need thereof, the method comprising administering to the human subject an effective amount of an anti-GITR antibody and an anti-PD-L1 antibody, wherein the anti-GITR antibody comprises a heavy chain variable region (VH) comprising a VH CDR1, a VH CDR2, and a VH CDR3, and a light chain variable region (VL) comprising a VL CDR1, a VL CDR2, and a VL CDR3, wherein the VH CDR1 comprises the amino acid sequence of DYAMY (SEQ ID NO:1), the VH CDR2 comprises the amino acid sequence of VIRTYSGDVTYNQKFKD (SEQ ID NO:2), the VH CDR3 comprises the amino acid sequence of SGTVRGFAY (SEQ ID NO:3), the VL CDR1 comprises the amino acid sequence of KSSQSLLNSGNQKNYLT (SEQ ID NO:4), the VL CDR2 comprises the amino acid sequence of WASTRES (SEQ ID NO:5), and the VL CDR3 comprises the amino acid sequence of QNDYSYPYT (SEQ ID NO:6).

In some embodiments, the human subject has an advanced solid tumor, advanced renal cell carcinoma, anal cancer, bladder cancer, brain cancer, breast cancer, cervical cancer, colorectal cancer, endometrial cancer, esophageal cancer, gastric cancer, gastrointestinal cancer, gastroesophageal cancer, glioblastoma, head and neck squamous cell carcinoma (HNSCC), hepatocellular carcinoma (HCC), glioma, kidney cancer, liver cancer, lung cancer, lymphoma, melanoma, Merkel cell carcinoma, mesothelioma, multiple myeloma, myeloma, neuroblastoma, non-small cell lung cancer (NSCLC), ovarian cancer, pancreatic cancer, prostate cancer, rectal cancer, renal cell carcinoma (RCC), sarcoma, skin cancer, small cell lung cancer (SCLC), soft-tissue sarcoma, thoracic cancer, thyroid cancer, triple-negative breast cancer (TNBC), urothelial cancer, uterine cancer, or a combination thereof.

In some embodiments, the human subject has an advanced solid tumor, breast cancer, cervical cancer, colorectal cancer, endometrial cancer, esophageal cancer, gastric cancer, gastrointestinal cancer, glioblastoma, head and neck squamous cell carcinoma (HNSCC), hepatocellular carcinoma (HCC), liver cancer, lung cancer, lymphoma, melanoma, Merkel cell carcinoma, mesothelioma, non-small cell lung cancer (NSCLC), ovarian cancer, pancreatic cancer, prostate cancer, renal cell carcinoma (RCC), small cell lung cancer (SCLC), soft-tissue sarcoma, thoracic cancer, triple-negative breast cancer (TNBC), urothelial cancer, or a combination thereof.

In some embodiments, the VH of the anti-GITR antibody is at least 80% identical to the amino acid sequence of SEQ ID NO:7, and wherein the VL of the anti-GITR antibody is at least 80% identical to the amino acid sequence of SEQ ID NO:8.

In some embodiments, the VH of the anti-GITR antibody comprises the amino acid sequence of SEQ ID NO:7, and wherein the VL of the anti-GITR antibody comprises the amino acid sequence of SEQ ID NO:8.

In some embodiments, the anti-GITR antibody comprises a heavy chain that is at least 80% identical to the amino acid sequence of SEQ ID NO:9 and a light chain that is at least 80% identical to the amino acid sequence of SEQ ID NO:10.

In some embodiments, the anti-GITR antibody comprises a heavy chain comprising the amino acid sequence of SEQ ID NO:9 and a light chain comprising the amino acid sequence of SEQ ID NO:10.

In some embodiments, the anti-PD-L1 antibody comprises atezolizumab, avelumab, or durvalumab.

In some embodiments, the anti-GITR antibody is administered to the human subject intravenously at a dose between 0.1 to 1000 mg. In some embodiments, the anti-GITR antibody is administered to the human subject intravenously at a dose of 300 mg. In some embodiments, the anti-GITR antibody is administered to the human subject intravenously at a dose of 600 mg.

In some embodiments, the anti-GITR antibody is administered to the human subject intravenously at a dose between 0.3 mg/kg to 10 mg/kg. In some embodiments, the anti-GITR antibody is administered to the human subject intravenously at a dose of 0.3 mg/kg, 1.0 mg/kg, 3.0 mg/kg, 5.0 mg/kg, or 10 mg/kg.

In some embodiments, the anti-GITR antibody is administered to the human subject once every two weeks. In some embodiments, the anti-GITR antibody is administered to the human subject once every three weeks. In some embodiments, the anti-GITR antibody is administered to the human subject once every four weeks.

In some embodiments, the anti-GITR antibody is administered to the human subject intravenously once every two weeks at a dose between 10 to 750 mg. In some embodiments, the anti-GITR antibody is administered to the human subject intravenously once every two weeks at a dose of 300 mg. In some embodiments, the anti-GITR antibody is administered to the human subject intravenously once every two weeks at a dose of 600 mg.

In some embodiments, the anti-GITR antibody is administered to the human subject intravenously once every two weeks at a dose between 0.3 mg/kg to 10 mg/kg. In some embodiments, the anti-GITR antibody is administered to the human subject intravenously once every three weeks at a dose between 0.3 mg/kg to 10 mg/kg.

In some embodiments, the human subject has HNSCC, wherein the anti-GITR antibody is administered to the human subject intravenously once every two weeks at a dose of 300 mg, and wherein the anti-GITR antibody comprises a heavy chain comprising the amino acid sequence of SEQ ID NO:9 and a light chain comprising the amino acid sequence of SEQ ID NO:10.

In some embodiments, the human subject has HNSCC, wherein the anti-GITR antibody is administered to the human subject intravenously once every two weeks at a dose of 600 mg, and wherein the anti-GITR antibody comprises a heavy chain comprising the amino acid sequence of SEQ ID NO:9 and a light chain comprising the amino acid sequence of SEQ ID NO:10.

Aspects of the present disclosure provide a method for treating a cancer in a human subject in need thereof, the method comprising administering to the human subject an effective amount of an anti-GITR antibody and an anti-CTLA-4 antibody, wherein the anti-GITR antibody comprises a heavy chain variable region (VH) comprising a VH CDR1, a VH CDR2, and a VH CDR3, and a light chain variable region (VL) comprising a VL CDR1, a VL CDR2, and a VL CDR3, wherein the VH CDR1 comprises the amino acid sequence of DYAMY (SEQ ID NO:1), the VH CDR2 comprises the amino acid sequence of VIRTYSGDVTYNQKFKD (SEQ ID NO:2), the VH CDR3 comprises the amino acid sequence of SGTVRGFAY (SEQ ID NO:3), the VL CDR1 comprises the amino acid sequence of KSSQSLLNSGNQKNYLT (SEQ ID NO:4), the VL CDR2 comprises the amino acid sequence of WASTRES (SEQ ID NO:5), and the VL CDR3 comprises the amino acid sequence of QNDYSYPYT (SEQ ID NO:6).

In some embodiments, the method does not comprise administration of a multivalent autophagosome cancer vaccine to the human subject. In some embodiments, the method does not comprise administration of a cancer vaccine to the human subject.

In some embodiments, the human subject has an advanced solid tumor, advanced renal cell carcinoma, anal cancer, bladder cancer, brain cancer, breast cancer, cervical cancer, colorectal cancer, endometrial cancer, esophageal cancer, gastric cancer, gastrointestinal cancer, gastroesophageal cancer, glioblastoma, head and neck squamous cell carcinoma (HNSCC), hepatocellular carcinoma (HCC), glioma, kidney cancer, liver cancer, lung cancer, lymphoma, melanoma, Merkel cell carcinoma, mesothelioma, multiple myeloma, myeloma, neuroblastoma, non-small cell lung cancer (NSCLC), ovarian cancer, pancreatic cancer, prostate cancer, rectal cancer, renal cell carcinoma (RCC), sarcoma, skin cancer, small cell lung cancer (SCLC), soft-tissue sarcoma, thoracic cancer, thyroid cancer, triple-negative breast cancer (TNBC), urothelial cancer, uterine cancer, or a combination thereof.

In some embodiments, the human subject has an advanced solid tumor, breast cancer, cervical cancer, colorectal cancer, endometrial cancer, esophageal cancer, gastric cancer, gastrointestinal cancer, glioblastoma, head and neck squamous cell carcinoma (HNSCC), hepatocellular carcinoma (HCC), liver cancer, lung cancer, lymphoma, melanoma, Merkel cell carcinoma, mesothelioma, non-small cell lung cancer (NSCLC), ovarian cancer, pancreatic cancer, prostate cancer, renal cell carcinoma (RCC), small cell lung cancer (SCLC), soft-tissue sarcoma, thoracic cancer, triple-negative breast cancer (TNBC), urothelial cancer, or a combination thereof.

In some embodiments, the human subject has an unresectable cancer, a recurrent cancer, or a metastatic cancer.

In some embodiments, the human subject has head and neck squamous cell carcinoma (HNSCC). In some embodiments, the human subject has recurrent or metastatic HNSCC. In some embodiments, HNSCC tumor cells in a biological sample obtained from the human subject express GITR. In some embodiments, methods described herein comprise determining that HNSCC tumor cells in a biological sample obtained from the human subject express GITR. In some embodiments, at least 10% of HNSCC tumor cells in the biological sample obtained from the human subject express GITR.

In some embodiments, the human subject has undergone a prior anti-cancer therapy. In some embodiments, the prior anti-cancer therapy comprises, an immune checkpoint inhibitor, a chemotherapy, or a combination thereof. In some embodiments, the immune checkpoint inhibitor is an anti-PD-(L)1 inhibitor. In some embodiments, the human subject has progressed on or after prior systemic treatment. In some embodiments, the human subject has progressed on or after prior systemic anti-PD-(L)1 therapy or wherein the human subject has progressed on or after prior systemic anti-PD-1 therapy.

In some embodiments, the anti-GITR antibody is administered to the human subject intravenously at a dose between 0.1 to 1000 mg. In some embodiments, the anti-GITR antibody is administered to the human subject intravenously at a dose of 300 mg. In some embodiments, the anti-GITR antibody is administered to the human subject intravenously at a dose of 600 mg. In some embodiments, the anti-GITR antibody is administered to the human subject intravenously at a dose between 0.1 mg/kg to 10 mg/kg. In some embodiments, the anti-GITR antibody is administered to the human subject intravenously at a dose of 0.3 mg/kg, 1.0 mg/kg, 3.0 mg/kg, 5.0 mg/kg, or 10 mg/kg.

In some embodiments, the anti-CTLA-4 antibody is administered to the human subject intravenously at a dose between 100 to 1000 mg. In some embodiments, the anti-CTLA-4 antibody is administered to the human subject intravenously at a dose of 200 mg. In some embodiments, the anti-CTLA-4 antibody is administered to the human subject intravenously at a dose of 500 mg. In some embodiments, the anti-CTLA-4 antibody is administered to the human subject intravenously at a dose between 0.5 mg/kg to 5 mg/kg. In some embodiments, the anti-CTLA-4 antibody is administered to the human subject intravenously at a dose of 1 mg/kg. In some embodiments, the anti-CTLA-4 antibody is administered to the human subject intravenously at a dose of 3 mg/kg.

In some embodiments, the anti-GITR antibody and the anti-CTLA-4 antibody are administered simultaneously or sequentially.

In some embodiments, the anti-GITR antibody is administered to the human subject once every two weeks. In some embodiments, the anti-GITR antibody is administered to the human subject once every three weeks. In some embodiments, the anti-GITR antibody is administered to the human subject once every four weeks.

In some embodiments, the anti-CTLA-4 antibody is administered to the human subject once every two weeks. In some embodiments, the anti-CTLA-4 antibody is administered to the human subject once every three weeks. In some embodiments, the anti-CTLA-4 antibody is administered to the human subject once every six weeks.

In some embodiments, the anti-GITR antibody is administered to the human subject intravenously once every two weeks at a dose between 10 to 750 mg.

In some embodiments, the anti-GITR antibody is administered to the human subject intravenously once every two weeks at a dose of 300 mg. In some embodiments, the anti-GITR antibody is administered to the human subject intravenously once every two weeks at a dose of 600 mg. In some embodiments, the anti-GITR antibody is administered to the human subject intravenously once every two weeks at a dose between 0.3 mg/kg to 10 mg/kg. In some embodiments, the anti-GITR antibody is administered to the human subject intravenously once every three weeks at a dose between 0.3 mg/kg to 10 mg/kg.

In some embodiments, the anti-CTLA-4 antibody is administered to the human subject intravenously once every six weeks at a dose between 0.5 mg/kg to 5 mg/kg. In some embodiments, the anti-CTLA-4 antibody is administered to the human subject intravenously once every six weeks at a dose of 1 mg/kg. In some embodiments, the anti-CTLA-4 antibody is administered to the human subject intravenously once every six weeks at a dose of 3 mg/kg. In some embodiments, the anti-CTLA-4 antibody is administered to the human subject intravenously once every three weeks at a dose of 3 mg/kg. In some embodiments, the anti-CTLA-4 antibody is administered to the human subject intravenously once every six weeks at a dose between 100 to 1000 mg.

In some embodiments, the anti-GITR antibody is administered to the human subject intravenously once every two weeks at a dose of 300 mg, and wherein the anti-CTLA-4 antibody is administered to the human subject intravenously once every six weeks at a dose of 1 mg/kg. In some embodiments, the human subject has HNSCC or glioblastoma.

In some embodiments, the anti-GITR antibody is administered to the human subject intravenously once every two weeks at a dose of 600 mg, and wherein the anti-CTLA-4 antibody is administered to the human subject intravenously once every six weeks at a dose of 1 mg/kg. In some embodiments, the human subject has HNSCC.

In some embodiments, the anti-GITR antibody is administered between 12 to 28 days prior to administering the anti-CTLA-4 antibody.

In some embodiments, the anti-GITR antibody is administered to the human subject intravenously once every three weeks at a dose between 0.3 mg/kg to 10 mg/kg, and wherein the anti-CTLA-4 antibody is administered to the human subject intravenously once every six weeks at a dose between 0.5 mg/kg to 5 mg/kg.

In some embodiments, the human subject has cervical cancer, endometrial cancer, gastric cancer, esophageal cancer, hepatocellular carcinoma (HCC), melanoma, Merkel cell carcinoma, mesothelioma, non-small cell lung cancer (NSCLC), ovarian cancer, head and neck squamous cell carcinoma (HNSCC), small cell lung cancer (SCLC), renal cell carcinoma (RCC), triple-negative breast cancer (TNBC), urothelial cancer, or a combination thereof.

In some embodiments, the anti-GITR antibody is administered to the human subject intravenously once every two weeks at a dose between 0.3 mg/kg to 10 mg/kg, and wherein the anti-CTLA-4 antibody is administered to the human subject intravenously once every six weeks at a dose of 0.5 mg/kg to 5 mg/kg. In some embodiments, the human subject has cervical cancer, endometrial cancer, gastric cancer, esophageal cancer, hepatocellular carcinoma (HCC), melanoma, Merkel cell carcinoma, mesothelioma, non-small cell lung cancer (NSCLC), ovarian cancer, head and neck squamous cell carcinoma (HNSCC), small cell lung cancer (SCLC), renal cell carcinoma (RCC), triple-negative breast cancer (TNBC), urothelial cancer, or a combination thereof.

In some embodiments, the anti-GITR antibody is administered to the human subject intravenously once every two weeks at a dose of 300 mg, and wherein the anti-CTLA-4 antibody is administered to the human subject intravenously once every four weeks at a dose of 500 mg. In some embodiments, the human subject has glioblastoma.

In some embodiments, the anti-GITR antibody is administered to the human subject intravenously once every three weeks at a dose between 10 to 750 mg, and wherein the anti-CTLA-4 antibody is administered to the human subject intravenously once every three weeks at a dose between 100 to 300 mg. In some embodiments, the human subject has colorectal cancer, melanoma, ovarian cancer, breast cancer, cervical cancer, mesothelioma, pancreatic cancer, lymphoma, or a combination thereof.

In some embodiments, the anti-GITR antibody is administered to the human subject once every three weeks at a dose between 0.12 to 60 mg, and wherein the anti-CTLA-4 antibody is administered to the human subject once every three weeks at a dose of 200 mg. In some embodiments, the human subject has colorectal cancer, melanoma, renal cell carcinoma (RCC), ovarian cancer, breast cancer, cervical cancer, head and neck squamous cell carcinoma (HNSCC), hepatocellular carcinoma (HCC), non-small cell lung cancer (NSCLC), soft-tissue sarcoma, gastric cancer, gastrointestinal cancer, mesothelioma, pancreatic cancer, urothelial cancer, or a combination thereof.

In some embodiments, the anti-GITR antibody is administered to the human subject once every two weeks, and wherein the anti-CTLA-4 antibody is administered to the human subject once every six weeks. In some embodiments, the human subject has liver cancer, lung cancer, thoracic cancer, or a combination thereof.

In some embodiments, the VH of the anti-GITR antibody is at least 80% identical to the amino acid sequence of SEQ ID NO:7, and wherein the VL of the anti-GITR antibody is at least 80% identical to the amino acid sequence of SEQ ID NO:8.

In some embodiments, the VH of the anti-GITR antibody comprises the amino acid sequence of SEQ ID NO:7, and wherein the VL of the anti-GITR antibody comprises the amino acid sequence of SEQ ID NO:8.

In some embodiments, the anti-GITR antibody comprises a heavy chain that is at least 80% identical to the amino acid sequence of SEQ ID NO:9 and a light chain that is at least 80% identical to the amino acid sequence of SEQ ID NO:10.

In some embodiments, the anti-GITR antibody comprises a heavy chain comprising the amino acid sequence of SEQ ID NO:9 and a light chain comprising the amino acid sequence of SEQ ID NO:10.

In some embodiments, the anti-CTLA-4 antibody comprises a VH comprising a VH CDR1, a VH CDR2, and a VH CDR3, and a VL comprising a VL CDR1, a VL CDR2, and a VL CDR3, wherein the VH CDR1 comprises the amino acid sequence GFTFSSYT (SEQ ID NO:84), the VH CDR2 comprises the amino acid sequence TFISYDGNNK (SEQ ID NO:85), the VH CDR3 comprises the amino acid sequence ARTGWLGPFDY (SEQ ID NO:86), the VL CDR1 comprises the amino acid sequence QSVGSSY (SEQ ID NO:87), the VL CDR2 comprises the amino acid sequence GAF, and the VL CDR3 comprises the amino acid sequence QQYGSSPWT (SEQ ID NO:89).

In some embodiments, the VH of the anti-CTLA-4 antibody is at least 80% identical to the amino acid sequence of SEQ ID NO:90, and wherein the VL of the anti-CTLA-4 antibody is at least 80% identical to the amino acid sequence of SEQ ID NO:91. In some embodiments, the VH of the anti-CTLA-4 antibody comprises the amino acid sequence of SEQ ID NO:90, and wherein the VL of the anti-CTLA-4 antibody comprises the amino acid sequence of SEQ ID NO:91.

In some embodiments, the anti-CTLA-4 antibody comprises a heavy chain that is at least 80% identical to the amino acid sequence of SEQ ID NO:92 and a light chain that is at least 80% identical to the amino acid sequence of SEQ ID NO:93. In some embodiments, the anti-CTLA-4 antibody comprises a heavy chain comprising the amino acid sequence of SEQ ID NO:92 and a light chain comprising the amino acid sequence of SEQ ID NO:93.

In some embodiments, the CTLA-4 antibody comprises a VH comprising a VH CDR1, a VH CDR2, and a VH CDR3, and a VL comprising VL CDR1, a VL CDR2, and a VL CDR3, wherein the VH CDR1 comprises the amino acid sequence GFTFSSYG (SEQ ID NO:94), the VH CDR2 comprises the amino acid sequence AVIWYDGSNK (SEQ ID NO:95), the VH CDR3 comprises the amino acid sequence ARDPRGATLYYYYYGMDV (SEQ ID NO:96), the VL CDR1 comprises the amino acid sequence QSINSY (SEQ ID NO:97), the VL CDR2 comprises the amino acid sequence AAS, and the VL CDR3 comprising the amino acid sequence of QQYYSTPFT (SEQ ID NO:99).

In some embodiments, the VH of the anti-CTLA-4 antibody is at least 80% identical to the amino acid sequence of SEQ ID NO:100, and wherein the VL of the anti-CTLA-4 antibody is at least 80% identical to the amino acid sequence of SEQ ID NO:101. In some embodiments, the VH of the anti-CTLA-4 antibody comprises the amino acid sequence of SEQ ID NO:100, and wherein the VL of the anti-CTLA-4 antibody comprises the amino acid sequence of SEQ ID NO:101.

In some embodiments, the anti-CTLA-4 antibody comprises a heavy chain that is at least 80% identical to the amino acid sequence of SEQ ID NO:102 and a light chain that is at least 80% identical to the amino acid sequence of SEQ ID NO:103. In some embodiments, the anti-CTLA-4 antibody comprises a heavy chain comprising the amino acid sequence of SEQ ID NO:102 and a light chain comprising the amino acid sequence of SEQ ID NO:103.

In some embodiments, the human subject does not show one or more of the following features:
(a) prior treatment with an anticancer medication within the last 30 days;
(b) prior treatment with a tumor necrosis factor superfamily (TNFSF) agonist;
(c) significant worsening of clinical status;
(d) brain or central nervous system (CNS) metastases;
(e) active infection;
(f) chronic systemic steroid use;
(g) lung disease or pneumonitis; and
(h) vaccine use.

In some embodiments, the human subject has HNSCC, wherein the anti-GITR antibody comprises a heavy chain comprising the amino acid sequence of SEQ ID NO:9 and a light chain comprising the amino acid sequence of SEQ ID NO:10, and wherein the anti-CTLA-4 antibody comprises a heavy chain comprising the amino acid sequence of SEQ ID NO:92 and a light chain comprising the amino acid sequence of SEQ ID NO:93.

In some embodiments, the human subject has HNSCC, wherein the anti-GITR antibody is administered to the human subject intravenously once every two weeks at a dose of 300 mg, wherein the anti-CTLA-4 antibody is administered to the human subject intravenously once every six weeks at a dose of 1 mg/kg, wherein the anti-GITR antibody comprises a heavy chain comprising the amino acid sequence of SEQ ID NO:9 and a light chain comprising the amino acid sequence of SEQ ID NO:10, and wherein the anti-CTLA-4 antibody comprises a heavy chain comprising the amino acid sequence of SEQ ID NO:92 and a light chain comprising the amino acid sequence of SEQ ID NO:93.

In some embodiments, the human subject has HNSCC, wherein the anti-GITR antibody is administered to the human subject intravenously once every two weeks at a dose of 300 mg, wherein the anti-CTLA-4 antibody is administered to the human subject intravenously once every three weeks at a dose of 3 mg/kg, wherein the anti-GITR antibody comprises a heavy chain comprising the amino acid sequence of SEQ ID NO:9 and a light chain comprising the amino acid sequence of SEQ ID NO:10, and wherein the anti-CTLA-4 antibody comprises a heavy chain comprising the amino acid sequence of SEQ ID NO:92 and a light chain comprising the amino acid sequence of SEQ ID NO:93.

In some embodiments, the human subject has HNSCC, wherein the anti-GITR antibody is administered to the human subject intravenously once every two weeks at a dose of 300 mg, wherein the anti-CTLA-4 antibody is administered to the human subject intravenously once every six weeks at a dose of 3 mg/kg, wherein the anti-GITR antibody comprises a heavy chain comprising the amino acid sequence of SEQ ID NO:9 and a light chain comprising the amino acid sequence of SEQ ID NO:10, and wherein the anti-CTLA-4 antibody comprises a heavy chain comprising the amino acid sequence of SEQ ID NO:92 and a light chain comprising the amino acid sequence of SEQ ID NO:93.

In some embodiments, the human subject has cervical cancer, endometrial cancer, gastric cancer, esophageal cancer, hepatocellular carcinoma (HCC), melanoma, Merkel cell carcinoma, mesothelioma, non-small cell lung cancer (NSCLC), ovarian cancer, head and neck squamous cell carcinoma (HNSCC), small cell lung cancer (SCLC), renal cell carcinoma (RCC), triple-negative breast cancer (TNBC), urothelial cancer, or a combination thereof, wherein the anti-GITR antibody comprises a heavy chain comprising the amino acid sequence of SEQ ID NO:9 and a light chain comprising the amino acid sequence of SEQ ID NO:10, and wherein the anti-CTLA-4 antibody comprises a heavy chain comprising the amino acid sequence of SEQ ID NO:92 and a light chain comprising the amino acid sequence of SEQ ID NO:93.

In some embodiments, the human subject has cervical cancer, endometrial cancer, gastric cancer, esophageal cancer, hepatocellular carcinoma (HCC), melanoma, Merkel cell carcinoma, mesothelioma, non-small cell lung cancer (NSCLC), ovarian cancer, head and neck squamous cell carcinoma (HNSCC), small cell lung cancer (SCLC), renal cell carcinoma (RCC), triple-negative breast cancer (TNBC), urothelial cancer, or a combination thereof, wherein the anti-GITR antibody is administered to the human subject intravenously once every two weeks at a dose of 300 mg, wherein the anti-CTLA-4 antibody is administered to the human subject intravenously once every six weeks at a dose 1 mg/kg, wherein the anti-GITR antibody comprises a heavy chain comprising the amino acid sequence of SEQ ID NO:9 and a light chain comprising the amino acid sequence of SEQ ID NO:10, and wherein the anti-CTLA-4 antibody comprises a heavy chain comprising the amino acid sequence of SEQ ID NO:92 and a light chain comprising the amino acid sequence of SEQ ID NO:93.

In some embodiments, the human subject has cervical cancer, endometrial cancer, gastric cancer, esophageal cancer, hepatocellular carcinoma (HCC), melanoma, Merkel cell carcinoma, mesothelioma, non-small cell lung cancer (NSCLC), ovarian cancer, head and neck squamous cell carcinoma (HNSCC), small cell lung cancer (SCLC), renal cell carcinoma (RCC), triple-negative breast cancer (TNBC), urothelial cancer, or a combination thereof, wherein the anti-GITR antibody is administered to the human subject intravenously once every two weeks at a dose of 300 mg, wherein the anti-CTLA-4 antibody is administered to the human subject intravenously once every three weeks at a dose of 3 mg/kg, wherein the anti-GITR antibody comprises a heavy chain comprising the amino acid sequence of SEQ ID NO:9 and a light chain comprising the amino acid sequence of SEQ ID NO:10, and wherein the anti-CTLA-4 antibody comprises a heavy chain comprising the amino acid sequence of SEQ ID NO:92 and a light chain comprising the amino acid sequence of SEQ ID NO:93.

In some embodiments, the human subject has cervical cancer, endometrial cancer, gastric cancer, esophageal cancer, hepatocellular carcinoma (HCC), melanoma, Merkel cell carcinoma, mesothelioma, non-small cell lung cancer (NSCLC), ovarian cancer, head and neck squamous cell carcinoma (HNSCC), small cell lung cancer (SCLC), renal cell carcinoma (RCC), triple-negative breast cancer (TNBC), urothelial cancer, or a combination thereof, wherein the anti-GITR antibody is administered to the human subject intravenously once every three weeks at a dose of 300 mg, wherein the anti-CTLA-4 antibody is administered to the human subject intravenously once every six weeks at a dose of 3 mg/kg, wherein the anti-GITR antibody comprises a heavy chain comprising the amino acid sequence of SEQ ID NO:9 and a light chain comprising the amino acid sequence of SEQ ID NO:10, and wherein the anti-CTLA-4 antibody comprises a heavy chain comprising the amino acid sequence of SEQ ID NO:92 and a light chain comprising the amino acid sequence of SEQ ID NO:93.

In some embodiments, the human subject has cervical cancer, endometrial cancer, gastric cancer, esophageal cancer, hepatocellular carcinoma (HCC), melanoma, Merkel cell carcinoma, mesothelioma, non-small cell lung cancer (NSCLC), ovarian cancer, head and neck squamous cell carcinoma (HNSCC), small cell lung cancer (SCLC), renal cell carcinoma (RCC), triple-negative breast cancer (TNBC), urothelial cancer, or a combination thereof, wherein the anti-GITR antibody is administered to the human subject intravenously once every two weeks at a dose between 0.3 mg/kg to 10 mg/kg, wherein the anti-CTLA-4 antibody is administered to the human subject intravenously once every six weeks at a dose between 0.5 mg/kg to 5 mg/kg, wherein the anti-GITR antibody comprises a heavy chain comprising the amino acid sequence of SEQ ID NO:9 and a light chain comprising the amino acid sequence of SEQ ID NO:10, and wherein the anti-CTLA-4 antibody comprises a heavy chain comprising the amino acid sequence of SEQ ID NO:92 and a light chain comprising the amino acid sequence of SEQ ID NO:93.

In some embodiments, the human subject is at least 18 years of age.

### DETAILED DESCRIPTION

The present disclosure provides, in part, methods of treating cancer by administering a combination of an anti-GITR antibody and a PD-1 inhibitor (e.g., an anti-PD-1 antibody), a PD-L1 inhibitor (e.g., an anti-PD-L1 antibody) or a CTLA-4 inhibitor (e.g., a CTLA-4 antibody). While not wishing to be bound by theory, it is believed that such a combination provides improved treatment over previous monotherapies because targeting alternative immune pathways may result in enhanced activation of the immune response against the tumor, thereby improving patient outcomes. Non-limiting examples of combination treatments described herein include a combination of an anti-GITR antibody and an anti-PD-1 antibody, a combination of an anti-GITR antibody and an anti-PD-L1 antibody, a combination of an anti-GITR antibody and anti-CTLA-4 antibody, and a combination of an anti-GITR antibody, an anti-PD-1 antibody, and a CTLA-4 antibody.

Monotherapy treatments have demonstrated limited therapeutic benefits to patients. For example, in clinical trials, anti-GITR monotherapy treatments have demonstrated manageable safety profiles but limited antitumor activity in patients with advanced cancers. Similarly, anti-PD-1 monotherapy has been used as first-line therapy for recurrent or metastatic head and neck squamous cell carcinoma (HNSCC), however, the majority of patients do not respond or acquire resistance to treatment over time. Thus, the combination therapies described herein can benefit patients with advanced cancer and patients who did not respond or develop resistance to monotherapy treatment.

### GITR and Anti-GITR Antibodies

Glucocorticoid-induced TNFR-related (GITR) (also known as CD357 or TNFRSF18) is a member of the TNF receptor superfamily which is expressed constitutively at high levels on regulatory T cells (Tregs) and is inducible on effector T cells. Engagement of GITR by its ligand, GITRL, mainly expressed by antigen presenting cells and endothelial cells, provides a costimulatory signal to both CD4⁺ and CD8⁺ T cells, enhancing proliferation and effector function, particularly in the setting of suboptimal TCR stimulation.

The GITR gene codes for at least 3 alternatively spliced products. Isoform 1 is a type I membrane protein that is 241 amino acids in length. Isoform 2 is a secreted, soluble form of human GITR that is 255 amino acids in length. Isoform 3 is a type I membrane protein that is 234 amino acids in length. Exemplary amino acid sequences of isoforms 1-3 of human GITR are provided below.

An exemplary amino acid sequence of isoform 1 of the human GITR protein (amino acids 1-241 of GenBank Accession No. NP_004186) is:

(SEQ ID NO: 104)
MAQHGAMGAFRALCGLALLCALSLGQRPTGGPGCGPGRLLLGTGTDARC
CRVHTTRCCRDYPGEECCSEWDCMCVQPEFHCGDPCCTTCRHHPCPPGQ
GVQSQGKFSFGFQCIDCASGTFSGGHEGHCKPWTDCTQFGFLTVFPGNK
THNAVCVPGSPPAEPLGWLTVVLLAVAACVLLLTSAQLGLHIWQLRSQC
MWPRETQLLLEVPPSTEDARSCQFPEEERGERSAEEKGRLGDLWV.

An exemplary amino acid sequence of isoform 2 of the human GITR protein (amino acids 1-255 of GenBank Accession No. NP_683699) is:

(SEQ ID NO: 105)
MAQHGAMGAFRALCGLALLCALSLGQRPTGGPGCGPGRLLLGTGTDARC
CRVHTTRCCRDYPGEECCSEWDCMCVQPEFHCGDPCCTTCRHHPCPPGQ
GVQSQGKFSFGFQCIDCASGTFSGGHEGHCKPWTDCCWRCRRRPKTPEA
ASSPRKSGASDRQRRRGGWETCGCEPGRPPGPPTAASPSPGAPQAAGAL
RSALGRALLPWQQKWVQEGGSDQRPGPCSSAAAAGPCRRERETQSWPPS
SLAGPDGVGS.

An exemplary amino acid sequence of isoform 3 of the human GITR protein (amino acids 1-234 of GenBank Accession No. NP_683700) is:

(SEQ ID NO: 106)
MAQHGAMGAFRALCGLALLCALSLGQRPTGGPGCGPGRLLLGTGTDARC
CRVHTTRCCRDYPGEECCSEWDCMCVQPEFHCGDPCCTTCRHHPCPPGQ
GVQSQGKFSFGFQCIDCASGTFSGGHEGHCKPWTDCTQFGFLTVFPGNK
THNAVCVPGSPPAEPLGWLTVVLLAVAACVLLLTSAQLGLHIWQLRKTQ
LLLEVPPSTEDARSCQFPEEERGERSAEEKGRLGDLWV.

This disclosure provides anti-GITR antibodies that are useful in treating a cancer.

In some embodiments, the anti-GITR antibody comprises a heavy chain variable region (VH) comprising a VH CDR1 comprising the amino acid sequence of DYAMY (SEQ ID NO:1), a VH CDR2 comprising the amino acid sequence of VIRTYSGDVTYNQKFKD (SEQ ID NO:2), and a VH CDR3 comprising the amino acid sequence of SGTVRGFAY (SEQ ID NO:3), and a light chain variable region (VL) comprising a VL CDR1 comprising KSSQSLLNSGNQKNYLT (SEQ ID NO:4), a VL CDR2 comprising the amino acid sequence of WASTRES (SEQ ID NO:5), and a VL CDR3 comprising the amino acid sequence of QNDYSYPYT (SEQ ID NO:6).

In some embodiments, the anti-GITR antibody comprises a heavy chain variable region (VH) comprising a VH CDR1 comprising the amino acid sequence of DYAMY (SEQ ID NO:1), or a variant thereof comprising 1, 2, 3, or 4 amino acid substitutions; a VH CDR2 comprising the amino acid sequence of VIRTYSGDVTYNQKFKD (SEQ ID NO:2), or a variant thereof comprising 1, 2, 3, or 4 amino acid substitutions; and a VH CDR3 comprising the amino acid sequence of SGTVRGFAY (SEQ ID NO:3), or a variant thereof comprising 1, 2, 3, or 4 amino acid substitutions; and a light chain variable region (VL) comprising a VL CDR1 comprising KSSQSLLNSGNQKNYLT (SEQ ID NO:4), or a variant thereof comprising 1, 2, 3, or 4 amino acid substitutions; a VL CDR2 comprising the amino acid sequence of WASTRES (SEQ ID NO:5), or a variant thereof comprising 1, 2, 3, or 4 amino acid substitutions; and a VL CDR3 comprising the amino acid sequence of QNDYSYPYT (SEQ ID NO:6), or a variant thereof comprising 1, 2, 3, or 4 amino acid substitutions.

In some embodiments, the anti-GITR antibody comprises a heavy chain variable region (VH) comprising a VH CDR1 comprising the amino acid sequence of DYAMY (SEQ ID NO:1), a VH CDR2 comprising the amino acid sequence of VIRTYSGDVTYNQKFKD (SEQ ID NO:2), and a VH CDR3 comprising the amino acid sequence of SGTVRGFAY (SEQ ID NO:3), and which has at least 75%, 80%, 85%, 90%, 95%, 96%, 97%, 98%, 99%, or 100% identity to the amino acid sequence of SEQ ID NO:7, and a light chain variable region (VL) comprising a VL CDR1 comprising KSSQSLLNSGNQKNYLT (SEQ ID NO:4), a VL CDR2 comprising the amino acid sequence of WASTRES (SEQ ID NO:5), and a VL CDR3 comprising the amino acid sequence of QNDYSYPYT (SEQ ID NO:6), and which has at least 75%, 80%, 85%, 90%, 95%, 96%, 97%, 98%, 99%, or 100% identity to the amino acid sequence of SEQ ID NO:8.

In some embodiments, the anti-GITR antibody comprises a heavy chain variable region with one or more (e.g., 1, 2, or 3) substitutions, deletions, or insertions in the amino acid sequence of SEQ TD NO:7, and a light chain variable region with one or more (e.g., 1, 2, or 3) substitutions, deletions, or insertions in the amino acid sequence of SEQ ID NO:8.

In some embodiments, the anti-GITR antibody comprises a heavy chain variable region comprising the amino acid sequence of SEQ ID NO:7 and a light chain variable region comprising the amino acid sequence of SEQ TD NOS8 In some embodiments, the anti-GITR antibody comprises a heavy chain that is at least 75%, 80%, 85%, 90%, 95%, 96%, 97%, 98%, 99% or 1000% identical to the amino acid sequence of SEQ ID NO:9 and a light chain that is at least 75%, 80%, 85%, 90%, 95%, 96%, 97%, 98%, 99%, or 100% identical to the amino acid sequence of SEQ TD NO:10.

In some embodiments, the anti-GITR antibody comprises a heavy chain comprising the amino acid sequence of SEQ TD NO:9 and a light chain comprising the amino acid sequence of SEQ TD NO: 10.

Non-limiting examples of anti-GITR antibodies that can be used in methods described herein are provided in U.S. Pat. No. 10,280,226, which is incorporated herein by reference in its entirety. The amino acid sequence of an exemplary anti-GITR antibody, Anti-GITR Antibody A, is described in Table 1.

TABLE 1
Anti-GITR Antibody A Sequences

| Antibody | |
|---|---|
| Feature | Sequences |
| VH CDR1 | DYAMY (SEQ ID NO: 1) |
| VH CDR2 | VIRTYSGDVTYNQKFKD (SEQ ID NO: 2) |
| VH CDR3 | SGTVRGFAY (SEQ ID NO: 3) |
| VL CDR1 | KSSQSLLNSGNQKNYLT (SEQ ID NO: 4) |
| VL CDR2 | WASTRES (SEQ ID NO: 5) |
| VL CDR3 | QNDYSYPYT (SEQ ID NO: 6) |
| VH | QVQLVQSGAEVKKPGASVKVSCKGSGYTFTDYAMYWVRQAPGQGLEWIGVIRTYSG |
| | DVTYNQKFKDRATMTVDKSISTAYMELSRLRSDDTAVYYCAKSGTVRGFAYWGQGT |
| | LVTVSS (SEQ ID NO: 7) |
| VL | DIVMTQSPDSLAVSLGERATINCKSSQSLLNSGNQKNYLTWYQQKPGQPPKLLIYW |
| | ASTRESGVPDRFSGSGSGTDFTLTISSLQAEDVAVYHCQNDYSYPYTFGQGTKLEI |
| | K (SEQ ID NO: 8) |
| HC | QVQLVQSGAEVKKPGASVKVSCKGSGYTFTDYAMYWVRQAPGQGLEWIGVIRTYSG |
| | DVTYNQKFKDRATMTVDKSISTAYMELSRLRSDDTAVYYCAKSGTVRGFAYWGQGT |
| | LVTVSSASTKGPSVFPLAPSSKSTSGGTAALGCLVKDYFPEPVTVSWNSGALTSGV |
| | HTFPAVLQSSGLYSLSSVVTVPSSSLGTQTYICNVNHKPSNTKVDKRVEPKSCDKT |
| | HTCPPCPAPELLGGPSVFLFPPKPKDTLMISRTPEVTCVVVDVSHEDPEVKFNWYV |
| | DGVEVHNAKTKPREEQYNSTYRVVSVLTVLHQDWLNGKEYKCKVSNKALPAPIEKT |
| | ISKAKGQPREPQVYTLPPSREEMTKNQVSLTCLVKGFYPSDIAVEWESNGQPENNY |
| | KTTPPVLDSDGSFFLYSKLTVDKSRWQQGNVFSCSVMHEALHNHYTQKSLSLSPG |
| | (SEQ ID NO: 9) |
| LC | DIVMTQSPDSLAVSLGERATINCKSSQSLLNSGNQKNYLTWYQQKPGQPPKLLIYW |
| | ASTRESGVPDRFSGSGSGTDFTLTISSLQAEDVAVYHCQNDYSYPYTFGQGTKLEI |
| | KRTVAAPSVFIFPPSDEQLKSGTASVVCLLNNFYPREAKVQWKVDNALQSGNSQES |
| | VTEQDSKDSTYSLSSTLTLSKADYEKHKVYACEVTHQGLSSPVTKSFNRGEC |
| | (SEQ ID NO: 10) |

### Inhibitors of PD-1 and PD-L1

Programmed Death-1 receptor (PD-1) (also known as CD279) is type I transmembrane protein belonging to the CD28 family of immune regulatory receptors. PD-1 is expressed on activated T cells and B cells and plays a critical role in regulating immune responses to tumor antigens and autoantigens. Engagement of PD-1 by either of its ligands, PD-L1 or PD-L2, on an adjacent cell inhibits TCR signaling and TCR-mediated proliferation, transcriptional activation, and cytokine production.

The amino acid sequence of the mature human PD-1 protein (amino acids 1-288 of GenBank Accession No. NP_005009.2) is:

(SEQ ID NO: 81)
MQIPQAPWPVVWAVLQLGWRPGWELDSPDRPWNPPTFSPALLVVTEGDN
ATFTCSFSNTSESFVLNWYRMSPSNQTDKLAAFPEDRSQPGQDCRFRVT
QLPNGRDFHMSVVRARRNDSGTYLCGAISLAPKAQIKESLRAELRVTER
RAEVPTAHPSPSPRPAGQFQTLVVGVVGGLLGSLVLLVWVLAVICSRAA
RGTIGARRTGQPLKEDPSAVPVFSVDYGELDFQWREKTPEPPVPCVPEQ
TEYATIVFPSGMGTSSPARRGSADGPRSAQPLRPEDGHCSWPL.

The amino acid sequence of the mature human PD-L1 protein (amino acids 1-290 of GenBank Accession No. NP_054862.1) is:

(SEQ ID NO: 82)
MRIFAVFIFMTYWHLLNAFTVTVPKDLYVVEYGSNMTIECKFPVEKQLD
LAALIVYWEMEDKNIIQFVHGEEDLKVQHSSYRQRARLLKDQLSLGNAA
LQITDVKLQDAGVYRCMISYGGADYKRITVKVNAPYNKINQRILVVDPV
TSEHELTCQAEGYPKAEVIWTSSDHQVLSGKTTTTNSKREEKLENVTST
LRINTTTNEIFYCTFRRLDPEENHTAELVIPELPLAHPPNERTHLVILG
AILLCLGVALTFIFRLRKGRMMDVKKCGIQDTNSKKQSDTHLEET.

This disclosure provides PD-1 inhibitors (e.g., anti-PD-1 antibodies) and PD-L1 inhibitors (e.g., anti-PD-L1 antibodies) that are useful in treating a cancer.

The terms “PD-1 inhibitor” and “PD-L1 inhibitor” as used herein, refer to a molecule that binds to PD-1 or PD-L1 (e.g., a small molecule or a biological molecule) and blocks, inhibits, reduces (including significantly), or interferes with PD-1 biological activity or PD-L1 biological activity, respectively.

In some embodiments, a PD-1 inhibitor is a molecule that inhibits or disrupts PD-1 itself (e.g., human PD-1), a biological activity of PD-1 (e.g., including but not limited to its ability to mediate any aspect of cancer), or the consequences of the biological activity to any meaningful degree, e.g., at least 10%, at least 20%, at least 30%, at least 40%, at least 50%, at least 60%, at least 70%, at least 80%, at least 90%, or more.

In some embodiments, a PD-L1 inhibitor is a molecule that inhibits or disrupts PD-L1 itself (e.g., human PD-L1), a biological activity of PD-L1 (e.g., including but not limited to its ability to mediate any aspect of cancer), or the consequences of the biological activity to any meaningful degree, e.g., at least 10%, at least 20%, at least 30%, at least 40%, at least 50%, at least 60%, at least 70%, at least 80%, at least 90%, or more.

Non-limiting examples of a PD-1 inhibitor for use in the methods described herein include a small molecule, anti-PD-1 antibody, or a peptide that inhibits PD-1 (e.g., a peptide aptamer, a PD-1 structural analog).

In some examples, the PD-1 inhibitor comprises an anti-PD-1 antibody.

In some embodiments, the anti-PD-1 antibody comprises a heavy chain variable region comprising a VH CDR1 comprising the amino acid sequence of SYWMN (SEQ ID NO:11), a VH CDR2 comprising the amino acid sequence of VIHPSDSETWLDQKFKD (SEQ ID NO:12), and a VH CDR3 comprising the amino acid sequence of EHYGTSPFAY (SEQ ID NO:13), and a light chain variable region comprising a VL CDR1 comprising RASESVDNYGMSFMNW (SEQ ID NO:14), a VL CDR2 comprising the amino acid sequence of AASNQGS (SEQ ID NO:15), and a VL CDR3 comprising the amino acid sequence of QQSKEVPYT (SEQ ID NO:16).

In some embodiments, the anti-PD-1 antibody comprises a heavy chain variable region comprising a VH CDR1 comprising the amino acid sequence of SYWMN (SEQ ID NO:11), or a variant thereof comprising 1, 2, 3, or 4 amino acid substitutions; a VH CDR2 comprising the amino acid sequence of VIHPSDSETWLDQKFKD (SEQ ID NO:12), or a variant thereof comprising 1, 2, 3, or 4 amino acid substitutions; and a VH CDR3 comprising the amino acid sequence of EHYGTSPFAY (SEQ ID NO:13), or a variant thereof comprising 1, 2, 3, or 4 amino acid substitutions; and a light chain variable region comprising a VL CDR1 comprising RASESVDNYGMSFMNW (SEQ ID NO:14), or a variant thereof comprising 1, 2, 3, or 4 amino acid substitutions; a VL CDR2 comprising the amino acid sequence of AASNQGS (SEQ ID NO:15), or a variant thereof comprising 1, 2, 3, or 4 amino acid substitutions; and a VL CDR3 comprising the amino acid sequence of QQSKEVPYT (SEQ ID NO:16), or a variant thereof comprising 1, 2, 3, or 4 amino acid substitutions.

In some embodiments, the anti-PD-1 antibody comprises a heavy chain variable region comprising a VH CDR1 comprising the amino acid sequence of SYWMN (SEQ ID NO:11), a VH CDR2 comprising the amino acid sequence of VIHPSDSETWLDQKFKD (SEQ ID NO:12), and a VH CDR3 comprising the amino acid sequence of EHYGTSPFAY (SEQ ID NO:13), and which has at least 75%, 80%, 85%, 90%, 95%, 96%, 97%, 98%, 99%, or 100% identity to the amino acid sequence of SEQ ID NO:17, and a light chain variable region comprising a VL CDR1 comprising RASESVDNYGMSFMNW (SEQ ID NO:14), a VL CDR2 comprising the amino acid sequence of AASNQGS (SEQ ID NO:15), and a VL CDR3 comprising the amino acid sequence of QQSKEVPYT (SEQ ID NO:16), and which has at least 75%, 80%, 85%, 90%, 95%, 96%, 97%, 98%, 99%, or 100% identity to the amino acid sequence of SEQ ID NO:18.

In some embodiments, the anti-PD-1 antibody comprises a heavy chain variable region with one or more (e.g., 1, 2, or 3) substitutions, deletions, or insertions in the amino acid sequence of SEQ ID NO:17, and a light chain variable region with one or more (e.g., 1, 2, or 3) substitutions, deletions, or insertions in the amino acid sequence of SEQ ID NO: 18.

In some embodiments, the anti-PD-1 antibody comprises a heavy chain variable region comprising the amino acid sequence of SEQ ID NO: 17 and a light chain variable region comprising the amino acid sequence of SEQ ID NO:18.

In some embodiments, the anti-PD-1 antibody comprises a heavy chain that is at least 75%, 80%, 85%, 90%, 95%, 96%, 97%, 98%, 99%, or 100% identical to the amino acid sequence of SEQ ID NO:19 and a light chain that is at least 75%, 80%, 85%, 90%, 95%, 96%, 97%, 98%, 99%, or 100% identical to the amino acid sequence of SEQ ID NO:20.

In some embodiments, the anti-PD-1 antibody comprises a heavy chain comprising the amino acid sequence of SEQ ID NO: 19 and a light chain comprising the amino acid sequence of SEQ ID NO:20.

In some embodiments, the anti-PD-1 antibody comprises a VH comprising a VH CDR1, a VH CDR2, and a VH CDR3, and a VL comprising a VL CDR1, a VL CDR2, and a VL CDR3, wherein the VH CDR1 comprises the amino acid sequence NYYMY (SEQ ID NO:21), the VH CDR2 comprises the amino acid sequence GINPSNGGTNFNEKFKN (SEQ ID NO:22), the VH CDR3 comprises the amino acid sequence RDYRFDMGFDY (SEQ ID NO:23), the VL CDR1 comprises the amino acid sequence RASKGVSTSGYSYLH (SEQ ID NO:24), the VL CDR2 comprises the amino acid sequence LASYLES (SEQ ID NO:25), and the VL CDR3 comprises the amino acid sequence QHSRDLPLT (SEQ ID NO:26).

In some embodiments, the anti-PD-1 antibody comprises a VH comprising a VH CDR1, a VH CDR2, and a VH CDR3, and a VL comprising a VL CDR1, a VL CDR2, and a VL CDR3, wherein the VH CDR1 comprises the amino acid sequence NYYMY (SEQ ID NO:21), or a variant thereof comprising 1, 2, 3, or 4 amino acid substitutions; the VH CDR2 comprises the amino acid sequence GINPSNGGTNFNEKFKN (SEQ ID NO:22), or a variant thereof comprising 1, 2, 3, or 4 amino acid substitutions; the VH CDR3 comprises the amino acid sequence RDYRFDMGFDY (SEQ ID NO:23), or a variant thereof comprising 1, 2, 3, or 4 amino acid substitutions; the VL CDR1 comprises the amino acid sequence RASKGVSTSGYSYLH (SEQ ID NO:24), or a variant thereof comprising 1, 2, 3, or 4 amino acid substitutions; the VL CDR2 comprises the amino acid sequence LASYLES (SEQ ID NO:25), or a variant thereof comprising 1, 2, 3, or 4 amino acid substitutions; and the VL CDR3 comprises the amino acid sequence QHSRDLPLT (SEQ ID NO:26), or a variant thereof comprising 1, 2, 3, or 4 amino acid substitutions.

In some embodiments, the anti-PD-1 antibody comprises a VH comprising a VH CDR1, a VH CDR2, and a VH CDR3, and a VL comprising a VL CDR1, a VL CDR2, and a VL CDR3, wherein the VH CDR1 comprises the amino acid sequence NYYMY (SEQ ID NO:21), the VH CDR2 comprises the amino acid sequence GINPSNGGTNFNEKFKN (SEQ ID NO:22), the VH CDR3 comprises the amino acid sequence RDYRFDMGFDY (SEQ ID NO:23), and which has at least 75%, 80%, 85%, 90%, 95%, 96%, 97%, 98%, 99%, or 100% identity to the amino acid sequence of SEQ ID NO:27, and the VL CDR1 comprises the amino acid sequence RASKGVSTSGYSYLH (SEQ ID NO:24), the VL CDR2 comprises the amino acid sequence LASYLES (SEQ ID NO:25), and the VL CDR3 comprises the amino acid sequence QHSRDLPLT (SEQ ID NO:26), and which has at least 75%, 80%, 85%, 90%, 95%, 96%, 97%, 98%, 99%, or 100% identity to the amino acid sequence of SEQ ID NO:28.

In some embodiments, the anti-PD-1 antibody comprises a heavy chain variable region with one or more (e.g., 1, 2, or 3) substitutions, deletions, or insertions in the amino acid sequence of SEQ ID NO:27, and a light chain variable region with one or more (e.g., 1, 2, or 3) substitutions, deletions, or insertions in the amino acid sequence of SEQ ID NO:28.

In some embodiments, the anti-PD-1 antibody comprises a heavy chain variable region comprising the amino acid sequence of SEQ ID NO:27 and a light chain variable region comprising the amino acid sequence of SEQ ID NO:28.

In some embodiments, the anti-PD-1 antibody comprises a heavy chain that is at least 75%, 80%, 85%, 90%, 95%, 96%, 97%, 98%, 99%, or 100% identical to the amino acid sequence of SEQ ID NO:29 and a light chain that is at least 75%, 80%, 85%, 90%, 95%, 96%, 97%, 98%, 99%, or 100% identical to the amino acid sequence of SEQ ID NO:30.

In some embodiments, the anti-PD-1 antibody comprises a heavy chain comprising the amino acid sequence of SEQ ID NO:29 and a light chain comprising the amino acid sequence of SEQ ID NO:30.

In some embodiments, the anti-PD-1 antibody comprises the anti-PD-1 antibody comprises a VH comprising a VH CDR1, a VH CDR2, and a VH CDR3, and a VL comprising a VL CDR1, a VL CDR2, and a VL CDR3, wherein the VH CDR1 comprises the amino acid sequence NSGMH (SEQ ID NO:31), the VH CDR2 comprises the amino acid sequence VIWYDGSKRYYADSVKG (SEQ ID NO:32), the VH CDR3 comprises the amino acid sequence NDDY (SEQ ID NO:33), the VL CDR1 comprises the amino acid sequence RASQSVSSYLA (SEQ ID NO:34), the VL CDR2 comprises the amino acid sequence DASNRAT (SEQ ID NO:35), and the VL CDR3 comprises the amino acid sequence QQSSNWPRT (SEQ ID NO:36).

In some embodiments, the anti-PD-1 antibody comprises the anti-PD-1 antibody comprises a VH comprising a VH CDR1, a VH CDR2, and a VH CDR3, and a VL comprising a VL CDR1, a VL CDR2, and a VL CDR3, wherein the VH CDR1 comprises the amino acid sequence NSGMH (SEQ ID NO:31), or a variant thereof comprising 1, 2, 3, or 4 amino acid substitutions; the VH CDR2 comprises the amino acid sequence VIWYDGSKRYYADSVKG (SEQ ID NO:32), or a variant thereof comprising 1, 2, 3, or 4 amino acid substitutions; the VH CDR3 comprises the amino acid sequence NDDY (SEQ ID NO:33), or a variant thereof comprising 1, 2, 3, or 4 amino acid substitutions; the VL CDR1 comprises the amino acid sequence RASQSVSSYLA (SEQ ID NO:34), or a variant thereof comprising 1, 2, 3, or 4 amino acid substitutions; the VL CDR2 comprises the amino acid sequence DASNRAT (SEQ ID NO:35), or a variant thereof comprising 1, 2, 3, or 4 amino acid substitutions; and the VL CDR3 comprises the amino acid sequence QQSSNWPRT (SEQ ID NO:36), or a variant thereof comprising 1, 2, 3, or 4 amino acid substitutions.

In some embodiments, the anti-PD-1 antibody comprises the anti-PD-1 antibody comprises a VH comprising a VH CDR1, a VH CDR2, and a VH CDR3, and a VL comprising a VL CDR1, a VL CDR2, and a VL CDR3, wherein the VH CDR1 comprises the amino acid sequence NSGMH (SEQ ID NO:31), the VH CDR2 comprises the amino acid sequence VIWYDGSKRYYADSVKG (SEQ ID NO:32), the VH CDR3 comprises the amino acid sequence NDDY (SEQ ID NO:33), and which has at least 75%, 80%, 85%, 90%, 95%, 96%, 97%, 98%, 99%, or 100% identity to the amino acid sequence of SEQ ID NO:37, the VL CDR1 comprises the amino acid sequence RASQSVSSYLA (SEQ ID NO:34), the VL CDR2 comprises the amino acid sequence DASNRAT (SEQ ID NO:35), and the VL CDR3 comprises the amino acid sequence QQSSNWPRT (SEQ ID NO:36), and which has at least 75%, 80%, 85%, 90%, 95%, 96%, 97%, 98%, 99%, or 100% identity to the amino acid sequence of SEQ ID NO:38.

In some embodiments, the anti-PD-1 antibody comprises a heavy chain variable region with one or more (e.g., 1, 2, or 3) substitutions, deletions, or insertions in the amino acid sequence of SEQ ID NO:37, and a light chain variable region with one or more (e.g., 1, 2, or 3) substitutions, deletions, or insertions in the amino acid sequence of SEQ ID NO:38.

In some embodiments, the anti-PD-1 antibody comprises a heavy chain variable region comprising the amino acid sequence of SEQ ID NO:37 and a light chain variable region comprising the amino acid sequence of SEQ ID NO:38.

In some embodiments, the anti-PD-1 antibody comprises a heavy chain that is at least 75%, 80%, 85%, 90%, 95%, 96%, 97%, 98%, 99%, or 100% identical to the amino acid sequence of SEQ ID NO:39 and a light chain that is at least 75%, 80%, 85%, 90%, 95%, 96%, 97%, 98%, 99%, or 100% identical to the amino acid sequence of SEQ ID NO:40.

In some embodiments, the anti-PD-1 antibody comprises a heavy chain comprising the amino acid sequence of SEQ ID NO:39 and a light chain comprising the amino acid sequence of SEQ ID NO:40.

In some embodiments, the anti-PD-1 antibody comprises a VH comprising a VH CDR1, a VH CDR2, and a VH CDR3, and a VL comprising a VL CDR1, a VL CDR2, and a VL CDR3, wherein the VH CDR1 comprises the amino acid sequence NSGMH (SEQ ID NO:41), the VH CDR2 comprises the amino acid sequence VIWYDGSKRYYADSVKG (SEQ ID NO:42), the VH CDR3 comprises the amino acid sequence NDDY (SEQ ID NO:43), the VL CDR1 comprises the amino acid sequence RASQSVSSYLA (SEQ ID NO:44), the VL CDR2 comprises the amino acid sequence DASNRAT (SEQ ID NO:45), and the VL CDR3 comprises the amino acid sequence QQSSNWPRT (SEQ ID NO:46).

In some embodiments, the anti-PD-1 antibody comprises a VH comprising a VH CDR1, a VH CDR2, and a VH CDR3, and a VL comprising a VL CDR1, a VL CDR2, and a VL CDR3, wherein the VH CDR1 comprises the amino acid sequence NSGMH (SEQ ID NO:41), or a variant thereof comprising 1, 2, 3, or 4 amino acid substitutions; the VH CDR2 comprises the amino acid sequence VIWYDGSKRYYADSVKG (SEQ ID NO:42), or a variant thereof comprising 1, 2, 3, or 4 amino acid substitutions; the VH CDR3 comprises the amino acid sequence NDDY (SEQ ID NO:43), or a variant thereof comprising 1, 2, 3, or 4 amino acid substitutions; the VL CDR1 comprises the amino acid sequence RASQSVSSYLA (SEQ ID NO:44), or a variant thereof comprising 1, 2, 3, or 4 amino acid substitutions; the VL CDR2 comprises the amino acid sequence DASNRAT (SEQ ID NO:45), or a variant thereof comprising 1, 2, 3, or 4 amino acid substitutions; and the VL CDR3 comprises the amino acid sequence QQSSNWPRT (SEQ ID NO:46), or a variant thereof comprising 1, 2, 3, or 4 amino acid substitutions.

In some embodiments, the anti-PD-1 antibody comprises a VH comprising a VH CDR1, a VH CDR2, and a VH CDR3, and a VL comprising a VL CDR1, a VL CDR2, and a VL CDR3, wherein the VH CDR1 comprises the amino acid sequence NSGMH (SEQ ID NO:41), the VH CDR2 comprises the amino acid sequence VIWYDGSKRYYADSVKG (SEQ ID NO:42), the VH CDR3 comprises the amino acid sequence NDDY (SEQ ID NO:43), and which has at least 75%, 80%, 85%, 90%, 95%, 96%, 97%, 98%, 99%, or 100% identity to the amino acid sequence of SEQ ID NO:47, the VL CDR1 comprises the amino acid sequence RASQSVSSYLA (SEQ ID NO:44), the VL CDR2 comprises the amino acid sequence DASNRAT (SEQ ID NO:45), and the VL CDR3 comprises the amino acid sequence QQSSNWPRT (SEQ ID NO:46), and which has at least 75%, 80%, 85%, 90%, 95%, 96%, 97%, 98%, 99%, or 100% identity to the amino acid sequence of SEQ ID NO:48.

In some embodiments, the anti-PD-1 antibody comprises a heavy chain variable region with one or more (e.g., 1, 2, or 3) substitutions, deletions, or insertions in the amino acid sequence of SEQ ID NO:47, and a light chain variable region with one or more (e.g., 1, 2, or 3) substitutions, deletions, or insertions in the amino acid sequence of SEQ ID NO:48.

In some embodiments, the anti-PD-1 antibody comprises a heavy chain variable region comprising the amino acid sequence of SEQ ID NO:47 and a light chain variable region comprising the amino acid sequence of SEQ ID NO:48.

In some embodiments, the anti-PD-1 antibody comprises a heavy chain that is at least 75%, 80%, 85%, 90%, 95%, 96%, 97%, 98%, 99%, or 100% identical to the amino acid sequence of SEQ ID NO:49 and a light chain that is at least 75%, 80%, 85%, 90%, 95%, 96%, 97%, 98%, 99%, or 100% identical to the amino acid sequence of SEQ ID NO:50.

In some embodiments, the anti-PD-1 antibody comprises a heavy chain comprising the amino acid sequence of SEQ ID NO:49 and a light chain comprising the amino acid sequence of SEQ ID NO:50.

In some embodiments, the anti-PD-1 antibody comprises retifanlimab. In some embodiments, the anti-PD-1 antibody comprises pembrolizumab. In some embodiments, the anti-PD-1 antibody comprises nivolumab. In some embodiments, the anti-PD-1 antibody comprises spartalizumab.

Non-limiting examples of anti-PD-1 antibodies that can be used in methods described herein are provided in U.S. Pat. No. 10,577,422 and Publication No. US 2020/0095322 A1, each of which is incorporated herein by reference in its entirety. The amino acid sequences of exemplary anti-PD-1 antibodies, retifanlimab, pembrolizumab, nivolumab, and spartalizumab, are described in Table 2.

TABLE 2
Anti-PD-1 Antibody Sequences
Retifanlimab

| Antibody | |
|---|---|
| Feature | Sequences |
| VH CDR1 | SYWMN (SEQ ID NO: 11) |
| VH CDR2 | VIHPSDSETWLDQKFKD (SEQ ID NO: 12) |
| VH CDR3 | EHYGTSPFAY (SEQ ID NO: 13) |
| VL CDR1 | RASESVDNYGMSFMNW (SEQ ID NO: 14) |
| VL CDR2 | AASNQGS (SEQ ID NO: 15) |
| VL CDR3 | QQSKEVPYT (SEQ ID NO: 16) |
| VH | QVQLVQSGAEVKKPGASVKVSCKASGYSFTSYWMNWVRQAPGQGLEWIGVIHPSDSE |
| | TWLDQKFKDRVTITVDKSTSTAYMELSSLRSEDTAVYYCAREHYGTSPFAYWGQGTL |
| | VTVSS (SEQ ID NO: 17) |
| VL | EIVLTQSPATLSLSPGERATLSCRASESVDNYGMSFMNWFQQKPGQPPKLLIHAASN |
| | QGSGVPSRFSGSGSGTDFTLTISSLEPEDFAVYFCQQSKEVPYTFGGGTKVEIK |
| | (SEQ ID NO: 18) |
| HC | QVQLVQSGAEVKKPGASVKVSCKASGYSFTSYWMNWVRQAPGQGLEWIGVIHPSDSE |
| | TWLDQKFKDRVTITVDKSTSTAYMELSSLRSEDTAVYYCAREHYGTSPFAYWGQGTL |
| | VTVSSASTKGPSVFPLAPCSRSTSESTAALGCLVKDYFPEPVTVSWNSGALTSGVHT |
| | FPAVLQSSGLYSLSSVVTVPSSSLGTKTYTCNVDHKPSNTKVDKRVESKYGPPCPPC |
| | PAPEFLGGPSVFLFPPKPKDTLMISRTPEVTCVVVDVSQEDPEVQFNWYVDGVEVHN |
| | AKTKPREEQFNSTYRVVSVLTVLHQDWLNGKEYKCKVSNKGLPSSIEKTISKAKGQP |
| | REPQVYTLPPSQEEMTKNQVSLTCLVKGFYPSDIAVEWESNGQPENNYKTTPPVLDS |
| | DGSFFLYSRLTVDKSRWQEGNVFSCSVMHEALHNHYTQKSLSLSLG (SEQ ID |
| | NO: 19) |
| LC | EIVLTQSPATLSLSPGERATLSCRASESVDNYGMSFMNWFQQKPGQPPKLLIHAASN |
| | QGSGVPSRFSGSGSGTDFTLTISSLEPEDFAVYFCQQSKEVPYTFGGGTKVEIKRTV |
| | AAPSVFIFPPSDEQLKSGTASVVCLLNNFYPREAKVQWKVDNALQSGNSQESVTEQD |
| | SKDSTYSLSSTLTLSKADYEKHKVYACEVTHQGLSSPVTKSFNRGEC (SEQ ID |
| | NO: 20) |

Pembrolizumab

| Antibody | |
|---|---|
| Feature | Sequences |
| VH CDR1 | NYYMY (SEQ ID NO: 21) |
| VH CDR2 | GINPSNGGTNFNEKFKN (SEQ ID NO: 22) |
| VH CDR3 | RDYRFDMGFDY (SEQ ID NO: 23) |
| VL CDR1 | RASKGVSTSGYSYLH (SEQ ID NO: 24) |
| VL CDR2 | LASYLES (SEQ ID NO: 25) |
| VL CDR3 | QHSRDLPLT (SEQ ID NO: 26) |
| VH | QVQLVQSGVEVKKPGASVKVSCKASGYTFTNYYMYWVRQAPGQGLEWMGGINPSNGG |
| | TNFNEKFKNRVTLTTDSSTTTAYMELKSLQFDDTAVYYCARRDYRFDMGFDYWGQGT |
| | TVTVSS (SEQ ID NO: 27) |
| VL | EIVLTQSPATLSLSPGERATLSCRASKGVSTSGYSYLHWYQQKPGQAPRLLIYLASY |
| | LESGVPARFSGSGSGTDFTLTISSLEPEDFAVYYCQHSRDLPLTFGGGTKVEIK |
| | (SEQ ID NO: 28) |
| HC | QVQLVQSGVEVKKPGASVKVSCKASGYTFTNYYMYWVRQAPGQGLEWMGGINPSNGG |
| | TNFNEKFKNRVTLTTDSSTTTAYMELKSLQFDDTAVYYCARRDYRFDMGFDYWGQGT |
| | TVTVSSASTKGPSVFPLAPCSRSTSESTAALGCLVKDYFPEPVTVSWNSGALTSGVH |
| | TFPAVLQSSGLYSLSSVVTVPSSSLGTKTYTCNVDHKPSNTKVDKRVESKYGPPCPP |
| | CPAPEFLGGPSVELFPPKPKDTLMISRTPEVTCVVVDVSQEDPEVQFNWYVDGVEVH |
| | NAKTKPREEQFNSTYRVVSVLTVLHQDWLNGKEYKCKVSNKGLPSSIEKTISKAKGQ |
| | PREPQVYTLPPSQEEMTKNQVSLTCLVKGFYPSDIAVEWESNGQPENNYKTTPPVLD |
| | SDGSFFLYSRLTVDKSRWQEGNVFSCSVMHEALHNHYTQKSLSLSLGK (SEQ ID |
| | NO: 29) |
| LC | EIVLTQSPATLSLSPGERATLSCRASKGVSTSGYSYLHWYQQKPGQAPRLLIYLASY |
| | LESGVPARFSGSGSGTDFTLTISSLEPEDFAVYYCQHSRDLPLTFGGGTKVEIKRTV |
| | AAPSVFIFPPSDEQLKSGTASVVCLLNNFYPREAKVQWKVDNALQSGNSQESVTEQD |
| | SKDSTYSLSSTLTLSKADYEKHKVYACEVTHQGLSSPVTKSFNRGEC (SEQ ID |
| | NO: 30) |

Nivolumab

| Antibody | |
|---|---|
| Feature | Sequences |
| VH CDR1 | NSGMH (SEQ ID NO: 31) |
| VH CDR2 | VIWYDGSKRYYADSVKG (SEQ ID NO: 32) |
| VH CDR3 | NDDY (SEQ ID NO: 33) |
| VL CDR1 | RASQSVSSYLA (SEQ ID NO: 34) |
| VL CDR2 | DASNRAT (SEQ ID NO: 35) |
| VL CDR3 | QQSSNWPRT (SEQ ID NO: 36) |
| VH | QVQLVESGGGVVQPGRSLRLDCKASGITFSNSGMHWVRQAPGKGLEWVAVIWYDGSK |
| | RYYADSVKGRFTISRDNSKNTLFLQMNSLRAEDTAVYYCATNDDYWGQGTLVTVSS |
| | (SEQ ID NO: 37) |
| VL | EIVLTQSPATLSLSPGERATLSCRASQSVSSYLAWYQQKPGQAPRLLIYDASNRATG |
| | IPARFSGSGSGTDFTLTISSLEPEDFAVYYCQQSSNWPRTFGQGTKVEIK (SEQ |
| | ID NO: 38) |
| HC | QVQLVESGGGVVQPGRSLRLDCKASGITFSNSGMHWVRQAPGKGLEWVAVIWYDGSK |
| | RYYADSVKGRFTISRDNSKNTLFLQMNSLRAEDTAVYYCATNDDYWGQGTLVTVSSA |
| | STKGPSVFPLAPCSRSTSESTAALGCLVKDYFPEPVTVSWNSGALTSGVHTFPAVLQ |
| | SSGLYSLSSVVTVPSSSLGTKTYTCNVDHKPSNTKVDKRVESKYGPPCPPCPAPEFL |
| | GGPSVFLFPPKPKDTLMISRTPEVTCVVVDVSQEDPEVQFNWYVDGVEVHNAKTKPR |
| | EEQFNSTYRVVSVLTVLHQDWLNGKEYKCKVSNKGLPSSIEKTISKAKGQPREPQVY |
| | TLPPSQEEMTKNQVSLTCLVKGFYPSDIAVEWESNGQPENNYKTTPPVLDSDGSFEL |
| | YSRLTVDKSRWQEGNVFSCSVMHEALHNHYTQKSLSLSLGK (SEQ ID NO: 39) |
| LC | EIVLTQSPATLSLSPGERATLSCRASQSVSSYLAWYQQKPGQAPRLLIYDASNRATG |
| | IPARFSGSGSGTDFTLTISSLEPEDFAVYYCQQSSNWPRTFGQGTKVEIKRTVAAPS |
| | VFIFPPSDEQLKSGTASVVCLLNNFYPREAKVQWKVDNALQSGNSQESVTEQDSKDS |
| | TYSLSSTLTLSKADYEKHKVYACEVTHQGLSSPVTKSFNRGEC (SEQ ID NO: |
| | 40) |

Spartalizumab

| Antibody | |
|---|---|
| Feature | Sequences |
| VH CDR1 | TYWMH (SEQ ID NO: 107) |
| VH CDR2 | NIYPGTGGSNFDEKFKN (SEQ ID NO: 108) |
| VH CDR3 | WTTGTGAY (SEQ ID NO: 109) |
| VL CDR1 | KSSQSLLDSGNQKNFLT (SEQ ID NO: 110) |
| VL CDR2 | WASTRES (SEQ ID NO: 5) |
| VL CDR3 | QNDYSYPYT (SEQ ID NO: 6) |
| VH | EVQLVQSGAEVKKPGESLRISCKGSGYTFTTYWMHWVRQATGQGLEWMGNIYPGTGG |
| | SNFDEKFKNRVTITADKSTSTAYMELSSLRSEDTAVYYCTRWTTGTGAYWGQGTTVT |
| | VSS (SEQ ID NO: 47) |
| VL | EIVLTQSPATLSLSPGERATLSCKSSQSLLDSGNQKNFLTWYQQKPGQAPRLLIYWA |
| | STRESGVPSRFSGSGSGTDFTFTISSLEAEDAATYYCQNDYSYPYTFGQGTKVEIK |
| | (SEQ ID NO: 48) |
| HC | EVQLVQSGAEVKKPGESLRISCKGSGYTFTTYWMHWVRQATGQGLEWMGNIYPGTGG |
| | SNFDEKFKNRVTITADKSTSTAYMELSSLRSEDTAVYYCTRWTTGTGAYWGQGTTVT |
| | VSSASTKGPSVFPLAPCSRSTSESTAALGCLVKDYFPEPVTVSWNSGALTSGVHTFP |
| | AVLQSSGLYSLSSVVTVPSSSLGTKTYTCNVDHKPSNTKVDKRVESKYGPPCPPCPA |
| | PEFLGGPSVFLFPPKPKDTLMISRTPEVTCVVVDVSQEDPEVQFNWYVDGVEVHNAK |
| | TKPREEQFNSTYRVVSVLTVLHQDWLNGKEYKCKVSNKGLPSSIEKTISKAKGQPRE |
| | PQVYTLPPSQEEMTKNQVSLTCLVKGFYPSDIAVEWESNGQPENNYKTTPPVLDSDG |
| | SFFLYSRLTVDKSRWQEGNVFSCSVMHEALHNHYTQKSLSLSLG (SEQ ID |
| | NO: 49) |
| LC | EIVLTQSPATLSLSPGERATLSCKSSQSLLDSGNQKNFLTWYQQKPGQAPRLLIYWA |
| | STRESGVPSRFSGSGSGTDFTFTISSLEAEDAATYYCQNDYSYPYTFGQGTKVEIKR |
| | TVAAPSVFIFPPSDEQLKSGTASVVCLLNNFYPREAKVQWKVDNALQSGNSQESVTE |
| | QDSKDSTYSLSSTLTLSKADYEKHKVYACEVTHQGLSSPVTKSFNRGEC (SEQ ID |
| | NO: 50) |

Non-limiting examples of a PD-L1 inhibitor for use in the methods described herein include a small molecule, anti-PD-L1 antibody, or a peptide that inhibits PD-L1 (e.g., a peptide aptamer, a PD-L1 structural analog).

In some examples, a PD-L1 inhibitor comprises an anti-PD-L1 antibody.

In some embodiments, the anti-PD-L1 antibody comprises a VH comprising a VH CDR1, a VH CDR2, and a VH CDR3, and a VL comprising a VL CDR1, a VL CDR2, and a VL CDR3, wherein the VH CDR1 comprises the amino acid sequence DSWIH (SEQ ID NO:51), the VH CDR2 comprises the amino acid sequence WISPYGGSTY (SEQ ID NO:52), the VH CDR3 comprises the amino acid sequence RHWPGGF (SEQ ID NO:53), the VL CDR1 comprises the amino acid sequence DVSTAVA (SEQ ID NO:54), the VL CDR2 comprises the amino acid sequence SASFLY (SEQ ID NO:55), and the VL CDR3 comprises the amino acid sequence QQYLYHPAT (SEQ ID NO:56).

In some embodiments, the anti-PD-L1 antibody comprises a VH comprising a VH CDR1, a VH CDR2, and a VH CDR3, and a VL comprising a VL CDR1, a VL CDR2, and a VL CDR3, wherein the VH CDR1 comprises the amino acid sequence DSWIH (SEQ ID NO:51), or a variant thereof comprising 1, 2, 3, or 4 amino acid substitutions; the VH CDR2 comprises the amino acid sequence WISPYGGSTY (SEQ ID NO:52), or a variant thereof comprising 1, 2, 3, or 4 amino acid substitutions; the VH CDR3 comprises the amino acid sequence RHWPGGF (SEQ ID NO:53), or a variant thereof comprising 1, 2, 3, or 4 amino acid substitutions; the VL CDR1 comprises the amino acid sequence DVSTAVA (SEQ ID NO:54), or a variant thereof comprising 1, 2, 3, or 4 amino acid substitutions; the VL CDR2 comprises the amino acid sequence SASFLY (SEQ ID NO:55), or a variant thereof comprising 1, 2, 3, or 4 amino acid substitutions; and the VL CDR3 comprises the amino acid sequence QQYLYHPAT (SEQ ID NO:56), or a variant thereof comprising 1, 2, 3, or 4 amino acid substitutions.

In some embodiments, the anti-PD-L1 antibody comprises a VH comprising a VH CDR1, a VH CDR2, and a VH CDR3, and a VL comprising a VL CDR1, a VL CDR2, and a VL CDR3, wherein the VH CDR1 comprises the amino acid sequence DSWIH (SEQ ID NO:51), the VH CDR2 comprises the amino acid sequence WISPYGGSTY (SEQ ID NO:52), the VH CDR3 comprises the amino acid sequence RHWPGGF (SEQ ID NO:53), and which has at least 75%, 80%, 85%, 90%, 95%, 96%, 97%, 98%, 99%, or 100% identity to the amino acid sequence of SEQ ID NO:57, the VL CDR1 comprises the amino acid sequence DVSTAVA (SEQ ID NO:54), the VL CDR2 comprises the amino acid sequence SASFLY (SEQ ID NO:55), and the VL CDR3 comprises the amino acid sequence QQYLYHPAT (SEQ ID NO:56), and which has at least 75%, 80%, 85%, 90%, 95%, 96%, 97%, 98%, 99%, or 100% identity to the amino acid sequence of SEQ ID NO:58.

In some embodiments, the anti-PD-1 antibody comprises a heavy chain variable region with one or more (e.g., 1, 2, or 3) substitutions, deletions, or insertions in the amino acid sequence of SEQ ID NO:57, and a light chain variable region with one or more (e.g., 1, 2, or 3) substitutions, deletions, or insertions in the amino acid sequence of SEQ ID NO:58.

In some embodiments, the anti-PD-1 antibody comprises a heavy chain variable region comprising the amino acid sequence of SEQ ID NO:57 and a light chain variable region comprising the amino acid sequence of SEQ ID NO:58.

In some embodiments, the anti-PD-1 antibody comprises a heavy chain that is at least 75%, 80%, 85%, 90%, 95%, 96%, 97%, 98%, 99%, or 100% identical to the amino acid sequence of SEQ ID NO:59 and a light chain that is at least 75%, 80%, 85%, 90%, 95%, 96%, 97%, 98%, 99%, or 100% identical to the amino acid sequence of SEQ ID NO:60.

In some embodiments, the anti-PD-1 antibody comprises a heavy chain comprising the amino acid sequence of SEQ ID NO:59 and a light chain comprising the amino acid sequence of SEQ ID NO:60.

In some embodiments, the anti-PD-L1 antibody comprises a VH comprising a VH CDR1, a VH CDR2, and a VH CDR3, and a VL comprising a VL CDR1, a VL CDR2, and a VL CDR3, wherein the VH CDR1 comprises the amino acid sequence SYIMM (SEQ ID NO:61), the VH CDR2 comprises the amino acid sequence SIYPSGGITF (SEQ ID NO:62), the VH CDR3 comprises the amino acid sequence IKLGTVTTV (SEQ ID NO:63), the VL CDR1 comprises the amino acid sequence VGGYNYVS (SEQ ID NO:64), the VL CDR2 comprises the amino acid sequence DVSNRP (SEQ ID NO:65), and the VL CDR3 comprises the amino acid sequence SSYTSSSTRV (SEQ ID NO:66).

In some embodiments, the anti-PD-L1 antibody comprises a VH comprising a VH CDR1, a VH CDR2, and a VH CDR3, and a VL comprising a VL CDR1, a VL CDR2, and a VL CDR3, wherein the VH CDR1 comprises the amino acid sequence SYIMM (SEQ ID NO:61), or a variant thereof comprising 1, 2, 3, or 4 amino acid substitutions; the VH CDR2 comprises the amino acid sequence SIYPSGGITF (SEQ ID NO:62), or a variant thereof comprising 1, 2, 3, or 4 amino acid substitutions; the VH CDR3 comprises the amino acid sequence IKLGTVTTV (SEQ ID NO:63), or a variant thereof comprising 1, 2, 3, or 4 amino acid substitutions; the VL CDR1 comprises the amino acid sequence VGGYNYVS (SEQ ID NO:64), or a variant thereof comprising 1, 2, 3, or 4 amino acid substitutions; the VL CDR2 comprises the amino acid sequence DVSNRP (SEQ ID NO:65), or a variant thereof comprising 1, 2, 3, or 4 amino acid substitutions; and the VL CDR3 comprises the amino acid sequence SSYTSSSTRV (SEQ ID NO:66), or a variant thereof comprising 1, 2, 3, or 4 amino acid substitutions.

In some embodiments, the anti-PD-L1 antibody comprises a VH comprising a VH CDR1, a VH CDR2, and a VH CDR3, and a VL comprising a VL CDR1, a VL CDR2, and a VL CDR3, wherein the VH CDR1 comprises the amino acid sequence SYIMM (SEQ ID NO:61), the VH CDR2 comprises the amino acid sequence SIYPSGGITF (SEQ ID NO:62), the VH CDR3 comprises the amino acid sequence IKLGTVTTV (SEQ ID NO:63), and which has at least 75%, 80%, 85%, 90%, 95%, 96%, 97%, 98%, 99%, or 100% identity to the amino acid sequence of SEQ ID NO:67, the VL CDR1 comprises the amino acid sequence VGGYNYVS (SEQ ID NO:64), the VL CDR2 comprises the amino acid sequence DVSNRP (SEQ ID NO:65), and the VL CDR3 comprises the amino acid sequence SSYTSSSTRV (SEQ ID NO:66), and which has at least 75%, 80%, 85%, 90%, 95%, 96%, 97%, 98%, 99%, or 100% identity to the amino acid sequence of SEQ ID NO:68.

In some embodiments, the anti-PD-1 antibody comprises a heavy chain variable region with one or more (e.g., 1, 2, or 3) substitutions, deletions, or insertions in the amino acid sequence of SEQ ID NO:67, and a light chain variable region with one or more (e.g., 1, 2, or 3) substitutions, deletions, or insertions in the amino acid sequence of SEQ ID NO:68.

In some embodiments, the anti-PD-1 antibody comprises a heavy chain variable region comprising the amino acid sequence of SEQ ID NO:67 and a light chain variable region comprising the amino acid sequence of SEQ ID NO:68.

In some embodiments, the anti-PD-1 antibody comprises a heavy chain that is at least 75%, 80%, 85%, 90%, 95%, 96%, 97%, 98%, 99%, or 100% identical to the amino acid sequence of SEQ ID NO:69 and a light chain that is at least 75%, 80%, 85%, 90%, 95%, 96%, 97%, 98%, 99%, or 100% identical to the amino acid sequence of SEQ ID NO:70.

In some embodiments, the anti-PD-1 antibody comprises a heavy chain comprising the amino acid sequence of SEQ ID NO:69 and a light chain comprising the amino acid sequence of SEQ ID NO:70.

In some embodiments, the anti-PD-L1 antibody comprises a VH comprising a VH CDR1, a VH CDR2, and a VH CDR3, and a VL comprising a VL CDR1, a VL CDR2, and a VL CDR3, wherein the VH CDR1 comprises the amino acid sequence RYWMS (SEQ ID NO:71), the VH CDR2 comprises the amino acid sequence NIKQDGSEKY (SEQ ID NO:72), the VH CDR3 comprises the amino acid sequence EGGWFGELAF (SEQ ID NO:73), the VL CDR1 comprises the amino acid sequence RVSSSYLA (SEQ ID NO:74), the VL CDR2 comprises the amino acid sequence DASSRA (SEQ ID NO:75), and the VL CDR3 comprises the amino acid sequence QQYGSLPWT (SEQ ID NO:76).

In some embodiments, the anti-PD-L1 antibody comprises a VH comprising a VH CDR1, a VH CDR2, and a VH CDR3, and a VL comprising a VL CDR1, a VL CDR2, and a VL CDR3, wherein the VH CDR1 comprises the amino acid sequence RYWMS (SEQ ID NO:71), or a variant thereof comprising 1, 2, 3, or 4 amino acid substitutions; the VH CDR2 comprises the amino acid sequence NIKQDGSEKY (SEQ ID NO:72), or a variant thereof comprising 1, 2, 3, or 4 amino acid substitutions; the VH CDR3 comprises the amino acid sequence EGGWFGELAF (SEQ ID NO:73), or a variant thereof comprising 1, 2, 3, or 4 amino acid substitutions; the VL CDR1 comprises the amino acid sequence RVSSSYLA (SEQ ID NO:74), or a variant thereof comprising 1, 2, 3, or 4 amino acid substitutions; the VL CDR2 comprises the amino acid sequence DASSRA (SEQ ID NO:75), or a variant thereof comprising 1, 2, 3, or 4 amino acid substitutions; and the VL CDR3 comprises the amino acid sequence QQYGSLPWT (SEQ ID NO:76), or a variant thereof comprising 1, 2, 3, or 4 amino acid substitutions.

In some embodiments, the anti-PD-L1 antibody comprises a VH comprising a VH CDR1, a VH CDR2, and a VH CDR3, and a VL comprising a VL CDR1, a VL CDR2, and a VL CDR3, wherein the VH CDR1 comprises the amino acid sequence RYWMS (SEQ ID NO:71), the VH CDR2 comprises the amino acid sequence NIKQDGSEKY (SEQ ID NO:72), the VH CDR3 comprises the amino acid sequence EGGWFGELAF (SEQ ID NO:73), and which has at least 75%, 80%, 85%, 90%, 95%, 96%, 97%, 98%, 99%, or 100% identity to the amino acid sequence of SEQ ID NO:77, the VL CDR1 comprises the amino acid sequence RVSSSYLA (SEQ ID NO:74), the VL CDR2 comprises the amino acid sequence DASSRA (SEQ ID NO:75), and the VL CDR3 comprises the amino acid sequence QQYGSLPWT (SEQ ID NO:76), and which has at least 75%, 80%, 85%, 90%, 95%, 96%, 97%, 98%, 99%, or 100% identity to the amino acid sequence of SEQ ID NO:78.

In some embodiments, the anti-PD-1 antibody comprises a heavy chain variable region with one or more (e.g., 1, 2, or 3) substitutions, deletions, or insertions in the amino acid sequence of SEQ ID NO:77, and a light chain variable region with one or more (e.g., 1, 2, or 3) substitutions, deletions, or insertions in the amino acid sequence of SEQ ID NO:78.

In some embodiments, the anti-PD-1 antibody comprises a heavy chain variable region comprising the amino acid sequence of SEQ ID NO:77 and a light chain variable region comprising the amino acid sequence of SEQ ID NO:78.

In some embodiments, the anti-PD-1 antibody comprises a heavy chain that is at least 75%, 80%, 85%, 90%, 95%, 96%, 97%, 98%, 99%, or 100% identical to the amino acid sequence of SEQ ID NO:79 and a light chain that is at least 75%, 80%, 85%, 90%, 95%, 96%, 97%, 98%, 99%, or 100% identical to the amino acid sequence of SEQ ID NO:80.

In some embodiments, the anti-PD-1 antibody comprises a heavy chain comprising the amino acid sequence of SEQ ID NO:79 and a light chain comprising the amino acid sequence of SEQ ID NO:80.

In some embodiments, the anti-PD-L1 antibody comprises atezolizumab. In some embodiments, the anti-PD-L1 antibody comprises avelumab. In some embodiments, the anti-PD-L1 antibody comprises durvalumab.

Non-limiting examples of anti-PD-L1 antibodies that can be used in methods described herein are provided in U.S. Pat. Nos. 8,217,149 and 8,779,108 Publication No. US 2014/341917 A1, each of which is incorporated herein by reference in its entirety. The amino acid sequences of exemplary anti-PD-L1 antibodies, atezolizumab, avelumab, and durvalumab, are described in Table 3.

TABLE 3
Anti-PD-L1 Antibody Sequences
Atezolizumab

| Antibody | |
|---|---|
| Feature | Sequences |
| VH CDR1 | DSWIH (SEQ ID NO: 51) |
| VH CDR2 | WISPYGGSTY (SEQ ID NO: 52) |
| VH CDR3 | RHWPGGF (SEQ ID NO: 53) |
| VL CDR1 | DVSTAVA (SEQ ID NO: 54) |
| VL CDR2 | SASFLY (SEQ ID NO: 55) |
| VL CDR3 | QQYLYHPAT (SEQ ID NO: 56) |
| VH | EVQLVESGGGLVQPGGSLRLSCAASGFTFSDSWIHWVRQAPGKGLEWVAWI |
| | SPYGGSTYYADSVKGRFTISADTSKNTAYLQMNSLRAEDTAVYYCARRHWP |
| | GGFDYWGQGTLVT (SEQ ID NO: 57) |
| VL | DIQMTQSPSSLSASVGDRVTITCRASQDVSTAVAWYQQKPGKAPKLLIYSA |
| | SFLYSGVPSRFSGSGSGTDFTLTISSLQPEDFATYYCQQYLYHPATFGQGT |
| | KVEIK (SEQ ID NO: 58) |
| HC | EVQLVESGGGLVQPGGSLRLSCAASGFTFSDSWIHWVRQAPGKGLEWVAWISPYGG |
| | STYYADSVKGRFTISADTSKNTAYLQMNSLRAEDTAVYYCARRHWPGGFDYWGQGT |
| | LVTVSSASTKGPSVFPLAPSSKSTSGGTAALGCLVKDYFPEPVTVSWNSGALTSGV |
| | HTFPAVLQSSGLYSLSSVVTVPSSSLGTQTYICNVNHKPSNTKVDKKVEPKSCDKT |
| | HTCPPCPAPELLGGPSVFLFPPKPKDTLMISRTPEVTCVVVDVSHEDPEVKFNWYV |
| | DGVEVHNAKTKPREEQYASTYRVVSVLTVLHQDWLNGKEYKCKVSNKALPAPIEKT |
| | ISKAKGQPREPQVYTLPPSREEMTKNQVSLTCLVKGFYPSDIAVEWESNGQPENNY |
| | KTTPPVLDSDGSFFLYSKLTVDKSRWQQGNVFSCSVMHEALHNHYTQKSLSLSPGK |
| | (SEQ ID NO: 59) |
| LC | DIQMTQSPSSLSASVGDRVTITCRASQDVSTAVAWYQQKPGKAPKLLIYSASFLYS |
| | GVPSRFSGSGSGTDFTLTISSLQPEDFATYYCQQYLYHPATFGQGTKVEIKRTVAA |
| | PSVFIFPPSDEQLKSGTASVVCLLNNFYPREAKVQWKVDNALQSGNSQESVTEQDS |
| | KDSTYSLSSTLTLSKADYEKHKVYACEVTHQGLSSPVTKSFNRGEC (SEQ ID |
| | NO: 60) |

Avelumab

| Antibody | |
|---|---|
| Feature | Sequences |
| VH CDR1 | SYIMM (SEQ ID NO: 61) |
| VH CDR2 | SIYPSGGITF (SEQ ID NO: 62) |
| VH CDR3 | IKLGTVTTV (SEQ ID NO: 63) |
| VL CDR1 | VGGYNYVS (SEQ ID NO: 64) |
| VL CDR2 | DVSNRP (SEQ ID NO: 65) |
| VL CDR3 | SSYTSSSTRV (SEQ ID NO: 66) |
| VH | EVQLLESGGGLVQPGGSLRLSCAASGFTFSSYIMMWVRQAPGKGLEWVSSI |
| | YPSGGITFYADTVKGRFTISRDNSKNTLYLQMNSLRAEDTAVYYCARIKLG |
| | TVTTVDYWGQGTLVT (SEQ ID NO: 67) |
| VL | QSALTQPASVSGSPGQSITISCTGTSSDVGGYNYVSWYQQHPGKAPKLMIY |
| | DVSNRPSGVSNRFSGSKSGNTASLTISGLQAEDEADYYCSSYTSSSTRVFG |
| | TGTKVTVL (SEQ ID NO: 68) |
| HC | EVQLLESGGGLVQPGGSLRLSCAASGFTFSSYIMMWVRQAPGKGLEWVSSIYPSGG |
| | ITFYADTVKGRFTISRDNSKNTLYLQMNSLRAEDTAVYYCARIKLGTVTTVDYWGQ |
| | GTLVTVSSASTKGPSVFPLAPSSKSTSGGTAALGCLVKDYFPEPVTVSWNSGALTS |
| | GVHTFPAVLQSSGLYSLSSVVTVPSSSLGTQTYICNVNHKPSNTKVDKKVEPKSCD |
| | KTHTCPPCPAPELLGGPSVFLFPPKPKDTLMISRTPEVTCVVVDVSHEDPEVKFNW |
| | YVDGVEVHNAKTKPREEQYNSTYRVVSVLTVLHQDWLNGKEYKCKVSNKALPAPIE |
| | KTISKAKGQPREPQVYTLPPSRDELTKNQVSLTCLVKGFYPSDIAVEWESNGQPEN |
| | NYKTTPPVLDSDGSFFLYSKLTVDKSRWQQGNVFSCSVMHEALHNHYTQKSLSLSP |
| | GK (SEQ ID NO: 69) |
| LC | QSALTQPASVSGSPGQSITISCTGTSSDVGGYNYVSWYQQHPGKAPKLMIYDVSNR |
| | PSGVSNRFSGSKSGNTASLTISGLQAEDEADYYCSSYTSSSTRVFGTGTKVTVLGQ |
| | PKANPTVTLFPPSSEELQANKATLVCLISDFYPGAVTVAWKADGSPVKAGVETTKP |
| | SKQSNNKYAASSYLSLTPEQWKSHRSYSCQVTHEGSTVEKTVAPTECS (SEQ ID |
| | NO: 70) |

Durvalumab

| Antibody | |
|---|---|
| Feature | Sequences |
| VH CDR1 | RYWMS (SEQ ID NO: 71) |
| VH CDR2 | NIKQDGSEKY (SEQ ID NO: 72) |
| VH CDR3 | EGGWFGELAF (SEQ ID NO: 73) |
| VL CDR1 | RVSSSYLA (SEQ ID NO: 74) |
| VL CDR2 | DASSRA (SEQ ID NO: 75) |
| VL CDR3 | QQYGSLPWT (SEQ ID NO: 76) |
| VH | EVQLVESGGGLVQPGGSLRLSCAASGFTFSRYWMSWVRQAPGKGLEWVANI |
| | KQDGSEKYYVDSVKGRFTISRDNAKNSLYLQMNSLRAEDTAVYYCAREGGW |
| | FGELAFDYWGQGTLVT (SEQ ID NO: 77) |
| VL | EIVLTQSPGTLSLSPGERATLSCRASQRVSSSYLAWYQQKPGQAPRLLIYD |
| | ASSRATGIPDRFSGSGSGTDFTLTISRLEPEDFAVYYCQQYGSLPWTFGQG |
| | TKVEIK (SEQ ID NO: 78) |
| HC | EVQLVESGGGLVQPGGSLRLSCAASGFTFSRYWMSWVRQAPGKGLEWVANI |
| | KQDGSEKYYVDSVKGRFTISRDNAKNSLYLQMNSLRAEDTAVYYCAREGGW |
| | FGELAFDYWGQGTLVTVSSASTKGPSVFPLAPSSKSTSGGTAALGCLVKDY |
| | FPEPVTVSWNSGALTSGVHTFPAVLQSSGLYSLSSVVTVPSSSLGTQTYIC |
| | NVNHKPSNTKVDKRVEPKSCDKTHTCPPCPAPEFEGGPSVFLFPPKPKDTL |
| | MISRTPEVTCVVVDVSHEDPEVKFNWYVDGVEVHNAKTKPREEQYNSTYRV |
| | VSVLTVLHQDWLNGKEYKCKVSNKALPASIEKTISKAKGQPREPQVYTLPP |
| | SREEMTKNQVSLTCLVKGFYPSDIAVEWESNGQPENNYKTTPPVLDSDGSF |
| | FLYSKLTVDKSRWQQGNVFSCSVMHEALHNHYTQKSLSLSPGK (SEQ ID |
| | NO: 79) |
| LC | EIVLTQSPGTLSLSPGERATLSCRASQRVSSSYLAWYQQKPGQAPRLLIYD |
| | ASSRATGIPDRESGSGSGTDFTLTISRLEPEDFAVYYCQQYGSLPWTFGQG |
| | TKVEIKRTVAAPSVFIFPPSDEQLKSGTASVVCLLNNFYPREAKVQWKVDN |
| | ALQSGNSQESVTEQDSKDSTYSLSSTLTLSKADYEKHKVYACEVTHQGLSS |
| | PVTKSFNRGEC (SEQ ID NO: 80) |

### Inhibitors of CTLA-4

Cytotoxic T-lymphocyte-associated protein 4 (CTLA-4) (also known as CD152) is an immune inhibitory receptor constitutively expressed on Tregs and upregulated in activated T cells. CTLA-4 plays a critical role in regulating immune responses to tumor antigens and autoantigens. When CTLA-4 expression is upregulated on the surface of T cells, the T cells bind B7 with a higher avidity, and thus outcompete the positive co-stimulatory signal from CD28. Engagement of CTLA-4 by either of its ligands, CD80 (B7-1) or CD86 (B7-2) on an adjacent antigen presenting cell (APC) inhibits CD28 co-stimulation of T cell activation, cell proliferation and cytokine production.

The amino acid sequence of the mature human CTLA-4 protein (amino acids 1-223) of NP_005205.2 is:

(SEQ ID NO: 83)
MACLGFQRHKAQLNLATRTWPCTLLFFLLFIPVFCKAMHVAQPAVVLAS
SRGIASFVCEYASPGKATEVRVTVLRQADSQVTEVCAATYMMGNELTEL
DDSICTGTSSGNQVNLTIQGLRAMDTGLYICKVELMYPPPYYLGIGNGT
QIYVIDPEPCPDSDELLWILAAVSSGLFFYSELLTAVSLSKMLKKRSPL
TTGVYVKMPPTEPECEKQFQPYFIPIN

This disclosure provides CTLA-4 inhibitors (e.g., anti-CTLA-4 antibodies) that are useful in treating cancer.

The term “CTLA-4 inhibitor” as used herein, refers to a molecule that binds to CTLA-4 (e.g., a small molecule or a biological molecule) and blocks, inhibits, reduces (including significantly), or interferes with CTLA-4 biological activity.

In some embodiments, a CTLA-4 inhibitor is a molecule that inhibits or disrupts CTLA-4 itself (e.g., human CTLA-4), a biological activity of CTLA-4 (e.g., including but not limited to its ability to mediate any aspect of cancer), or the consequences of the biological activity to any meaningful degree, e.g., at least 10%, at least 20%, at least 30%, at least 40%, at least 50%, at least 60%, at least 70%, at least 80%, at least 90%, or more.

Non-limiting examples of a CTLA-4 inhibitor for use in the methods described herein include a small molecule, anti-CTLA-4 antibody, or a peptide that inhibits CTLA-4 (e.g., a peptide aptamer or a CTLA-4 structural analog).

In some examples, the CTLA-4 inhibitor comprises an anti-CTLA-4 antibody.

In some embodiments, the anti-CTLA-4 antibody comprises a heavy chain variable region comprising a VH CDR1 comprising the amino acid sequence of GFTFSSYT (SEQ ID NO:84), a VH CDR2 comprising the amino acid sequence of TFISYDGNNK (SEQ ID NO:85), a VH CDR3 comprising the amino acid sequence of ARTGWLGPFDY (SEQ ID NO:86), and a light chain variable region comprising a VL CDR1 comprising QSVGSSY (SEQ ID NO:87), a VL CDR2 comprising the amino acid sequence of GAF, and a VL CDR3 comprising the amino acid sequence of QQYGSSPWT (SEQ ID NO:89).

In some embodiments, the anti-CTLA-4 antibody comprises a heavy chain variable region comprising a VH CDR1 comprising the amino acid sequence of GFTFSSYT (SEQ ID NO:84), or a variant thereof comprising 1, 2, 3, or 4 amino acid substitutions; a VH CDR2 comprising the amino acid sequence of TFISYDGNNK (SEQ ID NO:85), or a variant thereof comprising 1, 2, 3, or 4 amino acid substitutions; a VH CDR3 comprising the amino acid sequence of ARTGWLGPFDY (SEQ ID NO:86), or a variant thereof comprising 1, 2, 3, or 4 amino acid substitutions; and a light chain variable region comprising a VL CDR1 comprising QSVGSSY (SEQ ID NO:87), or a variant thereof comprising 1, 2, 3, or 4 amino acid substitutions; a VL CDR2 comprising the amino acid sequence of GAF, or a variant thereof comprising 1, 2, 3, or 4 amino acid substitutions; and a VL CDR3 comprising the amino acid sequence of QQYGSSPWT (SEQ ID NO:89), or a variant thereof comprising 1, 2, 3, or 4 amino acid substitutions.

In some embodiments, the anti-CTLA-4 antibody comprises a heavy chain variable region comprising a VH CDR1 comprising the amino acid sequence of GFTFSSYT (SEQ ID NO:84), a VH CDR2 comprising the amino acid sequence of TFISYDGNNK (SEQ ID NO:85), a VH CDR3 comprising the amino acid sequence of ARTGWLGPFDY (SEQ ID NO:86), and which has at least 75%, 80%, 85%, 90%, 95%, 96%, 97%, 98%, 99%, or 100% identity to the amino acid sequence of SEQ ID NO:90, and a light chain variable region comprising a VL CDR1 comprising QSVGSSY (SEQ ID NO:87), a VL CDR2 comprising the amino acid sequence of GAF, and a VL CDR3 comprising the amino acid sequence of QQYGSSPWT (SEQ ID NO:89), and which has at least 75%, 80%, 85%, 90%, 95%, 96%, 97%, 98%, 99%, or 100% identity to the amino acid sequence of SEQ ID NO:91.

In some embodiments, the anti-CTLA-4 antibody comprises a heavy chain variable region with one or more (e.g., 1, 2, or 3) substitutions, deletions, or insertions in the amino acid sequence of SEQ ID NO:90, and a light chain variable region with one or more (e.g., 1, 2, or 3) substitutions, deletions, or insertions in the amino acid sequence of SEQ ID NO:91.

In some embodiments, the anti-CTLA-4 antibody comprises a heavy chain variable region comprising the amino acid sequence of SEQ ID NO:90 and a light chain variable region comprising the amino acid sequence of SEQ ID NO:91.

In some embodiments, the anti-CTLA-4 antibody comprises a heavy chain that is at least 75%, 80%, 85%, 90%, 95%, 96%, 97%, 98%, 99%, or 100% identical to the amino acid sequence of SEQ ID NO:92 and a light chain that is at least 75%, 80%, 85%, 90%, 95%, 96%, 97%, 98%, 99%, or 100% identical to the amino acid sequence of SEQ ID NO:93.

In some embodiments, the anti-CTLA-4 antibody comprises a heavy chain comprising SEQ ID NO:92 and a light chain comprising the amino acid sequence of SEQ ID NO:93.

In some embodiments, the anti-CTLA-4 antibody comprises a heavy chain variable region comprising a VH CDR1 comprising the amino acid sequence of GFTFSSYG (SEQ ID NO:94), a VH CDR2 comprising the amino acid sequence of AVIWYDGSNK (SEQ ID NO:95), a VH CDR3 comprising the amino acid sequence of ARDPRGATLYYYYYGMDV (SEQ ID NO:96), and a light chain variable region comprising a VL CDR1 comprising QSINSY (SEQ ID NO:97), a VL CDR2 comprising the amino acid sequence of AAS, and a VL CDR3 comprising the amino acid sequence of QQYYSTPFT (SEQ ID NO:99).

In some embodiments, the anti-CTLA-4 antibody comprises a heavy chain variable region comprising a VH CDR1 comprising the amino acid sequence of GFTFSSYG (SEQ ID NO:94), or a variant thereof comprising 1, 2, 3, or 4 amino acid substitutions; a VH CDR2 comprising the amino acid sequence of AVIWYDGSNK (SEQ ID NO:95), or a variant thereof comprising 1, 2, 3, or 4 amino acid substitutions; a VH CDR3 comprising the amino acid sequence of ARDPRGATLYYYYYGMDV (SEQ ID NO:96), or a variant thereof comprising 1, 2, 3, or 4 amino acid substitutions; and a light chain variable region comprising a VL CDR1 comprising QSINSY (SEQ ID NO:97), or a variant thereof comprising 1, 2, 3, or 4 amino acid substitutions; a VL CDR2 comprising the amino acid sequence of AAS, or a variant thereof comprising 1, 2, 3, or 4 amino acid substitutions; and a VL CDR3 comprising the amino acid sequence of QQYYSTPFT (SEQ ID NO:99), or a variant thereof comprising 1, 2, 3, or 4 amino acid substitutions.

In some embodiments, the anti-CTLA-4 antibody comprises a heavy chain variable region comprising a VH CDR1 comprising the amino acid sequence of GFTFSSYG (SEQ ID NO:94), a VH CDR2 comprising the amino acid sequence of AVIWYDGSNK (SEQ ID NO:95), a VH CDR3 comprising the amino acid sequence of ARDPRGATLYYYYYGMDV (SEQ ID NO:96), and which has at least 75%, 80%, 85%, 90%, 95%, 96%, 97%, 98%, 99%, or 100% identity to the amino acid sequence of SEQ ID NO:100, and a light chain variable region comprising a VL CDR1 comprising QSINSY (SEQ ID NO:97), a VL CDR2 comprising the amino acid sequence of AAS, and a VL CDR3 comprising the amino acid sequence of QQYYSTPFT (SEQ ID NO:99), and which has at least 75%, 80%, 85%, 90%, 95%, 96%, 97%, 98%, 99%, or 100% identity to the amino acid sequence of SEQ ID NO:101.

In some embodiments, the anti-CTLA-4 antibody comprises a heavy chain variable region with one or more (e.g., 1, 2, or 3) substitutions, deletions, or insertions in the amino acid sequence of SEQ ID NO:100, and a light chain variable region with one or more (e.g., 1, 2, or 3) substitutions, deletions, or insertions in the amino acid sequence of SEQ ID NO:101.

In some embodiments, the anti-CTLA-4 antibody comprises a heavy chain variable region comprising the amino acid sequence of SEQ ID NO:100 and a light chain variable region comprising the amino acid sequence of SEQ ID NO:101.

In some embodiments, the anti-CTLA-4 antibody comprises a heavy chain that is at least 75%, 80%, 85%, 90%, 95%, 96%, 97%, 98%, 99%, or 100% identical to the amino acid sequence of SEQ ID NO:102 and a light chain that is at least 75%, 80%, 85%, 90%, 95%, 96%, 97%, 98%, 99%, or 100% identical to the amino acid sequence of SEQ ID NO:103.

In some embodiments, the anti-CTLA-4 antibody comprises a heavy chain comprising SEQ TD NO: 102 and a light chain comprising the amino acid sequence of SEQ ID NO:3.

Non-limiting examples of anti-CTLA-4 antibodies that can be used in methods described herein are provided in U.S. Pat. Nos. 7,605,238 and 6,682,736, each of which is incorporated herein by reference in its entirety. The amino acid sequence of an exemplary anti-CTLA-4 antibody, ipilimumab and tremelimumab, is described in Table 4.

TABLE 4
Anti-CTLA-4 Antibody Sequences
Ipilimumab

| Antibody | |
|---|---|
| Feature | Sequences |
| VH CDR1 | GFTFSSYT (SEQ ID NO: 84) |
| VH CDR2 | TFISYDGNNK (SEQ ID NO: 85) |
| VH CDR3 | ARTGWLGPFDY (SEQ ID NO: 86) |
| VL CDR1 | QSVGSSY (SEQ ID NO: 87) |
| VL CDR2 | GAF |
| VL CDR3 | QQYGSSPWT (SEQ ID NO: 89) |
| VH | QVQLVESGGGVVQPGRSLRLSCAASGFTFSSYTMHWVRQAPGKGLEWVTF |
| | ISYDGNNKYYADSVKGRFTISRDNSKNTLYLQMNSLRAEDTAIYYCARTG |
| | WLGPFDYWGQGTLVTVSS (SEQ ID NO: 90) |
| VL | EIVLTQSPGTLSLSPGERATLSCRASQSVGSSYLAWYQQKPGQAPRLLIY |
| | GAFSRATGIPDRESGSGSGTDETLTISRLEPEDFAVYYCQQYGSSPWTFG |
| | QGTKVEIKR (SEQ ID NO: 91) |
| HC | QVQLVESGGGVVQPGRSLRLSCAASGFTFSSYTMHWVRQAPGKGLEWVTF |
| | ISYDGNNKYYADSVKGRFTISRDNSKNTLYLQMNSLRAEDTAIYYCARTG |
| | WLGPFDYWGQGTLVTVSSASTKGPSVFPLAPSSKSTSGGTAALGCLVKDY |
| | FPEPVTVSWNSGALTSGVHTFPAVLQSSGLYSLSSVVTVPSSSLGTQTYI |
| | CNVNHKPSNTKVDKRVEPKSCDKTHTCPPCPAPELLGGPSVFLFPPKPKD |
| | TLMISRTPEVTCVVVDVSHEDPEVKFNWYVDGVEVHNAKTKPREEQYNST |
| | YRVVSVLTVLHQDWLNGKEYKCKVSNKALPAPIEKTISKAKGQPREPQVY |
| | TLPPSRDELTKNQVSLTCLVKGFYPSDIAVEWESNGQPENNYKTTPPVLD |
| | SDGSFFLYSKLTVDKSRWQQGNVFSCSVMHEALHNHYTQKSLSLSPGK |
| | (SEQ ID NO: 92) |
| LC | EIVLTQSPGTLSLSPGERATLSCRASQSVGSSYLAWYQQKPGQAPRLLIY |
| | GAFSRATGIPDRESGSGSGTDFTLTISRLEPEDFAVYYCQQYGSSPWTFG |
| | QGTKVEIKRTVAAPSVFIFPPSDEQLKSGTASVVCLLNNFYPREAKVQWK |
| | VDNALQSGNSQESVTEQDSKDSTYSLSSTLTLSKADYEKHKVYACEVTHQ |
| | GLSSPVTKSENRGEC (SEQ ID NO: 93) |

Tremelimumab

| Antibody | |
|---|---|
| Feature | Sequences |
| VH CDR1 | GFTFSSYG (SEQ ID NO: 94) |
| VH CDR2 | AVIWYDGSNK (SEQ ID NO: 95) |
| VH CDR3 | ARDPRGATLYYYYYGMDV (SEQ ID NO: 96) |
| VL CDR1 | QSINSY (SEQ ID NO: 97) |
| VL CDR2 | AAS |
| VL CDR3 | QQYYSTPFT (SEQ ID NO: 99) |
| VH | QVQLVESGGGVVQPGRSLRLSCAASGFTFSSYGMHWVRQAPGKGLEWVAV |
| | IWYDGSNKYYADSVKGRFTISRDNSKNTLYLQMNSLRAEDTAVYYCARDP |
| | RGATLYYYYYGMDVWGQGTTVTVSS (SEQ ID NO: 100) |
| VL | DIQMTQSPSSLSASVGDRVTITCRASQSINSYLDWYQQKPGKAPKLLIYA |
| | ASSLQSGVPSRESGSGSGTDFTLTISSLQPEDFATYYCQQYYSTPFTFGP |
| | GTKVEIKR (SEQ ID NO: 101) |
| HC | QVQLVESGGGVVQPGRSLRLSCAASGFTFSSYGMHWVRQAPGKGLEWVAV |
| | IWYDGSNKYYADSVKGRFTISRDNSKNTLYLQMNSLRAEDTAVYYCARDP |
| | RGATLYYYYYGMDVWGQGTTVTVSSASTKGPSVFPLAPCSRSTSESTAAL |
| | GCLVKDYFPEPVTVSWNSGALTSGVHTFPAVLQSSGLYSLSSVVTVPSSN |
| | FGTQTYTCNVDHKPSNTKVDKTVERKCCVECPPCPAPPVAGPSVFLFPPK |
| | PKDTLMISRTPEVTCVVVDVSHEDPEVQFNWYVDGVEVHNAKTKPREEQF |
| | NSTFRVVSVLTVVHQDWLNGKEYKCKVSNKGLPAPIEKTISKTKGQPREP |
| | QVYTLPPSREEMTKNQVSLTCLVKGFYPSDIAVEWESNGQPENNYKTTPP |
| | MLDSDGSFFLYSKLTVDKSRWQQGNVFSCSVMHEALHNHYTQKSLSLSPG |
| | K (SEQ ID NO: 102) |
| LC | DIQMTQSPSSLSASVGDRVTITCRASQSINSYLDWYQQKPGKAPKLLIYA |
| | ASSLQSGVPSRFSGSGSGTDFTLTISSLQPEDFATYYCQQYYSTPFTFGP |
| | GTKVEIKRTVAAPSVFIFPPSDEQLKSGTASVVCLLNNFYPREAKVQWKV |
| | DNALQSGNSQESVTEQDSKDSTYSLSSTLTLSKADYEKHKVYACEVTHQG |
| | LSSPVTKSFNRGEC (SEQ ID NO: 103) |

### Antibodies and Conjugates Thereof

Anti-GITR antibodies, anti-PD-1 antibodies, anti-PD-L1 antibodies, and anti-CTLA-4 antibodies for use in treating cancer as described herein can be whole antibodies or any antigen binding fragment (i.e., “antigen-binding portion”) or single chain thereof.

In some instances, antibodies for use in methods described herein are antibody fragments, e.g., Fab, Fab′, F(ab′)₂, Facb, and Fv.

Fragments of the antibodies may be prepared by proteolytic digestion of intact antibodies. For example, antibody fragments can be obtained by treating the whole antibody with an enzyme such as papain, pepsin, or plasmin. Papain digestion of whole antibodies produces F(ab)₂ or Fab fragments; pepsin digestion of whole antibodies yields F(ab′)₂ or Fab′; and plasmin digestion of whole antibodies yields Facb fragments.

Alternatively, antibody fragments can be produced recombinantly. For example, nucleic acids encoding the antibody fragments of interest can be constructed, introduced into an expression vector, and expressed in suitable host cells. See, e.g., Co, M. S. et al., J. Immunol., 152:2968-2976 (1994); Better, M. and Horwitz, A. H., Methods in Enzymology, 178:476-496 (1989); Plueckthun, A. and Skerra, A., Methods in Enzymology, 178:476-496 (1989); Lamoyi, E., Methods in Enzymology, 121:652-663 (1989); Rousseaux, J. et al., Methods in Enzymology, (1989) 121:663-669 (1989); and Bird, R. E. et al., TIBTECH, 9:132-137 (1991)). Antibody fragments can be expressed in and secreted from E. coli, thus allowing the facile production of large amounts of these fragments. Antibody fragments can be isolated from the antibody phage libraries. Alternatively, Fab′-SH fragments can be directly recovered from E. coli and chemically coupled to form F(ab)₂ fragments (Carter et al., Bio/Technology, 10:163-167 (1992)). According to another approach, F(ab′)₂ fragments can be isolated directly from recombinant host cell culture. Fab and F(ab′)₂ fragment with increased in vivo half-life comprising a salvage receptor binding epitope residues are described in U.S. Pat. No. 5,869,046.

In some instances, antibodies for use in methods described herein are minibodies, e.g., diabodies, single chain (scFv), and single-chain (Fv)₂ (sc(Fv)₂).

A “diabody” is a bivalent minibody constructed by gene fusion (see, e.g., Holliger, P. et al., Proc. Natl. Acad. Sci. U.S.A, 90:6444-6448 (1993); EP 404,097; WO 93/11161).

Diabodies are dimers composed of two polypeptide chains. The VL and VH domain of each polypeptide chain of the diabody are bound by linkers. The number of amino acid residues that constitute a linker can be between 2 to 12 residues (e.g., 3-10 residues or five or about five residues). The linkers of the polypeptides in a diabody are typically too short to allow the VL and VH to bind to each other. Thus, the VL and VH encoded in the same polypeptide chain cannot form a single-chain variable region fragment, but instead form a dimer with a different single-chain variable region fragment. As a result, a diabody has two antigen-binding sites.

An scFv is a single-chain polypeptide antibody obtained by linking the VH and VL with a linker (see, e.g., Huston et al., Proc. Natl. Acad. Sci. U.S.A, 85:5879-5883 (1988); and Plickthun, “The Pharmacology of Monoclonal Antibodies” Vol. 113, Ed Resenburg and Moore, Springer Verlag, New York, pp. 269-315, (1994)). The order of VHs and VLs to be linked is not particularly limited, and they may be arranged in any order. Examples of arrangements include: [VH] linker [VL]; or [VL] linker [VH]. The heavy chain variable domain and light chain variable domain in an scFv may be derived from any anti-B7-H4 antibody described herein.

An sc(Fv)₂ is a minibody in which two VHs and two VLs are linked by a linker to form a single chain (Hudson, et al., J. Immunol. Methods, (1999) 231: 177-189 (1999)). An sc(Fv)₂ can be prepared, for example, by connecting scFvs with a linker. The sc(Fv)₂ of the present invention include antibodies preferably in which two VHs and two VLs are arranged in the order of: VH, VL, VH, and VL ([VH] linker [VL] linker [VH] linker [VL]), beginning from the N terminus of a single-chain polypeptide; however the order of the two VHs and two VLs is not limited to the above arrangement, and they may be arranged in any order.

In some instances, antibodies for use in methods described herein are bispecific antibodies.

Bispecific antibodies are antibodies that have binding specificities for at least two different epitopes. Exemplary bispecific antibodies may bind to two different epitopes of the target antigen (e.g., GITR, PD-1). Other such antibodies may combine a target antigen binding site with a binding site for another protein. Bispecific antibodies can be prepared as full length antibodies or low molecular weight forms thereof (F(ab′)₂ bispecific antibodies, sc(Fv)₂ bispecific antibodies, diabody bispecific antibodies).

Traditional production of full length bispecific antibodies is based on the co-expression of two immunoglobulin heavy chain-light chain pairs, where the two chains have different specificities (Millstein et al., Nature 305:537-539 (1983)). In a different approach, antibody variable domains with the desired binding specificities are fused to immunoglobulin constant domain sequences. DNAs encoding the immunoglobulin heavy chain fusions and, if desired, the immunoglobulin light chain, are inserted into separate expression vectors, and are co-transfected into a suitable host cell. This provides for greater flexibility in adjusting the proportions of the three polypeptide fragments. It is, however, possible to insert the coding sequences for two or all three polypeptide chains into a single expression vector when the expression of at least two polypeptide chains in equal ratios results in high yields.

According to another approach described in U.S. Pat. No. 5,731,168, the interface between a pair of antibody molecules can be engineered to maximize the percentage of heterodimers that are recovered from recombinant cell culture. The preferred interface comprises at least a part of the CH3 domain. In this method, one or more small amino acid side chains from the interface of the first antibody molecule are replaced with larger side chains (e.g., tyrosine or tryptophan). Compensatory “cavities” of identical or similar size to the large side chain(s) are created on the interface of the second antibody molecule by replacing large amino acid side chains with smaller ones (e.g., alanine or threonine). This provides a mechanism for increasing the yield of the heterodimer over other unwanted end-products such as homodimers.

Bispecific antibodies include cross-linked or “heteroconjugate” antibodies. For example, one of the antibodies in the heteroconjugate can be coupled to avidin, the other to biotin. Heteroconjugate antibodies may be made using any convenient cross-linking methods.

The “diabody” technology provides an alternative mechanism for making bispecific antibody fragments. The fragments comprise a VH connected to a VL by a linker which is too short to allow pairing between the two domains on the same chain. Accordingly, the VH and VL domains of one fragment are forced to pair with the complementary VL and VH domains of another fragment, thereby forming two antigen-binding sites.

In some instances, antibodies for use in methods described herein are multivalent antibodies.

A multivalent antibody may be internalized (and/or catabolized) faster than a bivalent antibody by a cell expressing an antigen to which the antibodies bind. The antibodies describe herein can be multivalent antibodies with three or more antigen binding sites (e.g., tetravalent antibodies), which can be readily produced by recombinant expression of nucleic acid encoding the polypeptide chains of the antibody. The multivalent antibody can comprise a dimerization domain and three or more antigen binding sites. An exemplary dimerization domain comprises (or consists of) an Fc region or a hinge region. A multivalent antibody can comprise (or consist of) three to about eight (e.g., four) antigen binding sites. The multivalent antibody optionally comprises at least one polypeptide chain (e.g., at least two polypeptide chains), wherein the polypeptide chain(s) comprise two or more variable domains. For instance, the polypeptide chain(s) may comprise VD1-(X1)ₙ-VD₂-(X₂)ₙ-Fc, wherein VD1 is a first variable domain, VD2 is a second variable domain, Fc is a polypeptide chain of an Fc region, X1 and X2 represent an amino acid or peptide spacer, and n is 0 or 1.

Anti-GITR antibodies, anti-PD-1 antibodies, anti-PD-L1 antibodies, and anti-CTLA-4 antibodies for use in treating cancer as described herein can be conjugated to molecules including macromolecular substances such as polymers (e.g., polyethylene glycol (PEG), polyethylenimine (PEI) modified with PEG (PEI-PEG), polyglutamic acid (PGA) (N-(2-Hydroxypropyl) methacrylamide (HPMA) copolymers), hyaluronic acid, radioactive materials (e.g., ⁹Y, ¹³¹I), fluorescent substances, luminescent substances, haptens, enzymes, metal chelates, drugs, and toxins (e.g., calcheamicin, Pseudomonas exotoxin A, ricin (e.g., deglycosylated ricin A chain)).

In one embodiment, to improve the cytotoxic actions of anti-GITR antibodies, anti-PD-1 antibodies, anti-PD-L1 antibodies, and/or anti-CTLA-4 antibodies and consequently their therapeutic effectiveness, the antibodies are conjugated with highly toxic substances, including radioisotopes and cytotoxic agents. These conjugates can deliver a toxic load selectively to the target site (i.e., cells expressing the antigen recognized by the antibody) while cells that are not recognized by the antibody are spared. In order to minimize toxicity, conjugates are generally engineered based on molecules with a short serum half-life (thus, the use of murine sequences, and IgG3 or IgG4 isotypes).

In certain embodiments, an anti-GITR antibody, an anti-PD-1 antibody, an anti-PD-L1 antibody, and/or an anti-CTLA-4 antibody is modified with a moiety that improves its stabilization and/or retention in circulation, e.g., in blood, serum, or other tissues, e.g., by at least 1.5, 2, 5, 10, or 50 fold. For example, the anti-GITR antibody and/or the anti-PD-1 antibody can be associated with (e.g., conjugated to) a polymer, e.g., a substantially non-antigenic polymer, such as a polyalkylene oxide or a polyethylene oxide. Suitable polymers will vary substantially by weight. Polymers having molecular number average weights ranging from about 200 to about 35,000 Daltons (or about 1,000 to about 15,000, and 2,000 to about 12,500) can be used. For example, the anti-GITR antibody, the anti-PD-1 antibody, the anti-PD-L1 antibody, and/or the anti-CTLA-4 antibody can be conjugated to a water soluble polymer, e.g., a hydrophilic polyvinyl polymer, e.g., polyvinylalcohol or polyvinylpyrrolidone. Examples of such polymers include polyalkylene oxide homopolymers such as polyethylene glycol (PEG) or polypropylene glycols, polyoxyethylenated polyols, copolymers thereof and block copolymers thereof, provided that the water solubility of the block copolymers is maintained. Additional useful polymers include polyoxyalkylenes such as polyoxyethylene, polyoxypropylene, and block copolymers of polyoxyethylene and polyoxypropylene; polymethacrylates; carbomers; and branched or unbranched polysaccharides.

The above-described conjugated antibodies can be prepared by performing chemical modifications on the antibodies, respectively, or the lower molecular weight forms thereof described herein. Methods for modifying antibodies are well known in the art (e.g., U.S. Pat. Nos. 5,057,313 and 5,156,840).

### Methods of Producing Antibodies

Anti-GITR antibodies, anti-PD-1 antibodies, anti-PD-L1 antibodies, and anti-CTLA-4 antibodies for use in treating cancer as described herein can be produced using any method suitable for making antibodies.

Antibodies may be produced in bacterial or eukaryotic cells. Some antibodies, e.g., Fabs, can be produced in bacterial cells, e.g., E. coli cells. Antibodies can also be produced in eukaryotic cells such as transformed cell lines (e.g., CHO, 293E, COS). In addition, antibodies (e.g., scFvs) can be expressed in a yeast cell such as Pichia (see, e.g., Powers et al., J Immunol Methods. 251123-35 (2001)), Hansenula, or Saccharomyces. To produce the antibody of interest, a polynucleotide encoding the antibody is constructed, introduced into an expression vector, and then expressed in suitable host cells. Standard molecular biology techniques are used to prepare the recombinant expression vector, transfect the host cells, select for transformants, culture the host cells and recover the antibody.

If the antibody is to be expressed in bacterial cells (e.g., E. coli), the expression vector should have characteristics that permit amplification of the vector in the bacterial cells. Additionally, when E. coli such as JM109, DH5a, HB101, or XL1-Blue is used as a host, the vector must have a promoter, for example, a lacZ promoter (Ward et al., 341:544-546 (1989), araB promoter (Better et al., Science, 240:1041-1043 (1988)), or T7 promoter that can allow efficient expression in E. coli. Examples of such vectors include, for example, M13-series vectors, pUC-series vectors, pBR322, pBluescript, pCR-Script, pGEX-5X-1 (Pharmacia), “QIAexpress system” (QIAGEN), pEGFP, and pET (when this expression vector is used, the host is preferably BL21 expressing T7 RNA polymerase). The expression vector may contain a signal sequence for antibody secretion. For production into the periplasm of E. coli, the pelB signal sequence (Lei et al., J. Bacteriol., 169:4379 (1987)) may be used as the signal sequence for antibody secretion. For bacterial expression, calcium chloride methods or electroporation methods may be used to introduce the expression vector into the bacterial cell.

If the antibody is to be expressed in animal cells such as CHO, COS, and NIH3T3 cells, the expression vector includes a promoter necessary for expression in these cells, for example, an SV40 promoter (Mulligan et al., Nature, 277:108 (1979)), MMLV-LTR promoter, EF1α promoter (Mizushima et al., Nucleic Acids Res., 18:5322 (1990)), or CMV promoter. In addition to the nucleic acid sequence encoding the immunoglobulin or domain thereof, the recombinant expression vectors may carry additional sequences, such as sequences that regulate replication of the vector in host cells (e.g., origins of replication) and selectable marker genes. The selectable marker gene facilitates selection of host cells into which the vector has been introduced (see, e.g., U.S. Pat. Nos. 4,399,216, 4,634,665 and 5,179,017). For example, typically the selectable marker gene confers resistance to drugs, such as G418, hygromycin, or methotrexate, on a host cell into which the vector has been introduced. Examples of vectors with selectable markers include pMAM, pDR2, pBK-RSV, pBK-CMV, pOPRSV, and pOP13.

In one embodiment, antibodies are produced in mammalian cells. Exemplary mammalian host cells for expressing an antibody include Chinese Hamster Ovary (CHO cells) (including dhfr-CHO cells, described in Urlaub and Chasin (1980) Proc. Natl. Acad. Sci. USA 77:4216-4220, used with a DHFR selectable marker, e.g., as described in Kaufman and Sharp (1982) Mol. Biol. 159:601 621), human embryonic kidney 293 cells (e.g., 293, 293E, 293T), COS cells, NIH3T3 cells, lymphocytic cell lines, e.g., NSO myeloma cells and SP2 cells, and a cell from a transgenic animal, e.g., a transgenic mammal. For example, the cell is a mammary epithelial cell.

In an exemplary system for antibody expression, a recombinant expression vector encoding both the antibody heavy chain and the antibody light chain of an antibody (e.g., an anti-GITR antibody, an anti-PD-1 antibody, anti-PD-L1 antibody, or an anti-CTLA-4 antibody) is introduced into dhfr-CHO cells by calcium phosphate-mediated transfection. Within the recombinant expression vector, the antibody heavy and light chain genes are each operatively linked to enhancer/promoter regulatory elements (e.g., derived from SV40, CMV, adenovirus and the like, such as a CMV enhancer/AdMLP promoter regulatory element or an SV40 enhancer/AdMLP promoter regulatory element) to drive high levels of transcription of the genes. The recombinant expression vector also carries a DHFR gene, which allows for selection of CHO cells that have been transfected with the vector using methotrexate selection/amplification. The selected transformant host cells are cultured to allow for expression of the antibody heavy and light chains and the antibody is recovered from the culture medium.

Antibodies can also be produced by a transgenic animal. For example, U.S. Pat. No. 5,849,992 describes a method of expressing an antibody in the mammary gland of a transgenic mammal. A transgene is constructed that includes a milk-specific promoter and nucleic acids encoding the antibody of interest and a signal sequence for secretion. The milk produced by females of such transgenic mammals includes, secreted-therein, the antibody of interest. The antibody can be purified from the milk, or for some applications, used directly. Animals are also provided comprising one or more of the nucleic acids described herein.

The antibodies of the present disclosure can be isolated from inside or outside (such as medium) of the host cell and purified as substantially pure and homogenous antibodies. Methods for isolation and purification commonly used for antibody purification may be used for the isolation and purification of antibodies, and are not limited to any particular method. Antibodies may be isolated and purified by appropriately selecting and combining, for example, column chromatography, filtration, ultrafiltration, salting out, solvent precipitation, solvent extraction, distillation, immunoprecipitation, SDS-polyacrylamide gel electrophoresis, isoelectric focusing, dialysis, and recrystallization. Chromatography includes, for example, affinity chromatography, ion exchange chromatography, hydrophobic chromatography, gel filtration, reverse-phase chromatography, and adsorption chromatography (Strategies for Protein Purification and Characterization: A Laboratory Course Manual. Ed Daniel R. Marshak et al., Cold Spring Harbor Laboratory Press, 1996). Chromatography can be carried out using liquid phase chromatography such as HPLC and FPLC. Columns used for affinity chromatography include protein A column and protein G column. Examples of columns using protein A column include Hyper D, POROS, and Sepharose FF (GE Healthcare Biosciences). The present disclosure also includes antibodies that are highly purified using these purification methods.

### Methods of Treating Cancer

Aspects of the present disclosure provide methods for treating a cancer in a human subject in need thereof using an anti-GITR antibody in combination with at least one of a PD-1 inhibitor (e.g., an anti-PD-1 antibody), a PD-L1 inhibitor (e.g., an anti-PD-L1 antibody), and a CTLA-4 inhibitor (e.g., an anti-CTLA-4 antibody), e.g., the anti-GITR antibodies, anti-PD-1 antibodies, and anti-PD-L1 antibodies described herein.

### Patient Population

A human subject can be any human subject for whom diagnosis, treatment, or therapy is desired. A human subject can be of any age. In some embodiments, the human subject is at least 18 years of age.

A human subject to be treated by the methods described herein can be a human subject having, suspected of having, or at risk for having a cancer. A human subject suspected of having cancer might show one or more symptoms of cancer, e.g., unexplained weight loss, a lump or area of thickening under the skin, pain or weakness, fatigue, bleeding or bruising for no known reason, or trouble breathing. A human subject at risk for cancer can be a subject having one or more risk factors for cancer, e.g., tobacco use, alcohol consumption, age, family history, exposure to certain chemicals or radiation, or prior viral infection (e.g., human papillomavirus (HPV) infection, Epstein-Barr virus (EBV) infection). A human subject who needs the combination therapy described herein can be identified by routine medical examination, e.g., laboratory tests, biopsy, or imaging scans.

Non-limiting examples of cancer that can be treated using methods described herein include an advanced solid tumor, advanced renal cell carcinoma, anal cancer, bladder cancer, brain cancer, breast cancer, cervical cancer, colorectal cancer, endometrial cancer, esophageal cancer, gastric cancer, gastrointestinal cancer, gastroesophageal cancer, glioblastoma, head and neck squamous cell carcinoma (HNSCC), hepatocellular carcinoma (HCC), glioma, kidney cancer, liver cancer, lung cancer, lymphoma, melanoma, Merkel cell carcinoma, mesothelioma, multiple myeloma, myeloma, neuroblastoma, non-small cell lung cancer (NSCLC), ovarian cancer, pancreatic cancer, prostate cancer, rectal cancer, renal cell carcinoma (RCC), sarcoma, skin cancer, small cell lung cancer (SCLC), soft-tissue sarcoma, thoracic cancer, thyroid cancer, triple-negative breast cancer (TNBC), urothelial cancer, uterine cancer, or a combination thereof.

In some examples, a human subject to be treated by methods described herein can be a human subject having an advanced solid tumor, breast cancer, cervical cancer, colorectal cancer, endometrial cancer, esophageal cancer, gastric cancer, gastrointestinal cancer, glioblastoma, head and neck squamous cell carcinoma (HNSCC), hepatocellular carcinoma (HCC), liver cancer, lung cancer, lymphoma, melanoma, Merkel cell carcinoma, mesothelioma, non-small cell lung cancer (NSCLC), ovarian cancer, pancreatic cancer, prostate cancer, renal cell carcinoma (RCC), small cell lung cancer (SCLC), soft-tissue sarcoma, thoracic cancer, triple-negative breast cancer (TNBC), urothelial cancer, or a combination thereof.

Non-limiting examples of HNSCC that may be treated using methods described herein include, but are not limited to, oropharyngeal squamous cell carcinoma (also referred to as squamous cell carcinoma of the throat), laryngeal squamous cell carcinoma (also referred to as squamous cell carcinoma of the voice box), nasopharyngeal squamous cell carcinoma (also referred to as squamous cell carcinoma behind the nose), hypopharyngeal squamous cell carcinoma (also referred to as squamous cell carcinoma above the voice box), squamous cell carcinoma of the mouth, squamous cell carcinoma of the paranasal sinuses, and squamous cell carcinoma of the nasal cavity.

In some examples, a human subject to be treated by methods described herein can be a human subject having recurrent, unresectable, or metastatic cancer. In some embodiments, the human subject has a recurrent cancer (e.g., recurrent HNSCC). As used herein, a recurrent cancer refers to a cancer that returns following a period of time during which the cancer could not be detected in the human subject. In some embodiments, the human subject has an unresectable cancer (e.g., unresectable HNSCC). As used herein, an unresectable cancer refers to a cancer that cannot be removed by surgery. In some embodiments, the human subject has a metastatic cancer (e.g., metastatic HNSCC). As used herein, a metastatic cancer refers to a cancer in which tumor cells have spread to other organ systems such as lung, liver, skin, bone, lymph node, or combinations thereof.

In some embodiments, the human subject to be treated by the methods described herein can be a human subject having tumor cells that express GITR (e.g., GITR-expressing HNSCC tumor cells). Tumor cells can be identified as GITR-expressing tumor cells using any method known in the art, e.g., an immune assay such as immunohistochemistry (IHC) or flow cytometry.

Any of the methods described herein can further comprise a step of identifying a human subject for treatment based on presence and/or level of GITR-expressing tumor cells in the human subject (e.g., GITR-expressing HNSCC tumor cells in the human subject). In some embodiments, presence and/or level of GITR-expressing tumor cells is determined in a biological sample (e.g., a tissue sample) obtained from the human subject. In some embodiments, at least 5% (e.g., at least 10%, at least 15%, at least 20%) of tumor cells in the biological sample obtained from the human subject express GITR.

A human subject can be screened to determine whether the human subject is eligible to be treated with an anti-GITR antibody and an anti-PD-1 antibody, or an anti-GITR antibody and an anti-PD-L1 antibody, or an anti-GITR antibody and an anti-CTLA-4 antibody, or an anti-GITR antibody, an anti-PD-1 antibody, and an anti-CTLA-4 antibody. For example, a human subject who is eligible for treatment with a combination treatment described herein (e.g., an anti-GITR antibody and an anti-PD-1 antibody) does not show one or more of the following features: (a) prior treatment with an anticancer medication within the last 30 days; (b) prior treatment with a tumor necrosis factor superfamily (TNFSF) agonist; (c) presence of tumors that invade major blood vessels, as shown unequivocally by imaging studies, and with active bleeding; (d) failure to recover from toxic effects of prior therapy and/or complications from prior surgical intervention; (e) presence of one or more exclusionary laboratory values described herein; (f) brain or central nervous system (CNS) metastases, lymphoma, and/or meningitis; (g) active infection; (h) chronic systemic steroid use; (i) lung disease or pneumonitis; (j) history of organ transplant including allogeneic stem cell transplantation; (k) history or presence of an abnormal electrocardiogram (ECG); (1) a significant cardiac event within the last 6 months, acute myocardial infarction, cardiomyopathy, coronary/peripheral artery bypass graft, symptomatic congestive heart failure, critical conduction delay, cerebrovascular accident or transient ischemic attack, or pulmonary embolism; (m) vaccine use; (n) known or suspected SARS-CoV-2 infection; or (o) pregnancy or breastfeeding.

Treatment methods described herein can comprise monitoring a human subject for immune-related adverse events such as pneumonitis, diarrhea, colitis, elevated aspartate transaminase/alkaline phosphatase (AST/ALT), increased total bilirubin, hepatitis, endocrinopathies (e.g., Type 1 diabetes, hyperglycemia, hyperthyroidism, hypothyroidism, adrenal insufficiency, hypophysitis), nephritis with renal dysfunction, Stevens-Johnson syndrome (SJS), toxic epidermal necrolysis (TEN), myocarditis, or nervous system events (e.g., Guillain-Barre syndrome, autoimmune encephalitis, myasthenia gravis, autonomic neuropathy, or transverse myelitis).

Treatment methods described herein can be used on a subject that has undergone a prior anti-cancer therapy. Non-limiting examples of a prior anti-cancer therapy include an immune checkpoint inhibitor (e.g., an anti-PD-(L)1 inhibitor), a chemotherapy, or a combination thereof. In some embodiments, the subject has progressed on or after the prior anti-cancer therapy. For example, the subject has progressed on or after treatment with an anti-PD-(L)1 inhibitor.

### Administration

Methods described herein encompass administering an effective amount of an anti-GITR antibody in combination with at least one of a PD-1 inhibitor (e.g., an anti-PD-1 antibody), a PD-L1 inhibitor (e.g., an anti-PD-L1 antibody), and a CTLA-4 inhibitor (e.g., an anti-CTLA-4 antibody) to a subject, e.g., a subject in need thereof, for example, a human subject, by a variety of methods. For many applications, the route of administration is one of: intravenous injection or infusion (IV), subcutaneous injection (SC), intraperitoneally (IP), or intramuscular injection. It is also possible to use intra-articular delivery. Other modes of parenteral administration can also be used. Examples of such modes include: intraarterial, intrathecal, intracapsular, intraorbital, intracardiac, intradermal, transtracheal, subcuticular, intraarticular, subcapsular, subarachnoid, intraspinal, and epidural and intrasternal injection. In some cases, administration can be oral.

The route and/or mode of administration of an anti-GITR antibody, an anti-PD-1 antibody, an anti-PD-L1 antibody, and/or anti-CTLA-4 antibody can also be tailored for the individual case, e.g., by monitoring the subject, e.g., using tomographic imaging, e.g., to visualize a tumor.

An effective amount refers to the amount of an anti-GITR antibody and an anti-PD-1 antibody or an anti-PD-L1 antibody needed to prevent or alleviate at least one or more signs or symptoms of a cancer, and relates to a sufficient amount of an anti-GITR antibody and an anti-PD-1 antibody or an anti-PD-L1 antibody that provides the desired effect, e.g., to treat a human subject having a cancer. An effective amount also includes an amount sufficient to prevent or delay the development of a symptom of a cancer, alter the course of a symptom of a cancer (e.g., slow the progression of a symptom of a cancer), or reverse a symptom of a cancer.

An effective amount of an anti-GITR antibody can comprise a dose between 0.1 to 1000 mg, e.g., 0.5 to 1000 mg, 1 to 1000 mg, 50 to 1000 mg, 100 to 1000 mg, 150 to 1000 mg, 200 to 1000 mg, 250 to 1000 mg, 300 to 1000 mg, 350 to 1000 mg, 400 to 1000 mg, 450 to 1000 mg, 500 to 1000 mg, 550 to 1000 mg, 600 to 1000 mg, 650 to 1000 mg, 700 to 1000 mg, 750 to 1000 mg, 800 to 1000 mg, 850 to 1000 mg, 900 to 1000 mg, 950 to 1000 mg, 0.1 to 950 mg, 0.1 to 900 mg, 0.1 to 850 mg, 0.1 to 800 mg, 0.1 to 750 mg, 0.1 to 700 mg, 0.1 to 650 mg, 0.1 to 600 mg, 0.1 to 550 mg, 0.1 to 500 mg, 0.1 to 450 mg, 0.1 to 400 mg, 0.1 to 350 mg, 0.1 to 300 mg, 0.1 to 250 mg, 0.1 to 200 mg, 0.1 to 150 mg, 0.1 to 100 mg, 0.1 to 50 mg, or 0.1 to 1 mg.

In some embodiments, an effective amount of an anti-GITR antibody can comprise a dose of 0.1 mg, 0.5 mg, 1 mg, 10 mg, 25 mg, 50 mg, 75 mg, 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, or 1000 mg.

An effective amount of an anti-GITR antibody can comprise a dose between 0.3 mg/kg to 10 mg/kg, e.g., 0.5 mg/kg to 10 mg/kg, 1 mg/kg to 10 mg/kg, 2 mg/kg to 10 mg/kg, 3 mg/kg to 10 mg/kg, 4 mg/kg to 10 mg/kg, 5 mg/kg to 10 mg/kg, 6 mg/kg to 10 mg/kg, 7 mg/kg to 10 mg/kg, 8 mg/kg to 10 mg/kg, 9 mg/kg to 10 mg/kg, 0.3 mg/kg to 9 mg/kg, 0.3 mg/kg to 8 mg/kg, 0.3 mg/kg to 7 mg/kg, 0.3 mg/kg to 6 mg/kg, 0.3 mg/kg to 5 mg/kg, 0.3 mg/kg to 4 mg/kg, 0.3 mg/kg to 3 mg/kg, 0.3 mg/kg to 2 mg/kg, 0.3 mg/kg to 1 mg/kg, or 0.3 mg/kg to 0.5 mg/kg.

In some embodiments, an effective amount of an anti-GITR antibody can comprise a dose of 0.3 mg/kg, 0.5 mg/kg, 1 mg/kg, 2 mg/kg, 3 mg/kg, 4 mg/kg, 5 mg/kg, 6 mg/kg, 7 mg/kg, 8 mg/kg, 9 mg/kg, or 10 mg/kg.

In some embodiments, a human subject is administered (e.g., intravenously) an anti-GITR antibody at a dose between 0.1 to 1000 mg, e.g., 0.5 to 1000 mg, 1 to 1000 mg, 50 to 1000 mg, 100 to 1000 mg, 150 to 1000 mg, 200 to 1000 mg, 250 to 1000 mg, 300 to 1000 mg, 350 to 1000 mg, 400 to 1000 mg, 450 to 1000 mg, 500 to 1000 mg, 550 to 1000 mg, 600 to 1000 mg, 650 to 1000 mg, 700 to 1000 mg, 750 to 1000 mg, 800 to 1000 mg, 850 to 1000 mg, 900 to 1000 mg, 950 to 1000 mg, 0.1 to 950 mg, 0.1 to 900 mg, 0.1 to 850 mg, 0.1 to 800 mg, 0.1 to 750 mg, 0.1 to 700 mg, 0.1 to 650 mg, 0.1 to 600 mg, 0.1 to 550 mg, 0.1 to 500 mg, 0.1 to 450 mg, 0.1 to 400 mg, 0.1 to 350 mg, 0.1 to 300 mg, 0.1 to 250 mg, 0.1 to 200 mg, 0.1 to 150 mg, 0.1 to 100 mg, 0.1 to 50 mg, or 0.1 to 1 mg.

In some embodiments, a human subject is administered (e.g., intravenously) an anti-GITR antibody at a dose of 0.1 mg, 0.5 mg, 1 mg, 10 mg, 25 mg, 50 mg, 75 mg, 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, or 1000 mg.

In some embodiments, a human subject is administered (e.g., intravenously) an anti-GITR antibody at a dose between 0.3 mg/kg to 10 mg/kg, e.g., 0.5 mg/kg to 10 mg/kg, 1 mg/kg to 10 mg/kg, 2 mg/kg to 10 mg/kg, 3 mg/kg to 10 mg/kg, 4 mg/kg to 10 mg/kg, 5 mg/kg to 10 mg/kg, 6 mg/kg to 10 mg/kg, 7 mg/kg to 10 mg/kg, 8 mg/kg to 10 mg/kg, 9 mg/kg to 10 mg/kg, 0.3 mg/kg to 9 mg/kg, 0.3 mg/kg to 8 mg/kg, 0.3 mg/kg to 7 mg/kg, 0.3 mg/kg to 6 mg/kg, 0.3 mg/kg to 5 mg/kg, 0.3 mg/kg to 4 mg/kg, 0.3 mg/kg to 3 mg/kg, 0.3 mg/kg to 2 mg/kg, 0.3 mg/kg to 1 mg/kg, or 0.3 mg/kg to 0.5 mg/kg.

In some embodiments, a human subject is administered (e.g., intravenously) an anti-GITR antibody at a dose of 0.3 mg/kg, 0.5 mg/kg, 1 mg/kg, 2 mg/kg, 3 mg/kg, 4 mg/kg, 5 mg/kg, 6 mg/kg, 7 mg/kg, 8 mg/kg, 9 mg/kg, or 10 mg/kg.

An effective amount of an anti-PD-1 antibody can comprise a dose between 100 to 1000 mg, e.g., 150 to 1000 mg, 200 to 1000 mg, 250 to 1000 mg, 300 to 1000 mg, 350 to 1000 mg, 400 to 1000 mg, 450 to 1000 mg, 500 to 1000 mg, 550 to 1000 mg, 600 to 1000 mg, 650 to 1000 mg, 700 to 1000 mg, 750 to 1000 mg, 800 to 1000 mg, 850 to 1000 mg, 900 to 1000 mg, 950 to 1000 mg, 100 to 950 mg, 100 to 900 mg, 100 to 850 mg, 100 to 800 mg, 100 to 750 mg, 100 to 700 mg, 100 to 650 mg, 100 to 600 mg, 100 to 550 mg, 100 to 500 mg, 100 to 450 mg, 100 to 400 mg, 100 to 350 mg, 100 to 300 mg, 100 to 250 mg, 100 to 200 mg, or 100 to 150 mg.

In some embodiments, an effective amount of an anti-PD-1 antibody can comprise a dose of 100 mg, 150 mg, 200 mg, 240 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, or 1000 mg.

An effective amount of an anti-PD-1 antibody can comprise a dose between 0.5 mg/kg to 5 mg/kg, e.g., 1 mg/kg to 5 mg/kg, 1.5 mg/kg to 5 mg/kg, 2 mg/kg to 5 mg/kg, 2.5 mg/kg to 5 mg/kg, 3 mg/kg to 5 mg/kg, 3.5 mg/kg to 5 mg/kg, 4 mg/kg to 5 mg/kg, 4.5 mg/kg to 5 mg/kg, 0.5 mg/kg to 4.5 mg/kg, 0.5 mg/kg to 4 mg/kg, 0.5 mg/kg to 3.5 mg/kg, 0.5 mg/kg to 3 mg/kg, 0.5 mg/kg to 2.5 mg/kg, 0.5 mg/kg to 2 mg/kg, 0.5 mg/kg to 1.5 mg/kg, or 0.5 mg/kg to 1 mg/kg.

In some embodiments, a human subject is administered (e.g., intravenously) an anti-PD-1 antibody at a dose of 0.5 mg/kg, 1 mg/kg, 2 mg/kg, 3 mg/kg, 4 mg/kg, or 5 mg/kg.

In some embodiments, a human subject is administered (e.g., intravenously) an anti-PD-1 antibody at a dose between 100 to 1000 mg, e.g., 150 to 1000 mg, 200 to 1000 mg, 250 to 1000 mg, 300 to 1000 mg, 350 to 1000 mg, 400 to 1000 mg, 450 to 1000 mg, 500 to 1000 mg, 550 to 1000 mg, 600 to 1000 mg, 650 to 1000 mg, 700 to 1000 mg, 750 to 1000 mg, 800 to 1000 mg, 850 to 1000 mg, 900 to 1000 mg, 950 to 1000 mg, 100 to 950 mg, 100 to 900 mg, 100 to 850 mg, 100 to 800 mg, 100 to 750 mg, 100 to 700 mg, 100 to 650 mg, 100 to 600 mg, 100 to 550 mg, 100 to 500 mg, 100 to 450 mg, 100 to 400 mg, 100 to 350 mg, 100 to 300 mg, 100 to 250 mg, 100 to 200 mg, or 100 to 150 mg.

In some embodiments, a human subject is administered (e.g., intravenously) an anti-PD-1 antibody at a dose of 100 mg, 150 mg, 200 mg, 240 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, or 1000 mg.

In some embodiments, a human subject is administered (e.g., intravenously) an anti-PD-1 antibody at a dose between 0.5 mg/kg to 5 mg/kg, e.g., 1 mg/kg to 5 mg/kg, 1.5 mg/kg to 5 mg/kg, 2 mg/kg to 5 mg/kg, 2.5 mg/kg to 5 mg/kg, 3 mg/kg to 5 mg/kg, 3.5 mg/kg to 5 mg/kg, 4 mg/kg to 5 mg/kg, 4.5 mg/kg to 5 mg/kg, 0.5 mg/kg to 4.5 mg/kg, 0.5 mg/kg to 4 mg/kg, 0.5 mg/kg to 3.5 mg/kg, 0.5 mg/kg to 3 mg/kg, 0.5 mg/kg to 2.5 mg/kg, 0.5 mg/kg to 2 mg/kg, 0.5 mg/kg to 1.5 mg/kg, or 0.5 mg/kg to 1 mg/kg.

In some embodiments, a human subject is administered (e.g., intravenously) an anti-PD-1 antibody at a dose of 0.5 mg/kg, 1 mg/kg, 2 mg/kg, 3 mg/kg, 4 mg/kg, or 5 mg/kg.

Methods described herein encompass administration of an anti-GITR antibody and an anti-PD-1 antibody or an anti-PD-L1 antibody in a sequential manner, that is where each antibody is administered at a different time, as well as administration of these antibodies in a substantially simultaneous manner. When administered sequentially or substantially simultaneously, the antibodies can be administered by the same route or by different routes.

As used herein, the term “sequentially” means, unless otherwise specified, characterized by a regular sequence or order, e.g., a sequential dosage regimen could include administration of an anti-GITR antibody before, simultaneously, substantially simultaneously, or after administration of an anti-PD-1 antibody or an anti-PD-L1 antibody, but both antibodies will be administered in a regular sequence or order. The term “separate” means, unless otherwise specified, to keep apart one from the other. The term “simultaneously” means, unless otherwise specified, happening or done at the same time, i.e., the antibodies are administered at the same time. The term “substantially simultaneously” means that the antibodies are administered within minutes of each other (e.g., within 10 minutes of each other) and intends to embrace joint administration as well as consecutive administration, but if the administration is consecutive it is separated in time for only a short period (e.g., the time it would take a medical practitioner to separately administer the anti-GITR antibody and the anti-PD-1 antibody or an anti-PD-L1 antibody). As used herein, concurrent administration and substantially simultaneous administration are used interchangeably. Sequential administration refers to temporally separated administration of the anti-GITR antibody and the anti-PD-1 antibody or an anti-PD-L1 antibody.

In some embodiments, when an anti-GITR antibody and an anti-PD-1 antibody are administered sequentially, the anti-GITR antibody can be administered prior to administering the anti-PD-1 antibody. In such instances, the anti-GITR antibody can be administered between 12 to 28 days (e.g., 12 days, 13 days, 14 days, 15 days, 16 days, 17 days, 18 days, 19 days, 20 days, 21 days, 22 days, 23 days, 24 days, 25 days, 26 days, 27 days, or 28 days) prior to administering the anti-PD-1 antibody.

Methods described herein comprise administering (e.g., intravenously) to a human subject an anti-GITR antibody once every week, every 2 weeks, every 3 weeks, every 4 weeks, every 5 weeks, every 6 weeks, every 7 weeks, or every 8 weeks and administering (e.g., intravenously) to the human subject an anti-PD-1 antibody once every week, every 2 weeks, every 3 weeks, every 4 weeks, every 5 weeks, every 6 weeks, every 7 weeks, or every 8 weeks. In some embodiments, a human subject is administered (e.g., intravenously) an anti-GITR antibody once every 2 weeks and an anti-PD-1 antibody once every 4 weeks.

In some embodiments, a human subject is administered (e.g., intravenously) an anti-GITR antibody once every 2 weeks at a dose between 10 to 750 mg (e.g., a dose of 300 mg or 600 mg). In some embodiments, a human subject is administered (e.g., intravenously) an anti-GITR antibody once every 2 weeks or once every 3 weeks at a dose between 0.3 mg/kg to 10 mg/kg.

In some embodiments, a human subject is administered (e.g., intravenously) an anti-PD-1 antibody once every 2 weeks at a dose between 100 to 1000 mg (e.g., a dose of 240 mg). In some embodiments, a human subject is administered (e.g., intravenously) an anti-PD-1 antibody once every 2 weeks at a dose between 0.5 mg/kg to 5 mg/kg (e.g., a dose of 3 mg/kg). In some embodiments, a human subject is administered (e.g., intravenously) an anti-PD-1 antibody once every 3 weeks at a dose between 100 to 300 mg (e.g., a dose of 200 mg). In some embodiments, a human subject is administered (e.g., intravenously) an anti-PD-1 antibody once every 4 weeks at a dose between 100 to 1000 mg (e.g., a dose of 500 mg).

In some embodiments, a human subject is administered (e.g., intravenously) an anti-GITR antibody once every 2 weeks at a dose between 0.1 to 1000 mg (e.g., a dose of 300 mg or 600 mg) and the human subject is administered (e.g., intravenously) an anti-PD-1 antibody once every 4 weeks at a dose between 100 to 1000 mg (e.g., a dose of 500 mg). In such instances, the human subject can have HNSCC, glioblastoma, or both.

In some embodiments, a human subject is administered (e.g., intravenously) an anti-GITR antibody once every 3 weeks at a dose between 0.3 mg/kg to 10 mg/kg and the human subject is administered (e.g., intravenously) an anti-PD-1 antibody once every 3 weeks at a dose between 100 to 1000 mg (e.g., a dose of 200 mg). In such instances, the human subject can have cervical cancer, endometrial cancer, gastric cancer, esophageal cancer, hepatocellular carcinoma (HCC), melanoma, Merkel cell carcinoma, mesothelioma, non-small cell lung cancer (NSCLC), ovarian cancer, head and neck squamous cell carcinoma (HNSCC), small cell lung cancer (SCLC), renal cell carcinoma (RCC), triple-negative breast cancer (TNBC), urothelial cancer, or a combination thereof.

In some embodiments, a human subject is administered (e.g., intravenously) an anti-GITR antibody once every 2 weeks at a dose between 0.3 mg/kg to 10 mg/kg and the human subject is administered (e.g., intravenously) an anti-PD-1 antibody once every 2 weeks at a dose between 100 to 1000 mg (e.g., a dose of 240 mg) or a dose between 0.5 mg/kg to 5 mg/kg (e.g., a dose of 3 mg/kg). In such instances, the human subject can have cervical cancer, endometrial cancer, gastric cancer, esophageal cancer, hepatocellular carcinoma (HCC), melanoma, Merkel cell carcinoma, mesothelioma, non-small cell lung cancer (NSCLC), ovarian cancer, head and neck squamous cell carcinoma (HNSCC), small cell lung cancer (SCLC), renal cell carcinoma (RCC), triple-negative breast cancer (TNBC), urothelial cancer, or a combination thereof.

In some embodiments, a human subject is administered (e.g., intravenously) an anti-GITR antibody once every 3 weeks at a dose between 0.1 to 1000 mg (e.g., a dose between 10 to 750 mg) and the human subject is administered (e.g., intravenously) an anti-PD-1 antibody once every 3 weeks at a dose between 100 to 1000 mg (e.g., a dose between 100 to 300 mg). In such instances, the human subject can have colorectal cancer, melanoma, ovarian cancer, breast cancer, cervical cancer, mesothelioma, pancreatic cancer, lymphoma, or a combination thereof.

In some embodiments, a human subject is administered (e.g., intravenously) an anti-GITR antibody once every 3 weeks at a dose between 0.1 to 1000 mg (e.g., a dose between 0.12 to 60 mg) and the human subject is administered (e.g., intravenously) an anti-PD-1 antibody once every 3 weeks at a dose between 100 to 1000 mg (e.g., a dose of 200 mg). In such instances, the human subject can have colorectal cancer, melanoma, renal cell carcinoma (RCC), ovarian cancer, breast cancer, cervical cancer, head and neck squamous cell carcinoma (HNSCC), hepatocellular carcinoma (HCC), non-small cell lung cancer (NSCLC), soft-tissue sarcoma, gastric cancer, gastrointestinal cancer, mesothelioma, pancreatic cancer, urothelial cancer, or a combination thereof.

In some embodiments, a human subject is administered (e.g., intravenously) an anti-GITR antibody once every 4 weeks and the human subject is administered (e.g., intravenously) an anti-PD-1 antibody once every 4 weeks. In such instances, the human subject can have liver cancer, lung cancer, thoracic cancer, or a combination thereof.

An effective amount of an anti-PD-L1 antibody can comprise a dose between 100 to 1000 mg, e.g., 150 to 1000 mg, 200 to 1000 mg, 250 to 1000 mg, 300 to 1000 mg, 350 to 1000 mg, 400 to 1000 mg, 450 to 1000 mg, 500 to 1000 mg, 550 to 1000 mg, 600 to 1000 mg, 650 to 1000 mg, 700 to 1000 mg, 750 to 1000 mg, 800 to 1000 mg, 850 to 1000 mg, 900 to 1000 mg, 950 to 1000 mg, 100 to 950 mg, 100 to 900 mg, 100 to 850 mg, 100 to 800 mg, 100 to 750 mg, 100 to 700 mg, 100 to 650 mg, 100 to 600 mg, 100 to 550 mg, 100 to 500 mg, 100 to 450 mg, 100 to 400 mg, 100 to 350 mg, 100 to 300 mg, 100 to 250 mg, 100 to 200 mg, or 100 to 150 mg.

In some embodiments, an effective amount of an anti-PD-L1 antibody can comprise a dose of 100 mg, 150 mg, 200 mg, 240 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, or 1000 mg.

An effective amount of an anti-PD-L1 antibody can comprise a dose between 0.5 mg/kg to 5 mg/kg, e.g., 1 mg/kg to 5 mg/kg, 1.5 mg/kg to 5 mg/kg, 2 mg/kg to 5 mg/kg, 2.5 mg/kg to 5 mg/kg, 3 mg/kg to 5 mg/kg, 3.5 mg/kg to 5 mg/kg, 4 mg/kg to 5 mg/kg, 4.5 mg/kg to 5 mg/kg, 0.5 mg/kg to 4.5 mg/kg, 0.5 mg/kg to 4 mg/kg, 0.5 mg/kg to 3.5 mg/kg, 0.5 mg/kg to 3 mg/kg, 0.5 mg/kg to 2.5 mg/kg, 0.5 mg/kg to 2 mg/kg, 0.5 mg/kg to 1.5 mg/kg, or 0.5 mg/kg to 1 mg/kg.

In some embodiments, a human subject is administered (e.g., intravenously) an anti-PD-L1 antibody at a dose of 0.5 mg/kg, 1 mg/kg, 2 mg/kg, 3 mg/kg, 4 mg/kg, or 5 mg/kg.

In some embodiments, a human subject is administered (e.g., intravenously) an anti-PD-L1 antibody at a dose between 100 to 1000 mg, e.g., 150 to 1000 mg, 200 to 1000 mg, 250 to 1000 mg, 300 to 1000 mg, 350 to 1000 mg, 400 to 1000 mg, 450 to 1000 mg, 500 to 1000 mg, 550 to 1000 mg, 600 to 1000 mg, 650 to 1000 mg, 700 to 1000 mg, 750 to 1000 mg, 800 to 1000 mg, 850 to 1000 mg, 900 to 1000 mg, 950 to 1000 mg, 100 to 950 mg, 100 to 900 mg, 100 to 850 mg, 100 to 800 mg, 100 to 750 mg, 100 to 700 mg, 100 to 650 mg, 100 to 600 mg, 100 to 550 mg, 100 to 500 mg, 100 to 450 mg, 100 to 400 mg, 100 to 350 mg, 100 to 300 mg, 100 to 250 mg, 100 to 200 mg, or 100 to 150 mg.

In some embodiments, a human subject is administered (e.g., intravenously) an anti-PD-L1 antibody at a dose of 100 mg, 150 mg, 200 mg, 240 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, or 1000 mg.

In some embodiments, a human subject is administered (e.g., intravenously) an anti-PD-L1 antibody at a dose between 0.5 mg/kg to 5 mg/kg, e.g., 1 mg/kg to 5 mg/kg, 1.5 mg/kg to 5 mg/kg, 2 mg/kg to 5 mg/kg, 2.5 mg/kg to 5 mg/kg, 3 mg/kg to 5 mg/kg, 3.5 mg/kg to 5 mg/kg, 4 mg/kg to 5 mg/kg, 4.5 mg/kg to 5 mg/kg, 0.5 mg/kg to 4.5 mg/kg, 0.5 mg/kg to 4 mg/kg, 0.5 mg/kg to 3.5 mg/kg, 0.5 mg/kg to 3 mg/kg, 0.5 mg/kg to 2.5 mg/kg, 0.5 mg/kg to 2 mg/kg, 0.5 mg/kg to 1.5 mg/kg, or 0.5 mg/kg to 1 mg/kg.

In some embodiments, a human subject is administered (e.g., intravenously) an anti-PD-L1 antibody at a dose of 0.5 mg/kg, 1 mg/kg, 2 mg/kg, 3 mg/kg, 4 mg/kg, or 5 mg/kg.

In some embodiments, when an anti-GITR antibody and an anti-PD-L1 antibody are administered sequentially, the anti-GITR antibody can be administered prior to administering the anti-PD-L1 antibody. In such instances, the anti-GITR antibody can be administered between 12 to 28 days (e.g., 12 days, 13 days, 14 days, 15 days, 16 days, 17 days, 18 days, 19 days, 20 days, 21 days, 22 days, 23 days, 24 days, 25 days, 26 days, 27 days, or 28 days) prior to administering the anti-PD-L1 antibody.

Methods described herein comprise administering (e.g., intravenously) to a human subject an anti-GITR antibody once every week, every 2 weeks, every 3 weeks, every 4 weeks, every 5 weeks, every 6 weeks, every 7 weeks, or every 8 weeks and administering (e.g., intravenously) to the human subject an anti-PD-L1 antibody once every week, every 2 weeks, every 3 weeks, every 4 weeks, every 5 weeks, every 6 weeks, every 7 weeks, or every 8 weeks. In some embodiments, a human subject is administered (e.g., intravenously) an anti-GITR antibody once every 2 weeks and an anti-PD-L1 antibody once every 4 weeks.

In some embodiments, a human subject is administered (e.g., intravenously) an anti-PD-L1 antibody once every 2 weeks at a dose between 100 to 1000 mg (e.g., a dose of 240 mg). In some embodiments, a human subject is administered (e.g., intravenously) an anti-PD-L1 antibody once every 2 weeks at a dose between 0.5 mg/kg to 5 mg/kg (e.g., a dose of 3 mg/kg). In some embodiments, a human subject is administered (e.g., intravenously) an anti-PD-L1 antibody once every 3 weeks at a dose between 100 to 300 mg (e.g., a dose of 200 mg). In some embodiments, a human subject is administered (e.g., intravenously) an anti-PD-L1 antibody once every 4 weeks at a dose between 100 to 1000 mg (e.g., a dose of 500 mg).

In some embodiments, a human subject is administered (e.g., intravenously) an anti-GITR antibody once every 2 weeks at a dose between 0.1 to 1000 mg (e.g., a dose of 300 mg or 600 mg) and the human subject is administered (e.g., intravenously) an anti-PD-L1 antibody once every 4 weeks at a dose between 100 to 1000 mg (e.g., a dose of 500 mg). In such instances, the human subject can have HNSCC, glioblastoma, or both.

In some embodiments, a human subject is administered (e.g., intravenously) an anti-GITR antibody once every 3 weeks at a dose between 0.3 mg/kg to 10 mg/kg and the human subject is administered (e.g., intravenously) an anti-PD-L1 antibody once every 3 weeks at a dose between 100 to 1000 mg (e.g., a dose of 200 mg). In such instances, the human subject can have cervical cancer, endometrial cancer, gastric cancer, esophageal cancer, hepatocellular carcinoma (HCC), melanoma, Merkel cell carcinoma, mesothelioma, non-small cell lung cancer (NSCLC), ovarian cancer, head and neck squamous cell carcinoma (HNSCC), small cell lung cancer (SCLC), renal cell carcinoma (RCC), triple-negative breast cancer (TNBC), urothelial cancer, or a combination thereof.

In some embodiments, a human subject is administered (e.g., intravenously) an anti-GITR antibody once every 2 weeks at a dose between 0.3 mg/kg to 10 mg/kg and the human subject is administered (e.g., intravenously) an anti-PD-L1 antibody once every 2 weeks at a dose between 100 to 1000 mg (e.g., a dose of 240 mg) or a dose between 0.5 mg/kg to 5 mg/kg (e.g., a dose of 3 mg/kg). In such instances, the human subject can have cervical cancer, endometrial cancer, gastric cancer, esophageal cancer, hepatocellular carcinoma (HCC), melanoma, Merkel cell carcinoma, mesothelioma, non-small cell lung cancer (NSCLC), ovarian cancer, head and neck squamous cell carcinoma (HNSCC), small cell lung cancer (SCLC), renal cell carcinoma (RCC), triple-negative breast cancer (TNBC), urothelial cancer, or a combination thereof.

In some embodiments, a human subject is administered (e.g., intravenously) an anti-GITR antibody once every 3 weeks at a dose between 0.1 to 1000 mg (e.g., a dose between 10 to 750 mg) and the human subject is administered (e.g., intravenously) an anti-PD-L1 antibody once every 3 weeks at a dose between 100 to 1000 mg (e.g., a dose between 100 to 300 mg). In such instances, the human subject can have colorectal cancer, melanoma, ovarian cancer, breast cancer, cervical cancer, mesothelioma, pancreatic cancer, lymphoma, or a combination thereof.

In some embodiments, a human subject is administered (e.g., intravenously) an anti-GITR antibody once every 3 weeks at a dose between 0.1 to 1000 mg (e.g., a dose between 0.12 to 60 mg) and the human subject is administered (e.g., intravenously) an anti-PD-L1 antibody once every 3 weeks at a dose between 100 to 1000 mg (e.g., a dose of 200 mg). In such instances, the human subject can have colorectal cancer, melanoma, renal cell carcinoma (RCC), ovarian cancer, breast cancer, cervical cancer, head and neck squamous cell carcinoma (HNSCC), hepatocellular carcinoma (HCC), non-small cell lung cancer (NSCLC), soft-tissue sarcoma, gastric cancer, gastrointestinal cancer, mesothelioma, pancreatic cancer, urothelial cancer, or a combination thereof.

In some embodiments, a human subject is administered (e.g., intravenously) an anti-GITR antibody once every 4 weeks and the human subject is administered (e.g., intravenously) an anti-PD-L1 antibody once every 4 weeks. In such instances, the human subject can have liver cancer, lung cancer, thoracic cancer, or a combination thereof.

An effective amount of an anti-CTLA-4 antibody can comprise a dose between 100 to 1000 mg, e.g., 150 to 1000 mg, 200 to 1000 mg, 250 to 1000 mg, 300 to 1000 mg, 350 to 1000 mg, 400 to 1000 mg, 450 to 1000 mg, 500 to 1000 mg, 550 to 1000 mg, 600 to 1000 mg, 650 to 1000 mg, 700 to 1000 mg, 750 to 1000 mg, 800 to 1000 mg, 850 to 1000 mg, 900 to 1000 mg, 950 to 1000 mg, 100 to 950 mg, 100 to 900 mg, 100 to 850 mg, 100 to 800 mg, 100 to 750 mg, 100 to 700 mg, 100 to 650 mg, 100 to 600 mg, 100 to 550 mg, 100 to 500 mg, 100 to 450 mg, 100 to 400 mg, 100 to 350 mg, 100 to 300 mg, 100 to 250 mg, 100 to 200 mg, or 100 to 150 mg.

In some embodiments, an effective amount of an anti-CTLA-4 antibody can comprise a dose of 100 mg, 150 mg, 200 mg, 240 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, or 1000 mg.

An effective amount of an anti-CTLA-4 antibody can comprise a dose between 0.5 mg/kg to 5 mg/kg, e.g., 1 mg/kg to 5 mg/kg, 1.5 mg/kg to 5 mg/kg, 2 mg/kg to 5 mg/kg, 2.5 mg/kg to 5 mg/kg, 3 mg/kg to 5 mg/kg, 3.5 mg/kg to 5 mg/kg, 4 mg/kg to 5 mg/kg, 4.5 mg/kg to 5 mg/kg, 0.5 mg/kg to 4.5 mg/kg, 0.5 mg/kg to 4 mg/kg, 0.5 mg/kg to 3.5 mg/kg, 0.5 mg/kg to 3 mg/kg, 0.5 mg/kg to 2.5 mg/kg, 0.5 mg/kg to 2 mg/kg, 0.5 mg/kg to 1.5 mg/kg, or 0.5 mg/kg to 1 mg/kg.

In some embodiments, a human subject is administered (e.g., intravenously) an anti-CTLA-4 antibody at a dose of 0.5 mg/kg, 1 mg/kg, 2 mg/kg, 3 mg/kg, 4 mg/kg, or 5 mg/kg. In some embodiments, a human subject is administered (e.g., intravenously) an anti-CTLA-4 antibody at a dose between 100 to 1000 mg, e.g., 150 to 1000 mg, 200 to 1000 mg, 250 to 1000 mg, 300 to 1000 mg, 350 to 1000 mg, 400 to 1000 mg, 450 to 1000 mg, 500 to 1000 mg, 550 to 1000 mg, 600 to 1000 mg, 650 to 1000 mg, 700 to 1000 mg, 750 to 1000 mg, 800 to 1000 mg, 850 to 1000 mg, 900 to 1000 mg, 950 to 1000 mg, 100 to 950 mg, 100 to 900 mg, 100 to 850 mg, 100 to 800 mg, 100 to 750 mg, 100 to 700 mg, 100 to 650 mg, 100 to 600 mg, 100 to 550 mg, 100 to 500 mg, 100 to 450 mg, 100 to 400 mg, 100 to 350 mg, 100 to 300 mg, 100 to 250 mg, 100 to 200 mg, or 100 to 150 mg.

In some embodiments, a human subject is administered (e.g., intravenously) an anti-CTLA-4 antibody at a dose of 100 mg, 150 mg, 200 mg, 240 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, or 1000 mg.

In some embodiments, a human subject is administered (e.g., intravenously) an anti-CTLA-4 antibody at a dose between 0.5 mg/kg to 5 mg/kg, e.g., 1 mg/kg to 5 mg/kg, 1.5 mg/kg to 5 mg/kg, 2 mg/kg to 5 mg/kg, 2.5 mg/kg to 5 mg/kg, 3 mg/kg to 5 mg/kg, 3.5 mg/kg to 5 mg/kg, 4 mg/kg to 5 mg/kg, 4.5 mg/kg to 5 mg/kg, 0.5 mg/kg to 4.5 mg/kg, 0.5 mg/kg to 4 mg/kg, 0.5 mg/kg to 3.5 mg/kg, 0.5 mg/kg to 3 mg/kg, 0.5 mg/kg to 2.5 mg/kg, 0.5 mg/kg to 2 mg/kg, 0.5 mg/kg to 1.5 mg/kg, or 0.5 mg/kg to 1 mg/kg.

In some embodiments, a human subject is administered (e.g., intravenously) an anti-CTLA-4 antibody at a dose of 0.5 mg/kg, 1 mg/kg, 2 mg/kg, 3 mg/kg, 4 mg/kg, or 5 mg/kg.

In some embodiments, when an anti-GITR antibody and an anti-CTLA-4 antibody are administered sequentially, the anti-GITR antibody can be administered prior to administering the anti-CTLA-4 antibody. In such instances, the anti-GITR antibody can be administered between 12 to 28 days (e.g., 12 days, 13 days, 14 days, 15 days, 16 days, 17 days, 18 days, 19 days, 20 days, 21 days, 22 days, 23 days, 24 days, 25 days, 26 days, 27 days, or 28 days) prior to administering the anti-CTLA-4 antibody.

Methods described herein comprise administering (e.g., intravenously) to a human subject an anti-GITR antibody once every week, every 2 weeks, every 3 weeks, every 4 weeks, every 5 weeks, every 6 weeks, every 7 weeks, or every 8 weeks and administering (e.g., intravenously) to the human subject an anti-CTLA-4 antibody once every week, every 2 weeks, every 3 weeks, every 4 weeks, every 5 weeks, every 6 weeks, every 7 weeks, or every 8 weeks. In some embodiments, a human subject is administered (e.g., intravenously) an anti-GITR antibody once every 2 weeks and an anti-CTLA-4 antibody once every 3 weeks or once every 6 weeks.

In some embodiments, a human subject is administered (e.g., intravenously) an anti-GITR antibody once every 2 weeks at a dose between 10 to 750 mg (e.g., a dose of 300 mg or 600 mg). In some embodiments, a human subject is administered (e.g., intravenously) an anti-GITR antibody once every 2 weeks or once every 3 weeks at a dose between 0.3 mg/kg to 10 mg/kg.

In some embodiments, a human subject is administered (e.g., intravenously) an anti-CTLA-4 antibody once every 6 weeks at a dose between 100 to 1000 mg (e.g., a dose of 500 mg). In some embodiments, a human subject is administered (e.g., intravenously) an anti-CTLA-4 antibody once every 6 weeks at a dose between 0.5 mg/kg to 5 mg/kg (e.g., a dose of 3 mg/kg). In some embodiments, a human subject is administered (e.g., intravenously) an anti-CTLA-4 antibody once every 6 weeks at a dose between 100 to 300 mg (e.g., a dose of 200 mg). In some embodiments, a human subject is administered (e.g., intravenously) an anti-CTLA-4 antibody once every 3 weeks at a dose between 100 to 1000 mg (e.g., a dose of 500 mg).

In some embodiments, a human subject is administered (e.g., intravenously) an anti-GITR antibody once every 2 weeks at a dose between 0.1 to 1000 mg (e.g., a dose of 300 mg or 600 mg) and the human subject is administered (e.g., intravenously) an anti-CTLA-4 antibody once every 6 weeks at a dose between 100 to 1000 mg (e.g., a dose of 500 mg). In such instances, the human subject can have HNSCC, glioblastoma, or both.

In some embodiments, a human subject is administered (e.g., intravenously) an anti-GITR antibody once every 3 weeks at a dose between 0.3 mg/kg to 10 mg/kg and the human subject is administered (e.g., intravenously) an anti-CTLA-4 antibody once every 6 weeks at a dose between 100 to 1000 mg (e.g., a dose of 200 mg). In such instances, the human subject can have cervical cancer, endometrial cancer, gastric cancer, esophageal cancer, hepatocellular carcinoma (HCC), melanoma, Merkel cell carcinoma, mesothelioma, non-small cell lung cancer (NSCLC), ovarian cancer, head and neck squamous cell carcinoma (HNSCC), small cell lung cancer (SCLC), renal cell carcinoma (RCC), triple-negative breast cancer (TNBC), urothelial cancer, or a combination thereof.

In some embodiments, a human subject is administered (e.g., intravenously) an anti-GITR antibody once every 2 weeks at a dose between 0.3 mg/kg to 10 mg/kg and the human subject is administered (e.g., intravenously) an anti-CTLA-4 antibody once every 6 weeks at a dose between 100 to 1000 mg (e.g., a dose of 240 mg) or a dose between 0.5 mg/kg to 5 mg/kg (e.g., a dose of 3 mg/kg). In such instances, the human subject can have cervical cancer, endometrial cancer, gastric cancer, esophageal cancer, hepatocellular carcinoma (HCC), melanoma, Merkel cell carcinoma, mesothelioma, non-small cell lung cancer (NSCLC), ovarian cancer, head and neck squamous cell carcinoma (HNSCC), small cell lung cancer (SCLC), renal cell carcinoma (RCC), triple-negative breast cancer (TNBC), urothelial cancer, or a combination thereof.

In some embodiments, a human subject is administered (e.g., intravenously) an anti-GITR antibody once every 3 weeks at a dose between 0.1 to 1000 mg (e.g., a dose between 10 to 750 mg, e.g., 300 mg) and the human subject is administered (e.g., intravenously) an anti-CTLA-4 antibody once every 3 weeks at a dose between 100 to 1000 mg (e.g., a dose between 100 to 300 mg). In such instances, the human subject can have colorectal cancer, melanoma, ovarian cancer, breast cancer, cervical cancer, mesothelioma, pancreatic cancer, lymphoma, or a combination thereof.

In some embodiments, a human subject is administered (e.g., intravenously) an anti-GITR antibody once every 3 weeks at a dose between 0.1 to 1000 mg (e.g., a dose between 0.12 to 60 mg) and the human subject is administered (e.g., intravenously) an anti-CTLA-4 antibody once every 6 weeks at a dose between 100 to 1000 mg (e.g., a dose of 200 mg). In such instances, the human subject can have colorectal cancer, melanoma, renal cell carcinoma (RCC), ovarian cancer, breast cancer, cervical cancer, head and neck squamous cell carcinoma (HNSCC), hepatocellular carcinoma (HCC), non-small cell lung cancer (NSCLC), soft-tissue sarcoma, gastric cancer, gastrointestinal cancer, mesothelioma, pancreatic cancer, urothelial cancer, or a combination thereof.

In some embodiments, a human subject is administered (e.g., intravenously) an anti-GITR antibody once every 4 weeks and the human subject is administered (e.g., intravenously) an anti-CTLA-4 antibody once every 6 weeks. In such instances, the human subject can have melanoma, cervical cancer, gastric cancer, head and neck squamous cell carcinoma (HNSCC), or a combination thereof.

Methods described herein encompass repeated administrations of an anti-GITR antibody and an anti-PD-1 antibody or an anti-PD-L1 antibody and/or an anti-CTLA-4 antibody. For repeated administrations over several days or longer, depending on the condition, the treatment can be sustained until a desired suppression of symptoms occurs or until sufficient therapeutic levels are achieved to alleviate a cancer, or a symptom thereof.

Methods described herein encompass administration of the combination therapy described herein and an additional anti-cancer therapy. Non-limiting examples of an additional anti-cancer therapy include an immune checkpoint inhibitor (e.g., an anti-CTLA-4 antibody such as ipilimumab) and an indoleamine 2,3-dioxygenase-1 (IDO1) inhibitor such as epacadostat.

In some embodiments, certain medications or vaccinations are excluded from use in methods for treating HNSCC described herein. For example, in some embodiments, methods described herein do not comprise administration of a cancer vaccine to the human subject.

Non-limiting examples of a cancer vaccine that can be excluded from methods described herein include a multivalent autophagosome cancer vaccine, a dendritic cell vaccine, an antigen vaccine, an anti-idiotype vaccine, a DNA vaccine, a tumor cell vaccine, and a combination thereof.

The following examples are provided to better illustrate the claimed invention and are not to be interpreted as limiting the scope of the invention in any way. To the extent that specific materials are mentioned, it is merely for purposes of illustration and is not intended to limit the invention. One skilled in the art can develop equivalent means or reactants without the exercise of inventive capacity and without departing from the scope of the invention.

### EXAMPLES

### Example 1: A Study of an Anti-GITR Antibody in Combination with an Anti-PD-1 Antibody in Subjects with Recurrent or Metastatic Head and Neck Squamous Cell Carcinoma

This is a Phase 2, open-label, multicenter study of an anti-GITR antibody (Anti-GITR Antibody A, as described in Table 1) in combination with an anti-PD-1 antibody (retifanlimab, as described in Table 2) in subjects with recurrent or metastatic head and neck squamous cell carcinoma (r/m HNSCC).

### 1. Protocol Summary

### Overall Design:

This study will evaluate the safety and preliminary efficacy of Anti-GITR Antibody A in combination with retifanlimab in subjects with recurrent or metastatic HNSCC, whose tumors express high GITR levels (defined as tumor positive score (TPS) ≥10%).

### Treatment Groups and Duration:

Part 1 (lead-in): Combination treatment of Anti-GITR Antibody A plus retifanlimab for up to 2 years in subjects who have been previously treated with anti-PD-(L)1 therapy.
   Cohort 1: Anti-GITR Antibody A 300 mg every two weeks (Q2W) with retifanlimab 500 mg every 4 weeks (Q4W).
   Cohort 2: Anti-GITR Antibody A 600 mg Q2W with retifanlimab 500 mg Q4W.
Part 2 (expansion): Combination treatment for up to 2 years of Anti-GITR Antibody A at the recommended Phase 2 dose (RP2D) when given with retifanlimab as per the safety results in Part 1.
   Treatment Group A: Anti-GITR Antibody A and retifanlimab combination in subjects who have been previously treated with anti-PD-(L)1 therapy.
   Treatment Group B (exploratory cohort): Anti-GITR Antibody A and retifanlimab combination in subjects who are naive to anti-PD-(L)1 therapy.

### Justification for Dose

For Part 1 (lead-in), the starting dose of Anti-GITR Antibody A will be 300 mg Q2W, which is the recommended Phase 2 dose (RP2D) of Anti-GITR Antibody A given in combination with nivolumab (anti-PD-1 mAb) in participants with advanced/metastatic cancer. The second dose level of Anti-GITR Antibody A will be 600 mg Q2W, which was selected based on safety data and exposure-response analyses obtained in earlier clinical studies with Anti-GITR Antibody A.

Anti-GITR Antibody A has been evaluated as monotherapy at dose regimens of 0.03 to 20.0 mg/kg Q2W, 400 mg Q4W, and 300 mg Q2W (RP2D) in a prior study. Anti-GITR Antibody A was generally well tolerated at all doses tested, and an MTD was not reached. In addition, when administered in combination with the PD-1 inhibitor nivolumab in a prior study, Anti-GITR Antibody A was generally well tolerated at doses up to 10 mg/kg Q2W (i.e., the highest dose tested in combination with nivolumab). Based on receptor occupancy assessed with samples from participants enrolled in the dose-escalation cohorts (3-4 participants per dose cohort) of the Anti-GITR Antibody A monotherapy study, it was determined that all participants receiving doses ≥5 mg presented saturated receptor occupancy postinfusion, and the average receptor occupancy was maintained above 90% at trough. Doses analyzed with receptor occupancy include 0.03, 0.1, 0.3, 1, 3, 5, 10, and 20 mg/kg. A flat dose of 600 mg is comparable to 10 mg/kg, and is within the range of receptor saturation. Based on relationship analysis between serum concentration of Anti-GITR Antibody A and GITR receptor occupancy, doses ≥5 mg/kg Q2W would insure a dose that consistently provides ≥90% saturated receptors and adequately avoids ADAs.

86 participants in a prior study have received Anti-GITR Antibody A at the RP2D of 300 mg Q2W in combination with nivolumab 240 mg Q2W. Preliminary data of this combination treatment have shown an ORR of 31.1% (14/45 participants) in participants with advanced HNSCC who progressed on prior treatment with a platinum-containing regimen. In addition, exposure-response analyses suggest that higher Anti-GITR Antibody A exposure is found in responders as compared with nonresponders in this cohort. Therefore, based on preliminary population PK modeling, the dose of Anti-GITR Antibody A 600 mg Q2W may achieve exposure in the range to the responders, providing further justification for the evaluation of this dose in combination with retifanlimab in this study.

The flat dose of 600 mg Q2W of Anti-GITR Antibody A translates to a weight-based dose of approximately 8.5 mg/kg Q2W, which is within the range of doses found safe and tolerable as single-agent and combination treatment with a PD-1 inhibitor in prior studies.

Retifanlimab will be administered at 500 mg Q4W. The selection of this dose was based on safety and modeling of clinical PK data from a FI monotherapy study in which 219 participants were treated with body weight-based or flat-dosing at doses of 1 mg/kg Q2W, 3 mg/kg Q2W, 3 mg/kg Q4W, 10 mg/kg Q2W, 10 mg/kg Q4W, 375 mg Q3W, 500 mg Q4W, and 750 mg Q4W.

Pharmacokinetic data from the prior study suggested that retifanlimab exposure increased in a dose-dependent manner with doses ranging from 1 to 10 mg/kg (including flat doses of 375 to 750 mg). The PK of retifanlimab following IV administration was well characterized by a 2-compartment linear disposition model with time-dependent elimination. A population PK model estimated typical retifanlimab clearance as 0.0127 L/h with a steady-state half-life of 18.4 days. Similar PK profiles were observed for the 3 mg/kg Q2W and 500 mg Q4W doses. In addition, full PD-1 receptor occupancy was observed on PD-1 expressing CD4+ and CD8+ cells with effects on circulating cytokines that are typical for a PD-1 inhibitor in all dose regimens of the prior study. Therefore, the 500 mg Q4W dose of retifanlimab was selected as a dose for further development.

### 2. Study Purpose

The purpose of the Phase 2 study is to investigate the safety, tolerability, efficacy, PK, and pharmacodynamics of Anti-GITR Antibody A given as combination treatment with retifanlimab in subjects with recurrent/metastatic ENSCC and GTR-positive tumors (defined as TPS≥10%).

A safety lead-in (Part 1) will be followed by an expansion (Part 2) at the selected dose based on the safety data obtained in Part 1. An interim futility analysis will be performed once 17 subjects have been enrolled in the study. The futility analysis will use a Bayesian predictive probability approach to avoid unnecessary exposure of subjects to a potentially ineffective treatment.

### 3. Objectives and Endpoints

### Objectives and Endpoints:

Table 5 presents the primary and secondary objectives and endpoints.

TABLE 5
Objectives and Endpoints

| | |
|---|---|
| Objectives | Endpoints |

Primary

| | |
|---|---|
| Part 1 (lead-in; Cohorts 1 and 2): To | Safety and tolerability assessed by monitoring |
| determine the safety and tolerability of Anti- | the frequency and severity of adverse events |
| GITR Antibody A in combination with | (AEs) through physical examinations, |
| retifanlimab. | evaluations of vital signs, ECGs, and laboratory |
| | abnormalities. |
| To determine the preliminary efficacy of Anti- | ORR, defined as the percentage of subjects |
| GITR Antibody A at the selected dose in | having a CR or PR as determined by |
| combination with retifanlimab in all subjects | investigator assessment of radiographic disease |
| who have been previously treated with | per Response Evaluation Criteria in Solid |
| anti-PD-(L)1 therapy (Part 1 Cohort 1 or 2 and | Tumors (RECIST v1.1). (ORR, objective |
| Part 2 Treatment Group A). | response rate; CR, complete response; PR, |
| | partial response). |

Secondary

| | |
|---|---|
| To determine the preliminary efficacy of Anti- | DOR, defined as the time from the earliest |
| GITR Antibody A at the selected dose in | date of CR or PR, as determined by |
| combination with retifanlimab in all subjects | investigator assessment of radiographic |
| who have been previously treated with | disease per RECIST v1.1, until disease |
| anti-PD-(L)1 therapy (Part 1 Cohort 1 or 2 and | progression or death due to any cause, |
| Part 2 Treatment Group A). | whichever occurs first. |
| | DCR, defined as the percentage of subjects |
| | having CR, PR, or SD, as determined by |
| | investigator assessment of radiographic |
| | disease per RECIST v1.1. (DOR, duration of |
| | response; DCR, disease control rate). |
| | PFS, defined as the time from the treatment |
| | start date until the earliest date of disease |
| | progression, as determined by investigator |
| | assessment of radiographic disease per |
| | RECIST v1.1, or death due to any cause, |
| | whichever occurs first. (PFS, progression-free |
| | survival). |
| To further determine the safety and tolerability | Safety and tolerability assessed by monitoring |
| of Anti-GITR Antibody A in combination with | the frequency and severity of AEs through |
| retifanlimab in all subjects who have been | physical examinations, evaluations of vital |
| previously treated with anti-PD-(L)1 therapy | signs, ECGs, and laboratory abnormalities. |
| (Part 1 Cohort 1 or 2 and Part 2 Treatment | |
| Group A) and subjects who are naive to anti- | |
| PD-(L)1 therapy (Part 2 Treatment Group B). | |

Exploratory endpoints include pharmacokinetics, pharmacodynamics, and overall survival assessments of Anti-GITR Antibody A in combination with retifanlimab.

### 4. Study Design

### 4.1 Overall Design

This is a multicenter, open-label, single-arm, Phase 2, clinical study to investigate the safety, tolerability, efficacy, PK, and pharmacodynamics of Anti-GITR Antibody A given in combination with retifanlimab in subjects with recurrent or metastatic HNSCC who have progressed on or after prior systemic treatment (including anti-PD-(L)1 therapy, as applicable) and have GITR-positive tumors (defined as TPS≥10%).

The study will consist of 2 parts, a safety lead-in part (Part 1) followed by a dose expansion part (Part 2).

### 4.1.1. Part 1 (Lead-in)

Part 1 (lead-in) will consist of a dose escalation and will determine the tolerability and safety of Anti-GITR Antibody A given in combination with retifanlimab in subjects who have been previously treated with anti-PD-(L)1 therapy. In addition, the predictability of the tumor GITR biomarker (TPS≥10% cutoff), translational assessments of pharmacodynamic markers, and preliminary antitumor activity of the combination treatment will be evaluated.

Up to 12 evaluable subjects will be enrolled and treated at 2 planned dose levels of Anti-GITR Antibody A in Cohorts 1 and 2 as follows:
Cohort 1 (Dose Level 1): The starting dose of Anti-GITR Antibody A will be 300 mg Q2W.
Cohort 2 (Dose Level 2): The second dose level of the anti-GITR antibody will be 600 mg Q2W.

Dose escalation will follow the Bayesian optimal interval (BOIN) design algorithm (Yuan et al., Clin Cancer Res 2016; 22:4291-4301). Given the maximum target dose-limiting toxicity (DLT) rate of 33% for Anti-GITR Antibody A in combination with retifanlimab, the decision boundary rules are shown in Table 6. The BOIN design also includes an elimination rule. When ≥3 subjects have been treated, if the probability that the estimated toxicity rate that is above the target DLT rate is >95% at a certain dose level, then this dose level and higher dose levels are assumed to be too toxic and will be eliminated. If the lowest dose level is eliminated, then the whole dose escalation will be terminated. Table 6 (bottom row) provides the elimination rules. Based on this algorithm, a minimum of 3 evaluable subjects and a maximum of 6 evaluable subjects will be enrolled at each tested dose level. The dose escalation will continue, based on the rules in Table 6, until at least 1 of the following occurs:
Enrollment of additional subjects at a dose level that already has 6 evaluable subjects.
Dose escalation to a dose level that has already been eliminated.
Dose escalation above 600 mg Q2W.

At that point, the dose escalation will be stopped.

TABLE 6
Decision Boundaries for Target Dose-
Limiting Toxicity Rate of 33% in Part 1

| | Number of Evaluable Subjects Treated at the | | | | | |
|---|---|---|---|---|---|---|
| | Current Anti-GITR Antibody Dose Level | | | | | |
| Action | 1 | 2 | 3 | 4 | 5 | 6^{a} |
| Escalate if # of DLTs≤ | NA | NA | 0 | 1 | 1 | 1 |
| De-escalate if # of | NA | NA | 2 | 2 | 2 | 3 |
| DLTs≥ | | | | | | |
| Elimination if # of | NA | NA | 3 | 3 | 4 | 4 |
| DLTs≥ | | | | | | |

^{a}If 6 evaluable subjects are enrolled at a dose level, and 2 of those subjects experience a DLT, then the medical monitor and the investigators will review the entirety of the data and decide whether to escalate the dose level, de-escalate the dose level, or stop at that dose level.

Anti-GITR Antibody A dose will be escalated using an open-label BOIN design and a pharmacologically active dose (PAD) or the maximum tolerated dose (MTD) will be determined, or the maximum dose of Anti-GITR Antibody A (600 mg Q2W) will be reached. The MTD is the maximum tolerated or tested dose of Anti-GITR Antibody A, such that fewer than 33% of the subjects receiving the combination experience a DLT during the first 28 days on study treatment. After the dose escalation is completed, one of the Anti-GITR Antibody A dose levels that is pharmacologically active and tolerable in combination with retifanlimab (e.g., MTD or lower), will be selected as the RP2D.

### 4.1.2. Part 2 (Expansion)

Part 2 (expansion) will determine the efficacy of Anti-GITR Antibody A Q2W at the RP2D when given in combination with retifanlimab, as per the results obtained in Part 1 (lead-in). Preliminary PK, pharmacodynamic, and additional antitumor activity criteria will also be evaluated.

Up to 38 evaluable subjects will be enrolled in 2 treatment groups:
Treatment Group A will enroll up to 32 subjects who have been previously treated with anti-PD-(L)1 therapy. The ORR data from the 3 to 6 subjects treated at the recommended dose in Part 1 will be included for the interim and analyses planned in Part 2. A total of 35 subjects who have been previously treated with anti-PD-(L)1 therapy will be used for the final analyses.
Treatment Group B will enroll 6 subjects who are naive to anti-PD-(L)1 therapy.

A total of up to 50 subjects are planned to be enrolled in the study. An interim futility analysis using a Bayesian predictive probability approach will be performed during Part 2 (expansion) after study treatment has been administered to 17 subjects (from Part 1 and Part 2) previously treated with anti-PD-(L)1 therapy, and their responses assessed.
If <3 confirmed responses are observed, then enrollment will be discontinued. Otherwise, the additional subjects will be enrolled into the study. Enrollment will be held during the interim analysis.
If there are ≥3 confirmed responses observed before 17 subjects have been enrolled, then the study can directly continue with enrollment of the additional subjects.

Mandatory pretreatment tumor biopsies (fresh and/or archival, as applicable) will be collected and analyzed for GITR expression as part of the eligibility assessment at screening; subjects with GITR-positive tumors (defined as TPS≥10%) will be eligible for enrollment in the study if they fulfill all other eligibility criteria. Mandatory on-treatment biopsies will also be collected from all subjects to evaluate changes in the tumor and tumor microenvironment (TME) and the correlation with clinical outcomes.

Tumor assessments by site investigator review according to RECIST v1.1 will be performed at baseline and subsequently every 8 weeks for the first year of treatment and every 12 weeks thereafter. The investigator will use immune Response Criteria in Solid Tumors (iRECIST) to assess tumor response to guide treatment decisions for discontinuation of therapy due to radiographic disease progression for subjects that are clinically stable.

Safety will be evaluated from the time the participant signs the informed consent until at least 90 days after the last dose of study treatment regardless the start of new anticancer therapy. If Anti-GITR Antibody A or retifanlimab is permanently discontinued for toxicity, the combination drug must also be discontinued.

### 5. Study Eligibility

### 5.1 Inclusion Criteria

Subjects are eligible to be included in the study only if all of the following criteria apply:
1. Ability to comprehend and willingness to sign a written informed consent for the study.
2. Male or female subjects aged 18 years or older at the time of signing the informed consent.
3. Histologically or cytologically confirmed recurrent or metastatic HNSCC (e.g., oral cavity, oropharynx, hypopharynx, or larynx), that is not amenable to local therapy with curative intent (e.g., surgery or radiation therapy with or without chemotherapy).
   Subjects with primary carcinoma of the oropharynx should have documented HPV status (e.g., positive or negative) using local institutional testing method/assay (e.g., p16 IHC). Lack of local result does not prevent eligibility. HPV p16 status is not required for other eligible HNSCC primary tumor sites.
   Subjects with squamous cell carcinomas of the nasopharynx, salivary gland, or nonsquamous cell histology are excluded.
4. Subjects may have received prior adjuvant or neoadjuvant chemotherapy or chemotherapy as part of multimodal treatment for locally advanced disease. Systemic therapy must have been completed >6 months prior to enrollment, with no evidence of disease progression within 6 months of completion of systemic treatment.
5. Cohorts 1 and 2 (Part 1, lead-in) and Treatment Group A (Part 2, expansion):
   a. Have been previously treated with a PD-(L)1 inhibitor as monotherapy or in combination with systemic therapy for recurrent or metastatic HNSCC.
6. Treatment Group B (Part 2, expansion):
   a. Have not been previously treated with a PD-(L)1 inhibitor. May have received up to 1 prior line of chemotherapy for recurrent/metastatic HNSCC.
   b. PD-L1-positive tumor (defined by CPS≥1%) as per local laboratory determination.
7. Eastern Cooperation Oncology Group (ECOG) performance status of 0 to 1.
8. Measurable disease by CT or MRI based on RECIST v1.1 as determined by site radiology.

Tumor lesions located in a previously irradiated area, or in an area subjected to other locoregional therapy, are not considered measureable unless there has been documented radiographic progression in the lesion(s).
9. Baseline archival tumor specimen available or willingness to undergo a pretreatment tumor biopsy to obtain the specimen for biomarker analysis.
   Cohorts 1 and 2 (Part 1, lead-in) and Treatment Group A (Part 2, expansion): Availability of tissue from a core or excisional biopsy obtained after prior anti-PD-(L)1 therapy or willingness to undergo a screening biopsy before study initiation.
   AND, if available, archival tissue from a core or excisional biopsy obtained before prior anti-PD-(L)1 therapy
   Treatment Group B (Part 2, expansion):
   Availability of tissue from a core or excisional biopsy or willingness to undergo a screening biopsy before study initiation.
10. Have centrally confirmed GITR-positive tumor (defined as TPS≥10%) based on assessment of tumor biopsy tissue at a sponsor-designated laboratory before study treatment initiation.
11. Willingness to avoid pregnancy or fathering children based on the criteria below.
   a. Male subjects with reproductive potential must agree to take appropriate precautions to avoid fathering children from screening through 180 days after the last dose of study treatment and must refrain from donating sperm during this period.
   b. Female subjects who are WOCBP must have a negative pregnancy test at screening (serum test) and before the first dose on Day 1 (urine test) and must agree to take appropriate precautions to avoid pregnancy from screening through 180 days (after the last dose of study treatment) and must refrain from donating oocytes during this period.
   c. A female subject not considered to be of childbearing potential is eligible.

### 5.2 Exclusion Criteria

Subjects are excluded from the study only if any of the following criteria apply:
1. Have received anticancer medications or investigational drugs within the following intervals before the first administration of study treatment:
   a. ≤21 days for chemotherapy, targeted small molecule therapy, curative radiation therapy, and have recovered from acute radiation-induced toxicities.
      Palliative radiation therapy to single sites or small fields may be allowed with medical monitor approval. At least a 1-week washout period is permitted for palliative radiation to non-CNS disease with medical monitor approval.
      Subject must not have had radiation pneumonitis as a result of treatment.
   b. ≤28 days for a prior mAb used for anticancer therapy other than a PD-(L)1 inhibitor.
      Exception: denosumab may be used.
   c. y 28 days or 5 half-lives (whichever is longer) for all other investigational study drugs or devices. For investigational agents with long half-lives (e.g., >5 days), enrollment before the fifth half-life requires medical monitor approval.
2. Have received prior treatment with any TNFSF agonist (e.g., GITR, OX40, 4-1BB/CD137, CD27) for any indication.
3. Presence of tumors that invade major blood vessels, as shown unequivocally by imaging studies, and with active bleeding.
4. Have not recovered to Grade 1 or baseline from toxic effects of prior therapy (including prior immunotherapy) and/or complications from prior surgical intervention before starting study treatment.
   Subjects with stable chronic conditions (≤Grade 2) not expected to resolve (such as neuropathy or alopecia) are exceptions and may enroll.
5. Have laboratory values at screening defined in Table 7.

TABLE 7
Exclusionary Laboratory Values

| | |
|---|---|
| Laboratory | Exclusion |
| Parameter | Criterion |

Hematology

| | | |
|---|---|---|
| a | Platelets | <100 × 10⁹/L |
| b | Hemoglobin | <9 g/dL or <5.6 mmol/L |
| | | Note: transfusion is acceptable |
| | | to meet this criterion. |
| c | ANC | <1.0 × 10⁹/L |

Hepatic

| | | |
|---|---|---|
| d | ALT | ≥2.5 × ULN or ≥5 × ULN for |
| | | subjects with liver metastases. |
| e | AST | ≥2.5 × ULN or ≥5 × ULN for |
| | | subjects with liver metastases. |
| f | Total bilirubin | ≥1.5 × ULN OR >3.0 mg/dL for subjects |
| | | with documented Gilbert's syndrome. |

Renal

| | | |
|---|---|---|
| g | Creatinine | Calculated creatinine clearance |
| | clearance | <30 mL/min, according to |
| | | Cockroft-Gault formula. |

Coagulation

| | | |
|---|---|---|
| h | INR or PT | >1.5 × ULN, unless on therapeutic |
| | | anticoagulants and stable. |

ANC, absolute neutrophil count;
ALT, alanine transaminase;
AST, aspartate transaminase;
ULN, upper limit of normal;
INR, international normalized ratio;
PT, prothrombin time

6. Have known active CNS metastases/lymphoma and/or carcinomatous meningitis.
   Subjects who have previously treated and clinically stable brain or CNS metastases (without evidence of progression by imaging for at least 2 weeks before the first dose of study treatment and whose neurologic symptoms have returned to baseline), have no evidence of new or enlarging brain metastasis or CNS edema, and have not required steroids for at least 7 days before the first dose of study treatment are eligible.
7. Any of the following:
   a. Have known active HBV or HCV, defined as follows (testing must be performed to determine eligibility):
      Active HBV is defined as a known positive HBsAg result and positive total anti-HBc results.
      Active HCV is defined as a positive HCV antibody result and quantitative HCV-RNA results greater than the lower limits of detection of the assay.
   Subjects who have had definitive treatment for HCV are permitted if HCV-RNA is undetectable.
   Exception for subjects with serologic findings suggestive of HBV vaccination (anti-HBs positivity as the only serologic marker) AND a known history of prior HBV vaccination do not need to be tested for HBV DNA by PCR.
   b. Known to be seropositive for HIV. HIV testing is not required unless mandated by the local health authority.
8. Any known additional malignancy that is progressing or requires active treatment, or history of other malignancy within 3 years of the first dose of study treatment with the exception of cured basal cell or squamous cell carcinoma of the skin, superficial bladder cancer, prostate intraepithelial neoplasm, carcinoma in situ of the cervix, or other noninvasive or indolent malignancy, or cancers from which the participant has been disease-free for >1 year after treatment with curative intent.
9. Have an active autoimmune disease that has required systemic treatment in past 2 years (e.g., with use of disease modifying agents, corticosteroids, or immunosuppressive drugs). Replacement therapy (e.g., thyroxine, insulin, or physiologic corticosteroid replacement therapy for thyroid, adrenal, or pituitary insufficiency) is allowed.
10. Are on chronic systemic steroids (>10 mg/day of prednisone or equivalent).
   a. Physiologic corticosteroid replacement therapy at doses >10 mg/day of prednisone or equivalent for adrenal or pituitary insufficiency and in the absence of active autoimmune disease is permitted.
   b. Subjects with a condition (e.g., asthma or COPD) that requires intermittent use of bronchodilators, inhaled steroids, or local steroid injections may participate.
   c. Subjects using topical, ocular, intra-articular, or intranasal steroids (with minimal systemic absorption) may participate.
   d. Brief courses of corticosteroids for prophylaxis (e.g., contrast dye allergy) or study treatment-related standard premedication are permitted.
11. Active infections requiring systemic antibiotics or antifungal or antiviral treatment (within 14 days before first dose of study treatment).
   Subjects should be excluded if they have a positive screening test result for SARS-CoV2 infection, until test normalization and clinical recovery.
12. Current use of any prohibited medication as described in Section 6.
13. Evidence of interstitial lung disease or history of interstitial lung disease, or active, noninfectious pneumonitis.
14. History of organ transplant, including allogeneic stem cell transplantation.
15. Known hypersensitivity to any of the study drugs, excipients, or another mAb that cannot be controlled with standard measures (e.g., antihistamines and corticosteroids).
16. History or presence of an abnormal ECG that, in the investigator's opinion, is clinically meaningful. Screening QTcF interval >470 milliseconds is excluded. In the event that a single QTcF is >470 milliseconds, the screening ECG may be repeated in triplicate, and the participant may enroll if the average QTcF is ≤470 milliseconds.
17. Have had a significant cardiac event within 6 months before the first dose of study treatment, including New York Heart Association Class IIIV, acute myocardial infarction (including severe/unstable angina), cardiomyopathy, coronary/peripheral artery bypass graft, symptomatic congestive heart failure, critical conduction delay, cerebrovascular accident or transient ischemic attack, or pulmonary embolism.
   Medically-controlled arrhythmia is permitted.
   Recovery from uncontrolled or Grade ≥3 hypertension to ≤Grade 1 is required.
18. Have received of a live vaccine within 30 days of planned start of study treatment.
   Examples of live vaccines include but are not limited to the following: measles, mumps, rubella, chicken pox/zoster, yellow fever, rabies, Bacillus Calmette-Guerin, and typhoid vaccine. Seasonal influenza vaccines for injection are generally killed virus vaccines and are allowed; however, intranasal influenza vaccines are live-attenuated vaccines and are not allowed.
   SARS-CoV2 vaccine is allowed as long as it is not a live vaccine.
19. Known or suspected SARS-CoV-2 infection at the time of enrollment.
   Subjects with a positive test result for SARS-CoV-2 infection at any time during screening should not be enrolled until a negative PCR test and clinical recovery (as applicable) are confirmed.
20. Concurrent anticancer therapy (e.g., chemotherapy, radiation therapy, surgery, immunotherapy, biologic therapy, hormonal therapy, investigational therapy, or tumor embolization).
21. Women who are pregnant or breastfeeding.

22. Any condition that would, in the investigator's judgment, interfere with full participation in the study, including administration of study treatment and attending required study visits; pose a significant risk to the participant; or interfere with interpretation of study data.

### Example 2: A Study of an Anti-GITR Antibody in Combination with Immune Therapies in Subjects with Selected Advanced Malignancies

This is a Phase 1/2, open-label, multicenter study of an anti-GITR antibody (Anti-GITR Antibody A, as described in Table 1) in combination with an anti-PD-1 antibody (nivolumab, as described in Table 2) and/or an anti-CTLA-4 antibody (ipilimumab, as described in Table 4) in subjects with selected advanced malignancies.

Part 1 of the study is a dose escalation study in participants with selected advanced malignancies. Part 1 will evaluate the safety, tolerability, and dose-limiting toxicities (DLTs) of Anti-GITR Antibody A in combination with immune therapies and to define the recommended Phase 2 dose(s) of Anti-GITR Antibody A when given in combination with immune therapies. The select advanced malignancies included in Part 1 are cervical cancer, endometrial cancer, gastric cancer (including stomach, esophageal, and gastroesophageal junction (GEJ) cancer), hepatocellular carcinoma (HCC), melanoma, Merkel cell carcinoma, mesothelioma, microsatellite instability-high (MSI-H) colorectal cancer (CRC), non-small cell lung cancer (NSCLC), ovarian cancer, squamous cell carcinoma of the head and neck (SCCHN), small cell lung cancer (SCLC), renal cell carcinoma (RCC), triple-negative breast cancer (TNBC), and urothelial carcinoma.

Part 2 of the study will further evaluate the safety, tolerability, efficacy, pharmacokinetic and pharmacologic activity of the immune therapy combinations in subjects with advanced or metastatic cervical cancer, gastric cancer (including stomach, esophageal, and GEJ), SCCHN, and PD-1 or PD-L1 relapsed melanoma. Additional tumor-specific cohorts may be added.

### Justification for Dose

The proposed starting dose and schedule of Anti-GITR Antibody A (0.1 mg/kg IV Q2W) is based on preclinical data and emerging clinical data from the ongoing first in human monotherapy study.

To ensure safety in combination with nivolumab and ipilimumab in this study, the starting dose of Anti-GITR Antibody A will be defined as 1 dose level below the highest tolerated dose of Anti-GITR Antibody A when given as monotherapy. Current clinical observations from the ongoing monotherapy study have shown safety and tolerability of Anti-GITR Antibody A when administered at a dose up to 0.3 mg/kg IV Q2W. As data emerges from study the ongoing monotherapy study, a higher starting dose of Anti-GITR Antibody A may be used but will not exceed 1 dose level below the highest tolerated monotherapy dose of Anti-GITR Antibody A.

The current schedule of administration (Q2W) is based on both preclinical and clinical observations. The Q2W schedule used in the ongoing monotherapy study was based on the PK of Anti-GITR Antibody A determined in African green monkeys (AGMs) following a single and 4 multiple (weekly) IV doses. In both single and repeated dose administrations, the disposition of Anti-GITR Antibody A was biphasic, with mean plasma half-life values ranging from 4 to 12 days. These preclinical findings support Q2W dosing and are consistent with observations in the first-in-human ongoing monotherapy study.

The approved dose of nivolumab (240 mg Q2W) has been selected for the doublet combination (nivolumab and Anti-GITR Antibody A). The approved doses of nivolumab and ipilimumab in combination are nivolumab (3 mg/kg Q2W) and ipilimumab (3 mg/kg Q3W for 4 doses). Additional combination studies of nivolumab (3 mg/kg Q2W) and ipilimumab (1 mg/kg Q6W) showed similar efficacy with better tolerability. To minimize any additional risk to safety, Anti-GITR Antibody A will be combined with nivolumab (3 mg/kg Q2W) and ipilimumab (1 mg/kg Q6W) in the triplet cohorts (nivolumab, ipilimumab, and Anti-GITR Antibody A).

Fixed dosing has several advantages over weight-based dosing, including convenience of preparation and administration, reducing errors in preparation calculation, and minimization of drug waste. Body size-based dosing and fixed dosing of monoclonal antibodies have been evaluated, with the 2 dosing approaches performing similarly, and with body size-based dosing not always offering an advantage in reducing variability of exposure. These studies concluded that either approach may be used, and that fixed dosing is recommended as the preferred approach because of the advantages mentioned above.

Enrollment in the Phase 2 portion of the study will begin when the maximum tolerated dose (MTD) or pharmacologically active dose (PAD) of Anti-GITR Antibody A for a given treatment group in Phase 1 has been determined. The RP2D of Anti-GITR Antibody A in a combination must be based on safety data from Phase 1 (dose escalation in combination), as well as safety data from the monotherapy study. In addition to the safety profile, pharmacologic analyses from the monotherapy study have been used to define the RP2D of Anti-GITR Antibody A as a monotherapy. Receptor occupancy saturation has been observed at doses as low as 0.3 mg/kg Q2W. However, the detection of a neutralizing ADA of Anti-GITR Antibody A has been observed at a frequency and intensity inversely proportional to the dose of Anti-GITR Antibody A. The ADA has only been observed at doses ≤1.0 mg/kg Q2W. ADA has not been observed at doses ≥3 mg/kg Q2W. When given in combination with nivolumab, ADA has also been observed at doses ≤1.0 mg/kg Q2W but not at doses ≥3 mg/kg Q2W. Taken together, this would suggest that although 0.3 mg/kg would sufficiently saturate GITR receptors, it would be prudent to use a high enough dose that minimizes the risk of developing an ADA. The use of PK modeling and simulations helped determine the best flat doses (given every 2, 3, or 4 weeks) that would avoid trough levels associated with ADA. Administering between 3 and 5 mg/kg Q2W would insure a dose that consistently provides saturated receptors and adequately avoids ADA.

The current RP2D for monotherapy Anti-GITR Antibody A is 300 mg (˜4 mg/kg) Q2W or Q3W. Therefore, once the MTD is determined for a given treatment group in Phase 1, the RP2D may be converted to a fixed dose.

### Doublet Immune Therapy Combinations

Phase 1 will begin with 3 doublet treatment groups, as outlined in Table 8, which will is be explored in parallel.

TABLE 8
Doublet Immune Therapy Treatment Groups

| | | |
|---|---|---|
| Treatment | Anti-GITR Antibody A | Nivolumab |
| Group A | Concurrent Dosing | |
| | 1.0 mg/kg, 3.0 mg/kg, 5.0 mg/kg, or | 240 mg Q2W |
| | 10.0 mg/kg Q2W | |
| Treatment | Anti-GITR Antibody A | Nivolumab |
| Group B | Sequenced Dosing | |
| | 1.0 mg/kg, 3.0 mg/kg, 5.0 mg/kg, or | 240 mg Q2W starting |
| | 10.0 mg/kg Q2W | at Cycle 3 |
| | Run-in with Anti-GITR Antibody A | |
| | Q2W for 2 doses | |
| Treatment | Anti-GITR Antibody A | Ipilimumab |
| Group C | Concurrent Dosing | |
| | 1.0 mg/kg, 3.0 mg/kg, or 5.0 mg/kg | 1 mg/kg Q6W |
| | Q2W | |

### Triplet Immune Therapy Combinations

Dose escalation of the triplet immune therapy combinations will begin enrolling once all of the applicable doublet combinations have cleared 3 Anti-GITR Antibody A dose levels or the MTD or PAD of Anti-GITR Antibody A has been determined (whichever occurs first). The starting dose of Anti-GITR Antibody A will be 2 dose levels below the last dose cohort deemed safe in the doublet combination. For example, if 3 mg/kg of Anti-GITR Antibody A is safe in the doublet combinations with both nivolumab and ipilimumab, then the starting dose in the triplet will be 0.3 mg/kg. If the MTD of Anti-GITR Antibody A is 1 mg/kg in the doublet combinations, then the starting dose of Anti-GITR Antibody A for the triplet immune therapy combination will be 0.1 mg/kg. If there are different MTDs of Anti-GITR Antibody A with nivolumab and ipilimumab, then the starting dose of the triplet will be 2 dose levels below the lowest MTD in the doublet. The triplet immune therapy combinations will be explored in parallel as outlined in Table 9.

TABLE 9
Triplet Immune Therapy Combinations

| | | | |
|---|---|---|---|
| Treatment | Anti-GITR Antibody A | Nivolumab | Ipilimumab |
| Group D | Concurrent Dosing | | |
| | 1.0 mg/kg Q2W | 3 mg/kg Q2W | 1 mg/kg Q6W |
| Treatment | Anti-GITR Antibody A | Nivolumab | Ipilimumab |
| Group E | Sequenced Followed by | | |
| | Concurrent Dosing | | |
| | 0.1 mg/kg (starting dose), | 240 mg Q2W | 1 mg/kg Q6W |
| | 0.3 mg/kg, 1.0 mg/kg, 3.0 | starting at | starting at |
| | mg/kg, 5.0 mg/kg, or 10.0 | Cycle 3 | Cycle 3 |
| | mg/kg Q2W | | |
| | Run-in with Anti-GITR | | |
| | Antibody A Q2W for 2 | | |
| | doses | | |

### Phase 2 Dose Expansion

Phase 2 of the study will further evaluate the safety, tolerability, efficacy, PK, and pharmacologic activity of the immune therapy combinations in subjects with advanced or metastatic cervical cancer, gastric cancer (including stomach, esophageal, and GEJ), SCCHN, PD-1 refractory SCCHN, and PD-1 or PD-L1 relapsed melanoma. Additional tumor-specific cohorts may be added, by protocol amendment, based on emerging data. Alternate dose administration schedules and/or fixed doses of Anti-GITR Antibody A (comparable to or less than the highest dose levels determined to be safe or pharmacologically active) may also be explored depending on PK, translational biomarkers, and safety results.

The Phase 2 expansion treatment groups and tumor-specific cohorts for each treatment group are outlined in Table 10.

TABLE 10
Phase 2 Expansion Treatment Groups
Doublet Expansion Treatment Groups

| | | | |
|---|---|---|---|
| Treatment | Anti-GITR Antibody A | Ipilimumab | Tumor Cohorts |
| Group C2 | Concurrent Dosing | | |
| | RP2D of Anti-GITR | 1 mg/kg Q6W | Cohort 1 - PD- |
| | Antibody A Q2W | | 1/PD-L1 relapsed |
| | | | melanoma |
| | | | Cohort 2- Biopsy |
| Treatment | Anti-GITR Antibody A | Nivolumab | Tumor Cohorts |
| Group F | Concurrent Dosing | | |
| | RP2D of Anti-GITR | 240 mg Q2W | Cohort 1 - Cervical |
| | Antibody A Q2W | | Cohort 2 - Gastric |
| | | | Cohort 3 - SCCHN |
| | | | Cohort 4 - PD- |
| | | | 1/PD-L1 relapsed |
| | | | melanoma |
| | | | Cohort 5 - Biopsy |
| | | | Cohort 6 - PD-1 |
| | | | refractory SCCHN |
| Treatment | Anti-GITR Antibody A | Nivolumab | Tumor Cohorts |
| Group H | Sequenced Followed by | | |
| | Concurrent Dosing | | |
| | RP2D of Anti-GITR | 240 mg Q2W starting at Cycle 3 in | Cohort 1 - Cervical |
| | Antibody A run-in Q2W for | combination with Anti-GITR Antibody | Cohort 2 - Gastric |
| | 2 doses followed by Anti- | A Q2W | Cohort 3 - SCCHN |
| | GITR Antibody A Q2W | | Cohort 4 - Biopsy |

Triplet Expansion Treatment Groups

| | | | | |
|---|---|---|---|---|
| Treatment | Anti-GITR Antibody A | Nivolumab | Ipilimumab | Tumor Cohorts |
| Group J | Concurrent Dosing | | | |
| | RP2D of Anti-GITR | 3 mg/kg Q2W | 1 mg/kg Q6W | Cohort 1 - Cervical |
| | Antibody A Q2W | | | Cohort 2 - Gastric |
| | | | | Cohort 3 - SCCHN |
| | | | | Cohort 4 - Biopsy |
| Treatment | Anti-GITR Antibody A | Nivolumab | Ipilimumab | Tumor Cohorts |
| Group L | Sequenced Followed by | | | |
| | Concurrent Dosing | | | |
| | RP2D of Anti-GITR | 3 mg/kg Q2W | 1 mg/kg Q6W | Cohort 1 - Cervical |
| | Antibody A run-in Q2W for | starting at Cycle | starting at Cycle 3 | Cohort 2 - Gastric |
| | 2 doses followed by Anti- | 3 | | Cohort 3 - SCCHN |
| | GITR Antibody A Q2W | | | Cohort 4 - Biopsy |

### Anti-GITR Antibody A Regimen

The starting dose of ANTIBODY A in Part 1 is 0.1 mg/kg administered by intravenous infusion Q2W. The following additional dose levels will be evaluated during Part 1 of the study: 0.3 mg/kg, 1.0 mg/kg, 3.0 mg/kg, 5.0 mg/kg, and 10.0 mg/kg.

### Nivolumab Regimen

Nivolumab will be administered by intravenous infusion at a dose that is dependent on the assigned treatment group:
Doublet Treatment Group (Anti-GITR Antibody A and nivolumab): 240 mg on Day 1 of every 2-week cycle.
Triplet Treatment Group (Anti-GITR Antibody A, nivolumab, and ipilimumab): 3 mg/kg on Day 1 of every 2-week cycle.
Concurrent dosing Treatment Groups: Nivolumab dosing will begin on Cycle 1 Day 1.
Sequenced dosing Treatment Groups: Nivolumab dosing will begin on Cycle 3 Day 1.

Nivolumab will be administered at least 30 minutes after the infusion of Anti-GITR Antibody A (when applicable), and subjects will continue to receive nivolumab as long as the subject is deriving benefit and has not met any of the conditions for treatment withdrawal, or for up to 24 months from the first dose of study treatment, whichever occurs first.

### Ipilimumab Regimen

Ipilimumab will be administered by intravenous infusion at a dose that is dependent on the assigned treatment group:
Doublet Treatment Group (Anti-GITR Antibody A and ipilimumab): 1 mg/kg on Day 1 of every 6-week cycle.
Triplet Treatment Group (Anti-GITR Antibody A, nivolumab, and ipilimumab): 1 mg/kg on Day 1 of every 6-week cycle.
Concurrent dosing Treatment Groups: Ipilimumab dosing will begin on Cycle 1 Day 1.
Sequenced dosing Treatment Groups: Ipilimumab dosing will begin on Cycle 3 Day 1.

Ipilimumab will be administered at least 30 minutes after the infusion of Anti-GITR Antibody A (when administered on the same day). Ipilimumab will be administered after Anti-GITR Antibody A and nivolumab. Subjects will continue to receive nivolumab as long as the subject is deriving benefit and has not met any of the conditions for treatment withdrawal, or for up to 24 months from the first dose of study treatment, whichever occurs first.

Once the MTD or PAD of Anti-GITR Antibody A has been established with ipilimumab at 1 mg/kg Q6W, a higher dose of ipilimumab at 3 mg/kg Q6W and/or 3 mg/kg Q3W for 4 doses may be tested.

### Objectives and Endpoints

The primary objective of the Phase 1 study is to evaluate the safety, tolerability, and dose-limiting toxicities (DLTs) of Anti-GITR Antibody A in combination with immune therapies and to define the RP2D(s) of Anti-GITR Antibody A when given in combination with immune therapies.

The primary objective of the Phase 2 study is to evaluate the efficacy of Anti-GITR Antibody A when given in combination with immune therapies by assessing objective response rate (ORR).

The secondary objectives of the Phase 1 and Phase 2 study are: (1) to determine the efficacy of Anti-GITR Antibody A in terms of objective response rate (ORR), disease control rate (DCR), duration of response (DOR), duration of disease control, and progression-free survival (PFS) in participants with selected advanced malignancies; (2) to evaluate the efficacy of Anti-GITR Antibody A when given in combination with immune therapies with respect to 1-year and 2-year overall survival; and (3) to evaluate the safety and tolerability of Anti-GITR Antibody A when given in combination with immune therapies.

The primary objectives are evaluated by measuring the following endpoints: (1) frequency, duration, and severity of AEs, and (2) ORR defined as the percentage of subjects having complete response (CR) or partial response (PR).

The secondary objectives are evaluated by measuring the following endpoints: (1) ORR defined as the percentage of subjects having complete response (CR) or partial response (PR); (2) DOR, defined as the time from the earliest date of disease response (CR or PR) until earliest date of disease progression or death due to any cause, if occurring sooner than progression; (3) DCR, defined as the percentage of subjects having CR, PR, or SD; (4) duration of disease control (CR, PR, and SD) as measured from first report of SD or better until disease progression or death from any cause, if occurring sooner than progression, (5) PFS, defined as the time from the start of combination therapy until the earliest date of disease progression or death due to any cause, if occurring sooner than progression, (6) OS determined from the start of combination therapy until death due to any cause; and (7) safety and tolerability will be assessed by monitoring frequency, duration, and severity of AEs.

### Results

A total of 51 patients (Treatment Group A, n=17; Treatment Group B, n=13; Treatment Group C, n=15; Treatment Group D, n=6) were enrolled (most common tumors: breast, gastric, and ovarian [each n=8]; prior PD-(L)1 therapy, n=12). One patient in Treatment Group D had DLTs (nonserious grade 3 pruritis and rash); MTD was not reached; Anti-GITR Antibody A 300 mg Q2W was selected as RP2D. All patients in Treatment Groups A, B, and D, had treatment-emergent adverse events (TEAEs), and 93.3% of patients in Treatment Group C had TEAEs. Twenty-six patients had grade ≥3 TEAEs (Treatment Group A, n=10; Treatment Group B, n=5; Treatment Group C, n=7; Treatment Group D, n=4), most commonly, pneumothorax (Treatment Group A [5 mg/kg], n=2) and anemia (Treatment Group B [1, 5 mg/kg], n=2; Treatment Group C [1, 3, 5 mg/kg], n=3). Anti-GITR Antibody A-related TEAEs occurred in 8, 6, 10, and 5 pts in Treatment Groups A, B, C, and D, respectively. Most common immune-related AEs in Treatment Group A: pruritus (grade 1/2, n=2); in Treatment Group B: rash (grade 1, n=2), simultaneous rash and pruritus (grade 1, n=4); in Treatment Group C: pruritus (grade 1/2, n=2), rash (grade 1, n=2), simultaneous rash and pruritus (grade 1, n=2); in Treatment Group D: simultaneous pruritus and rash (grade 3, n=1); 3 pts (Treatment Group A [1 mg/kg], n=1; Treatment Group C [3 mg/kg], n=1; Treatment Group D, n=1) had infusion-related reactions. One patient in Treatment Group A [10 mg/kg Q2W] discontinued treatment due to a pneumonia TEAE. Best unconfirmed overall responses (per RECIST v1.1) were: 1 complete response (CR; duration of response [DOR], 1065 days) in renal cancer and 1 partial response (PR; DOR, 573 days) in breast cancer in Treatment Group A; 1 CR (DOR, 876 days) in cervical cancer in Treatment Group B; 1 PR (DOR, 169 days) in ovarian cancer and 1 PR (DOR, 281 days) in lung cancer in Treatment Group C; none in Treatment Group D. The disease control rate was 35.3% in Treatment Group A, 23.1% in Treatment Group B, 33.3% in Treatment Group C, and 0% in Treatment Group D.

Anti-GITR Antibody A plus nivolumab and/or ipilimumab was generally well tolerated in patients with advanced tumors; most common toxicities were mild to moderate pruritus and rash. No novel safety signals were seen. Anti-GITR Antibody A 300 mg Q2W was selected as RP2D based on safety and preliminary PK/PD data and used for expansion in combination with nivolumab and/or ipilimumab.

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method for treating a cancer in a human subject in need thereof, the method comprising administering to the human subject an effective amount of an anti-GITR antibody and an anti-PD-1 antibody, wherein the anti-GITR antibody comprises a heavy chain variable region (VH) comprising a VH CDR1, a VH CDR2, and a VH CDR3, and a light chain variable region (VL) comprising a VL CDR1, a VL CDR2, and a VL CDR3, wherein the VH CDR1 comprises the amino acid sequence of DYAMY (SEQ ID NO:1), the VH CDR2 comprises the amino acid sequence of VIRTYSGDVTYNQKFKD (SEQ ID NO:2), the VH CDR3 comprises the amino acid sequence of SGTVRGFAY (SEQ ID NO:3), the VL CDR1 comprises the amino acid sequence of KSSQSLLNSGNQKNYLT (SEQ ID NO:4), the VL CDR2 comprises the amino acid sequence of WASTRES (SEQ ID NO: 5), and the VL CDR3 comprises the amino acid sequence of QNDYSYPYT (SEQ ID NO:6).

2. The method of claim 1, wherein the method does not comprise administration of a multivalent autophagosome cancer vaccine to the human subject.

3. The method of claim 1, wherein the method does not comprise administration of a cancer vaccine to the human subject.

4. The method of claim 1, wherein the human subject has an advanced solid tumor, advanced renal cell carcinoma, anal cancer, bladder cancer, brain cancer, breast cancer, cervical cancer, colorectal cancer, endometrial cancer, esophageal cancer, gastric cancer, gastrointestinal cancer, gastroesophageal cancer, glioblastoma, head and neck squamous cell carcinoma (HNSCC), hepatocellular carcinoma (HCC), glioma, kidney cancer, liver cancer, lung cancer, lymphoma, melanoma, Merkel cell carcinoma, mesothelioma, multiple myeloma, myeloma, neuroblastoma, non-small cell lung cancer (NSCLC), ovarian cancer, pancreatic cancer, prostate cancer, rectal cancer, renal cell carcinoma (RCC), sarcoma, skin cancer, small cell lung cancer (SCLC), soft-tissue sarcoma, thoracic cancer, thyroid cancer, triple-negative breast cancer (TNBC), urothelial cancer, uterine cancer, or a combination thereof

5.-8. (canceled)

9. The method of claim 7, wherein HNSCC tumor cells in a biological sample obtained from the human subject express GITR.

10. The method of claim 9, comprising determining that HNSCC tumor cells in a biological sample obtained from the human subject express GITR.

11. The method of claim 9, wherein at least 10% of HNSCC tumor cells in the biological sample obtained from the human subject express GITR.

12. The method of claim 1, wherein the human subject has undergone a prior anti-cancer therapy.

13-14. (canceled)

15. The method of claim 1, wherein the human subject has progressed on or after prior systemic treatment.

16. (canceled)

17. The method of claim 1, wherein the anti-GITR antibody is administered to the human subject intravenously at a dose between 0.1 to 1000 mg.

18.-19. (canceled)

20. The method of claim 1, wherein the anti-GITR antibody is administered to the human subject intravenously at a dose between 0.3 mg/kg to 10 mg/kg.

21.-27. (canceled)

28. The method of claim 1, wherein the anti-GITR antibody and the anti-PD-1 antibody are administered simultaneously or sequentially.

29. The method of claim 1, wherein the anti-GITR antibody is administered to the human subject once every two weeks.

30.-31. (canceled)

32. The method of claim 1, wherein the anti-PD-1 antibody is administered to the human subject once every two weeks.

33.-65. (canceled)

66. The method of claim 1, wherein the anti-PD-1 antibody comprises a VH comprising a VH CDR1, a VH CDR2, and a VH CDR3, and a VL comprising a VL CDR1, a VL CDR2, and a VL CDR3, wherein the VH CDR1 comprises the amino acid sequence SYWMN (SEQ ID NO:11), the VH CDR2 comprises the amino acid sequence VIIIPSDSETWLDQKFKD (SEQ ID NO:12), the VH CDR3 comprises the amino acid sequence EHYGTSPFAY (SEQ ID NO:13), the VL CDR1 comprises the amino acid sequence RASESVDNYGMSFMNW (SEQ ID NO:14), the VL CDR2 comprises the amino acid sequence AASNQGS (SEQ ID NO:15), and the VL CDR3 comprises the amino acid sequence QQSKEVPYT (SEQ ID NO:16).

67.-70. (canceled)

71. The method of claim 1, wherein the anti-PD-1 antibody comprises a VH comprising a VH CDR1, a VH CDR2, and a VH CDR3, and a VL comprising a VL CDR1, a VL CDR2, and a VL CDR3, wherein the VH CDR1 comprises the amino acid sequence NYYMY (SEQ ID NO:21), the VH CDR2 comprises the amino acid sequence GINPSNGGTNFNEKFKN (SEQ ID NO:22), the VH CDR3 comprises the amino acid sequence RDYRFDMGFDY (SEQ ID NO:23), the VL CDR1 comprises the amino acid sequence RASKGVSTSGYSYLH (SEQ ID NO:24), the VL CDR2 comprises the amino acid sequence LASYLES (SEQ ID NO:25), and the VL CDR3 comprises the amino acid sequence QHSRDLPLT (SEQ ID NO:26).

72.-75. (canceled)

76. The method of claim 1, wherein the anti-PD-1 antibody comprises a VH comprising a VH CDR1, a VH CDR2, and a VH CDR3, and a VL comprising a VL CDR1, a VL CDR2, and a VL CDR3, wherein the VH CDR1 comprises the amino acid sequence NSGMH (SEQ ID NO:31), the VH CDR2 comprises the amino acid sequence VIWYDGSKRYYADSVKG (SEQ ID NO:32), the VH CDR3 comprises the amino acid sequence NDDY (SEQ ID NO:33), the VL CDR1 comprises the amino acid sequence RASQSVSSYLA (SEQ ID NO:34), the VL CDR2 comprises the amino acid sequence DASNRAT (SEQ ID NO:35), and the VL CDR3 comprises the amino acid sequence QQSSNWPRT (SEQ ID NO:36).

77.-80. (canceled)

81. The method of claim 1, wherein the anti-PD-1 antibody comprises a VH comprising a VH CDR1, a VH CDR2, and a VH CDR3, and a VL comprising a VL CDR1, a VL CDR2, and a VL CDR3, wherein the VH CDR1 comprises the amino acid sequence NSGMH (SEQ ID NO:41), the VH CDR2 comprises the amino acid sequence VIWYDGSKRYYADSVKG (SEQ ID NO:42), the VH CDR3 comprises the amino acid sequence NDDY (SEQ ID NO:43), the VL CDR1 comprises the amino acid sequence RASQSVSSYLA (SEQ ID NO:44), the VL CDR2 comprises the amino acid sequence DASNRAT (SEQ ID NO:45), and the VL CDR3 comprises the amino acid sequence QQSSNWPRT (SEQ ID NO:46).

82.-104. (canceled)

105. A method for treating a cancer in a human subject in need thereof, the method comprising administering to the human subject an effective amount of an anti-GITR antibody and an anti-PD-L1 antibody, wherein the anti-GITR antibody comprises a heavy chain variable region (VH) comprising a VH CDR1, a VH CDR2, and a VH CDR3, and a light chain variable region (VL) comprising a VL CDR1, a VL CDR2, and a VL CDR3, wherein the VH CDR1 comprises the amino acid sequence of DYAMY (SEQ ID NO:1), the VH CDR2 comprises the amino acid sequence of VIRTYSGDVTYNQKFKD (SEQ ID NO:2), the VH CDR3 comprises the amino acid sequence of SGTVRGFAY (SEQ ID NO:3), the VL CDR1 comprises the amino acid sequence of KSSQSLLNSGNQKNYLT (SEQ ID NO:4), the VL CDR2 comprises the amino acid sequence of WASTRES (SEQ ID NO: 5), and the VL CDR3 comprises the amino acid sequence of QNDYSYPYT (SEQ ID NO:6).

106. The method of claim 105, wherein the human subject has an advanced solid tumor, advanced renal cell carcinoma, anal cancer, bladder cancer, brain cancer, breast cancer, cervical cancer, colorectal cancer, endometrial cancer, esophageal cancer, gastric cancer, gastrointestinal cancer, gastroesophageal cancer, glioblastoma, head and neck squamous cell carcinoma (HNSCC), hepatocellular carcinoma (HCC), glioma, kidney cancer, liver cancer, lung cancer, lymphoma, melanoma, Merkel cell carcinoma, mesothelioma, multiple myeloma, myeloma, neuroblastoma, non-small cell lung cancer (NSCLC), ovarian cancer, pancreatic cancer, prostate cancer, rectal cancer, renal cell carcinoma (RCC), sarcoma, skin cancer, small cell lung cancer (SCLC), soft-tissue sarcoma, thoracic cancer, thyroid cancer, triple-negative breast cancer (TNBC), urothelial cancer, uterine cancer, or a combination thereof.

107.-112. (canceled)

113. A method for treating a cancer in a human subject in need thereof, the method comprising administering to the human subject an effective amount of an anti-GITR antibody and an anti-CTLA-4 antibody, wherein the anti-GITR antibody comprises a heavy chain variable region (VH) comprising a VH CDR1, a VH CDR2, and a VH CDR3, and a light chain variable region (VL) comprising a VL CDR1, a VL CDR2, and a VL CDR3, wherein the VH CDR1 comprises the amino acid sequence of DYAMY (SEQ ID NO:1), the VH CDR2 comprises the amino acid sequence of VIRTYSGDVTYNQKFKD (SEQ ID NO:2), the VH CDR3 comprises the amino acid sequence of SGTVRGFAY (SEQ ID NO:3), the VL CDR1 comprises the amino acid sequence of KSSQSLLNSGNQKNYLT (SEQ ID NO:4), the VL CDR2 comprises the amino acid sequence of WASTRES (SEQ ID NO: 5), and the VL CDR3 comprises the amino acid sequence of QNDYSYPYT (SEQ ID NO:6).

114. The method of claim 113, wherein the method does not comprise administration of a multivalent autophagosome cancer vaccine to the human subject.

115. The method of claim 113, wherein the method does not comprise administration of a cancer vaccine to the human subject.

116. The method of claim 113, wherein the human subject has an advanced solid tumor, advanced renal cell carcinoma, anal cancer, bladder cancer, brain cancer, breast cancer, cervical cancer, colorectal cancer, endometrial cancer, esophageal cancer, gastric cancer, gastrointestinal cancer, gastroesophageal cancer, glioblastoma, head and neck squamous cell carcinoma (HNSCC), hepatocellular carcinoma (HCC), glioma, kidney cancer, liver cancer, lung cancer, lymphoma, melanoma, Merkel cell carcinoma, mesothelioma, multiple myeloma, myeloma, neuroblastoma, non-small cell lung cancer (NSCLC), ovarian cancer, pancreatic cancer, prostate cancer, rectal cancer, renal cell carcinoma (RCC), sarcoma, skin cancer, small cell lung cancer (SCLC), soft-tissue sarcoma, thoracic cancer, thyroid cancer, triple-negative breast cancer (TNBC), urothelial cancer, uterine cancer, or a combination thereof.

117.-198. (canceled)


SEQUENCE LISTING (110 sequences)

SEQ ID NO: 1 - protein, 5 aa, synthetic construct
DYAMY

SEQ ID NO: 2 - protein, 17 aa, synthetic construct
VIRTYSGDVTYNQKFKD

SEQ ID NO: 3 - protein, 9 aa, synthetic construct
SGTVRGFAY

SEQ ID NO: 4 - protein, 17 aa, synthetic construct
KSSQSLLNSGNQKNYLT

SEQ ID NO: 5 - protein, 7 aa, synthetic construct
WASTRES

SEQ ID NO: 6 - protein, 9 aa, synthetic construct
QNDYSYPYT

SEQ ID NO: 7 - protein, 118 aa, synthetic construct
QVQLVQSGAEVKKPGASVKVSCKGSGYTFTDYAMYWVRQAPGQGLEWIGVIRTYSGDVTYNQKFKDRATMTVDKSISTAYMELSRLRSDDTAVYYCAKSGTVRGFAYWGQGTLVTVSS

SEQ ID NO: 8 - protein, 113 aa, synthetic construct
DIVMTQSPDSLAVSLGERATINCKSSQSLLNSGNQKNYLTWYQQKPGQPPKLLIYWASTRESGVPDRFSGSGSGTDFTLTISSLQAEDVAVYHCQNDYSYPYTFGQGTKLEIK

SEQ ID NO: 9 - protein, 447 aa, synthetic construct
QVQLVQSGAEVKKPGASVKVSCKGSGYTFTDYAMYWVRQAPGQGLEWIGVIRTYSGDVTYNQKFKDRATMTVDKSISTAYMELSRLRSDDTAVYYCAKSGTVRGFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 10 - protein, 220 aa, synthetic construct
DIVMTQSPDSLAVSLGERATINCKSSQSLLNSGNQKNYLTWYQQKPGQPPKLLIYWASTRESGVPDRFSGSGSGTDFTLTISSLQAEDVAVYHCQNDYSYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 5 aa, synthetic construct
SYWMN

SEQ ID NO: 12 - protein, 17 aa, synthetic construct
VIHPSDSETWLDQKFKD

SEQ ID NO: 13 - protein, 10 aa, synthetic construct
EHYGTSPFAY

SEQ ID NO: 14 - protein, 16 aa, synthetic construct
RASESVDNYGMSFMNW

SEQ ID NO: 15 - protein, 7 aa, synthetic construct
AASNQGS

SEQ ID NO: 16 - protein, 9 aa, synthetic construct
QQSKEVPYT

SEQ ID NO: 17 - protein, 119 aa, synthetic construct
QVQLVQSGAEVKKPGASVKVSCKASGYSFTSYWMNWVRQAPGQGLEWIGVIHPSDSETWLDQKFKDRVTITVDKSTSTAYMELSSLRSEDTAVYYCAREHYGTSPFAYWGQGTLVTVSS

SEQ ID NO: 18 - protein, 111 aa, synthetic construct
EIVLTQSPATLSLSPGERATLSCRASESVDNYGMSFMNWFQQKPGQPPKLLIHAASNQGSGVPSRFSGSGSGTDFTLTISSLEPEDFAVYFCQQSKEVPYTFGGGTKVEIK

SEQ ID NO: 19 - protein, 445 aa, synthetic construct
QVQLVQSGAEVKKPGASVKVSCKASGYSFTSYWMNWVRQAPGQGLEWIGVIHPSDSETWLDQKFKDRVTITVDKSTSTAYMELSSLRSEDTAVYYCAREHYGTSPFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 20 - protein, 218 aa, synthetic construct
EIVLTQSPATLSLSPGERATLSCRASESVDNYGMSFMNWFQQKPGQPPKLLIHAASNQGSGVPSRFSGSGSGTDFTLTISSLEPEDFAVYFCQQSKEVPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 21 - protein, 5 aa, synthetic construct
NYYMY

SEQ ID NO: 22 - protein, 17 aa, synthetic construct
GINPSNGGTNFNEKFKN

SEQ ID NO: 23 - protein, 11 aa, synthetic construct
RDYRFDMGFDY

SEQ ID NO: 24 - protein, 15 aa, synthetic construct
RASKGVSTSGYSYLH

SEQ ID NO: 25 - protein, 7 aa, synthetic construct
LASYLES

SEQ ID NO: 26 - protein, 9 aa, synthetic construct
QHSRDLPLT

SEQ ID NO: 27 - protein, 120 aa, synthetic construct [sequence omitted]

SEQ ID NO: 28 - protein, 111 aa, synthetic construct [sequence omitted]

SEQ ID NO: 29 - protein, 447 aa, synthetic construct
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 30 - protein, 218 aa, synthetic construct
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 31 - protein, 5 aa, synthetic construct
NSGMH

SEQ ID NO: 32 - protein, 17 aa, synthetic construct
VIWYDGSKRYYADSVKG

SEQ ID NO: 33 - protein, 4 aa, synthetic construct
NDDY

SEQ ID NO: 34 - protein, 11 aa, synthetic construct
RASQSVSSYLA

SEQ ID NO: 35 - protein, 7 aa, synthetic construct
DASNRAT

SEQ ID NO: 36 - protein, 9 aa, synthetic construct
QQSSNWPRT

SEQ ID NO: 37 - protein, 113 aa, synthetic construct [sequence omitted]

SEQ ID NO: 38 - protein, 107 aa, synthetic construct [sequence omitted]

SEQ ID NO: 39 - protein, 440 aa, synthetic construct
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 40 - protein, 214 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 41 - protein, 5 aa, synthetic construct
NSGMH

SEQ ID NO: 42 - protein, 17 aa, synthetic construct
VIWYDGSKRYYADSVKG

SEQ ID NO: 43 - protein, 4 aa, synthetic construct
NDDY

SEQ ID NO: 44 - protein, 11 aa, synthetic construct
RASQSVSSYLA

SEQ ID NO: 45 - protein, 7 aa, synthetic construct
DASNRAT

SEQ ID NO: 46 - protein, 9 aa, synthetic construct
QQSSNWPRT

SEQ ID NO: 47 - protein, 117 aa, synthetic construct
EVQLVQSGAEVKKPGESLRISCKGSGYTFTTYWMHWVRQATGQGLEWMGNIYPGTGGSNFDEKFKNRVTITADKSTSTAYMELSSLRSEDTAVYYCTRWTTGTGAYWGQGTTVTVSS

SEQ ID NO: 48 - protein, 113 aa, synthetic construct
EIVLTQSPATLSLSPGERATLSCKSSQSLLDSGNQKNFLTWYQQKPGQAPRLLIYWASTRESGVPSRFSGSGSGTDFTFTISSLEAEDAATYYCQNDYSYPYTFGQGTKVEIK

SEQ ID NO: 49 - protein, 443 aa, synthetic construct
EVQLVQSGAEVKKPGESLRISCKGSGYTFTTYWMHWVRQATGQGLEWMGNIYPGTGGSNFDEKFKNRVTITADKSTSTAYMELSSLRSEDTAVYYCTRWTTGTGAYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 50 - protein, 220 aa, synthetic construct
EIVLTQSPATLSLSPGERATLSCKSSQSLLDSGNQKNFLTWYQQKPGQAPRLLIYWASTRESGVPSRFSGSGSGTDFTFTISSLEAEDAATYYCQNDYSYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 51 - protein, 5 aa, synthetic construct
DSWIH

SEQ ID NO: 52 - protein, 10 aa, synthetic construct
WISPYGGSTY

SEQ ID NO: 53 - protein, 7 aa, synthetic construct
RHWPGGF

SEQ ID NO: 54 - protein, 7 aa, synthetic construct
DVSTAVA

SEQ ID NO: 55 - protein, 6 aa, synthetic construct
SASFLY

SEQ ID NO: 56 - protein, 9 aa, synthetic construct
QQYLYHPAT

SEQ ID NO: 57 - protein, 115 aa, synthetic construct
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVT

SEQ ID NO: 58 - protein, 107 aa, synthetic construct [sequence omitted]

SEQ ID NO: 59 - protein, 448 aa, synthetic construct
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 60 - protein, 214 aa, synthetic construct
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 61 - protein, 5 aa, synthetic construct
SYIMM

SEQ ID NO: 62 - protein, 10 aa, synthetic construct
SIYPSGGITF

SEQ ID NO: 63 - protein, 9 aa, synthetic construct
IKLGTVTTV

SEQ ID NO: 64 - protein, 8 aa, synthetic construct
VGGYNYVS

SEQ ID NO: 65 - protein, 6 aa, synthetic construct
DVSNRP

SEQ ID NO: 66 - protein, 10 aa, synthetic construct
SSYTSSSTRV

SEQ ID NO: 67 - protein, 117 aa, synthetic construct
EVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVT

SEQ ID NO: 68 - protein, 110 aa, synthetic construct
QSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRVFGTGTKVTVL

SEQ ID NO: 69 - protein, 450 aa, synthetic construct
EVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 70 - protein, 216 aa, synthetic construct
QSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRVFGTGTKVTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 71 - protein, 5 aa, synthetic construct
RYWMS

SEQ ID NO: 72 - protein, 10 aa, synthetic construct
NIKQDGSEKY

SEQ ID NO: 73 - protein, 10 aa, synthetic construct
EGGWFGELAF

SEQ ID NO: 74 - protein, 8 aa, synthetic construct
RVSSSYLA

SEQ ID NO: 75 - protein, 6 aa, synthetic construct
DASSRA

SEQ ID NO: 76 - protein, 9 aa, synthetic construct
QQYGSLPWT

SEQ ID NO: 77 - protein, 118 aa, synthetic construct
EVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVT

SEQ ID NO: 78 - protein, 108 aa, synthetic construct
EIVLTQSPGTLSLSPGERATLSCRASQRVSSSYLAWYQQKPGQAPRLLIYDASSRATGIPDRFSGSGSGTDFTLTISRLEPEDFAVYYCQQYGSLPWTFGQGTKVEIK

SEQ ID NO: 79 - protein, 451 aa, synthetic construct
EVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 80 - protein, 215 aa, synthetic construct
EIVLTQSPGTLSLSPGERATLSCRASQRVSSSYLAWYQQKPGQAPRLLIYDASSRATGIPDRFSGSGSGTDFTLTISRLEPEDFAVYYCQQYGSLPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 81 - protein, 288 aa, Homo sapiens
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 82 - protein, 290 aa, Homo sapiens
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 83 - protein, 223 aa, Homo sapiens
MACLGFQRHKAQLNLATRTWPCTLLFFLLFIPVFCKAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDFLLWILAAVSSGLFFYSFLLTAVSLSKMLKKRSPLTTGVYVKMPPTEPECEKQFQPYFIPIN

SEQ ID NO: 84 - protein, 8 aa, synthetic construct
GFTFSSYT

SEQ ID NO: 85 - protein, 10 aa, synthetic construct
TFISYDGNNK

SEQ ID NO: 86 - protein, 11 aa, synthetic construct
ARTGWLGPFDY

SEQ ID NO: 87 - protein, 7 aa, synthetic construct
QSVGSSY

SEQ ID NO: 88 - skipped

SEQ ID NO: 89 - protein, 9 aa, synthetic construct
QQYGSSPWT

SEQ ID NO: 90 - protein, 118 aa, synthetic construct
QVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSS

SEQ ID NO: 91 - protein, 109 aa, synthetic construct
EIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIKR

SEQ ID NO: 92 - protein, 448 aa, synthetic construct
QVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 93 - protein, 215 aa, synthetic construct
EIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 94 - protein, 8 aa, synthetic construct
GFTFSSYG

SEQ ID NO: 95 - protein, 10 aa, synthetic construct
AVIWYDGSNK

SEQ ID NO: 96 - protein, 18 aa, synthetic construct
ARDPRGATLYYYYYGMDV

SEQ ID NO: 97 - protein, 6 aa, synthetic construct
QSINSY

SEQ ID NO: 98 - skipped

SEQ ID NO: 99 - protein, 9 aa, synthetic construct
QQYYSTPFT

SEQ ID NO: 100 - protein, 125 aa, synthetic construct
QVQLVESGGGVVQPGRSLRLSCAASGFTFSSYGMHWVRQAPGKGLEWVAVIWYDGSNKYYADSVKGRFTISRDNSKNTLYLQMNSLRAEDTAVYYCARDPRGATLYYYYYGMDVWGQGTTVTVSS

SEQ ID NO: 101 - protein, 108 aa, synthetic construct
DIQMTQSPSSLSASVGDRVTITCRASQSINSYLDWYQQKPGKAPKLLIYAASSLQSGVPSRFSGSGSGTDFTLTISSLQPEDFATYYCQQYYSTPFTFGPGTKVEIKR

SEQ ID NO: 102 - protein, 451 aa, synthetic construct
QVQLVESGGGVVQPGRSLRLSCAASGFTFSSYGMHWVRQAPGKGLEWVAVIWYDGSNKYYADSVKGRFTISRDNSKNTLYLQMNSLRAEDTAVYYCARDPRGATLYYYYYGMDVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 103 - protein, 214 aa, synthetic construct
DIQMTQSPSSLSASVGDRVTITCRASQSINSYLDWYQQKPGKAPKLLIYAASSLQSGVPSRFSGSGSGTDFTLTISSLQPEDFATYYCQQYYSTPFTFGPGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 104 - protein, 241 aa, Homo sapiens
MAQHGAMGAFRALCGLALLCALSLGQRPTGGPGCGPGRLLLGTGTDARCCRVHTTRCCRDYPGEECCSEWDCMCVQPEFHCGDPCCTTCRHHPCPPGQGVQSQGKFSFGFQCIDCASGTFSGGHEGHCKPWTDCTQFGFLTVFPGNKTHNAVCVPGSPPAEPLGWLTVVLLAVAACVLLLTSAQLGLHIWQLRSQCMWPRETQLLLEVPPSTEDARSCQFPEEERGERSAEEKGRLGDLWV

SEQ ID NO: 105 - protein, 255 aa, Homo sapiens
MAQHGAMGAFRALCGLALLCALSLGQRPTGGPGCGPGRLLLGTGTDARCCRVHTTRCCRDYPGEECCSEWDCMCVQPEFHCGDPCCTTCRHHPCPPGQGVQSQGKFSFGFQCIDCASGTFSGGHEGHCKPWTDCCWRCRRRPKTPEAASSPRKSGASDRQRRRGGWETCGCEPGRPPGPPTAASPSPGAPQAAGALRSALGRALLPWQQKWVQEGGSDQRPGPCSSAAAAGPCRRERETQSWPPSSLAGPDGVGS

SEQ ID NO: 106 - protein, 234 aa, Homo sapiens
MAQHGAMGAFRALCGLALLCALSLGQRPTGGPGCGPGRLLLGTGTDARCCRVHTTRCCRDYPGEECCSEWDCMCVQPEFHCGDPCCTTCRHHPCPPGQGVQSQGKFSFGFQCIDCASGTFSGGHEGHCKPWTDCTQFGFLTVFPGNKTHNAVCVPGSPPAEPLGWLTVVLLAVAACVLLLTSAQLGLHIWQLRKTQLLLEVPPSTEDARSCQFPEEERGERSAEEKGRLGDLWV

SEQ ID NO: 107 - protein, 5 aa, synthetic construct
TYWMH

SEQ ID NO: 108 - protein, 17 aa, synthetic construct
NIYPGTGGSNFDEKFKN

SEQ ID NO: 109 - protein, 8 aa, synthetic construct
WTTGTGAY

SEQ ID NO: 110 - protein, 17 aa, synthetic construct
KSSQSLLDSGNQKNFLT